(19) United States
(12) Patent Application Publication
(10) Pub. No.: US 2024/0166622 A1
(43) Pub. Date: May 23, 2024

(54) FLUORINATED QUINOLINE, QUINOXALINE AND BENZO[B][1,4]OXAZINE DERIVATIVES AS DIHYDROOROTATE DEHYDROGENASE (DHODH) INHIBITORS FOR THE TREATMENT OF CANCER, AUTOIMMUNE AND INFLAMMATORY DISEASES

(71) Applicant: Janssen Biotech, Inc., Horsham, PA (US)

(72) Inventors: Justin Cisar, North Wales, PA (US); Scott Kuduk, Harleysville, PA (US); Aihua Wang, Jamison, PA (US); Zhuming Zhang, Hillsborough, NJ (US); Chao-yuan Wang, Elkins Park, PA (US)

(21) Appl. No.: 17/773,079

(22) PCT Filed: Oct. 30, 2020
(86) PCT No.: PCT/IB2020/060217

§ 371 Date: Apr. 29, 2022

Related U.S. Application Data
(60) Provisional application No. 62/929,160, filed on Nov. 1, 2019.

(51) Int. Cl. C07D 401/04; C07D 403/04; C07D 413/04

(52) U.S. Cl. CPC C07D 401/04; C07D 403/04; C07D 413/04

(57) ABSTRACT

Disclosed are compounds, compositions, and methods for treating diseases, disorders, or medical conditions that are affected by the modulation of DHODH. Such compounds are represented by Formula (I) as follows:

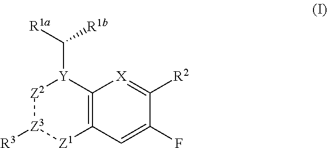

wherein X, Y, R^{1a}, R^{1b}, Z¹, Z², Z³, R², and R³, are defined herein.

### CROSS-REFERENCE TO RELATED APPLICATIONS

This application claims the benefit of priority of U.S. Provisional Application No. 62/929,160, filed on Nov. 1, 2019, which is incorporated by reference herein, in its entirety and for all purposes.

### FIELD OF THE INVENTION

The present invention relates to novel compounds that are dihydroorotate dehydrogenase (DHODH) inhibitors. These compounds may be useful for the treatment of a disease, disorder, or medical condition where there is an advantage in inhibiting DHODH. The invention also relates to pharmaceutical compositions comprising one or more of such compounds, to processes to prepare such compounds and compositions, and to the use of such compounds or pharmaceutical compositions for the method of treatment of cancer, and autoimmune and inflammatory diseases, syndromes, and disorders.

### BACKGROUND OF THE INVENTION

Acute myelogenous leukemia (AML) is a clonal disease of the blood and bone marrow resulting from mutations that occur in normal hematopoietic stem cells. AML is a heterogenous disease in that it presents with a range of cytogenetic, morphological and immunophenotypic features, and is characterized by an accumulation of clonal, abnormal myeloid progenitor cells, known as myeloblasts. These cells demonstrate disruption of normal myeloid differentiation and excessive proliferation, resulting in the decreased formation of hematopoietic cells. Disease remission can be achieved with standard induction chemotherapy, but refractory and relapsed disease remains a challenge due to persistence of leukemic stem cells. Therefore, AML represents an unmet medical need with >20,000 new cases per year in the US with 5-year overall survival below 30% (Stein E T et al., Health Qual Life Outcomes 16: 193, 2018).

Differentiation therapy is considered an attractive approach to AML treatment based on the knowledge that differentiation and loss of stem cell self-renewal are coupled in normal cells. Treatment of acute promyelocytic leukemia, which represents 10-15% of all AML, with all-trans retinoic acid is the paradigm for differentiation therapy. Retinoic acid targets the promyelocytic leukemia protein (PML)-retinoic acid receptor-α (RAR-α) fusion protein encoded by a t(15,17) chromosomal translocation. Targeting PML-RAR specifically lifts the transcriptionally mediated differentiation block induced by the fusion protein and early clinical trials with single agent ATRA demonstrated complete hematologic remission in all treated patients (McCulloch D et al. Onco Targets Ther 2017; 10: 1585-1601; Nowak D et al. Blood 113: 3655, 2009).

Although differentiation therapy is successful, it is only applicable to a small population of AML patients. Research efforts have aimed at identifying additional differentiation inducing agents, but with limited success. Recently dihydroorotate dehydrogenase (DHODH) emerged as a potentially more broadly applicable differentiation target in a phenotypic screen aimed at identifying small molecules that overcome blockade of the maturation of primary murine bone marrow cells expressing the homeobox protein HoxA9. This protein is a key transcription factor involved in balancing stem cell maintenance/differentiation and is normally expressed in hematopoietic progenitor cells and downregulated upon induction of differentiation and has been found to be widely overexpressed in AML (Sykes et al., Cell 167: 171, 2016).

DHODH is a flavin mononucleotide (FMN) flavoprotein located in the inner mitochondrial membrane that catalyzes the oxidation of dihydroorotate to orotate, the fourth step in the de novo pyrimidine biosynthesis pathway. Inhibition of DHODH leads to decreased pyrimidine synthesis important precursors for nucleotide synthesis, but also glycoprotein and phospholipid biosynthesis (Reis RAG et al., Archives Biochem Biophysics 632: 175, 2017; Vyas V K et al., Mini Rev Med Chem 11: 1039, 2011). DHODH is a validated target for the treatment of autoimmune diseases with the FDA approved small molecule DHODH inhibitors leflunomide and teriflunomide for rheumatoid arthritis and multiple sclerosis, respectively (Lolli M L et al., Recent patents on Anti-Cancer Drug Discovery 13: 86, 2018).

Since the first observation by Sykes et al. demonstrating that DHODH inhibition drives AML differentiation in vitro, as evidenced by upregulation of the differentiation markers CD11b and CD14, and results in dose dependent anti-leukemic effects, decreased leukemic stem cells and prolonged survival in vivo, additional evidence emerged demonstrating that small molecule DHODH inhibitors mediate antiproliferative activity against AML cells with concomitant cell cycle arrest, upregulation of CD11b and CD14, and induction of apoptosis (Wu D et al. Haematologica 103: 1472, 2018; Sainas S et al., J Med Chem 61: 6034, 2018; Cao L et al., Mol Cancer Ther, October 23rd Epub ahead of print). Moreover, preclinical solid tumor in vitro and in vivo models demonstrated effectiveness of DHODH inhibition and DHODH was identified as a synthetic lethality in PTEN and KRAS mutant solid tumors (Pharmacology and Therapeutics, Epub Oct. 19, 2018; Mathur D et al., Cancer Discovery 7: 1, 2017; Cell Chemical Biology 25: 1, 2018).

Thus, there remains a need for DHODH inhibitors that provide a therapeutic benefit to patients suffering from cancer and/or inflammatory and immunological diseases.

### SUMMARY OF THE INVENTION

Embodiments of the present invention relate to compounds, pharmaceutical compositions containing them, methods of making and purifying them, methods of using them as inhibitors of DHODH enzymatic activity and methods for using them in the treatment of a subject suffering from or diagnosed with a disease, disorder, or medical condition such as autoimmune or inflammatory disorders, or diseases such as cancer.

Embodiments of this invention are compounds of Formula (I),

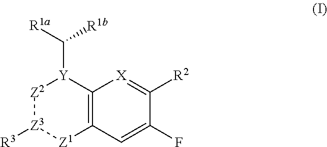

wherein
X is CH or N;
Y is CH or N;
Z¹ is selected from the group consisting of: CH₂, C(CH₃), CH(OH), C(CH₃)(OH), O, C═O, and NR^{a};
   R^{a} is selected from the group consisting of: H, CH₂(C═O)NH₂, (C═O)CH₃, and (C═O)NHCH₃;
Z² is CH, CH₂, or C═O;
Z³ is C, CH or C(CH₃);
each [symbol] is independently a single bond or a double bond;
   wherein
   when Z³ is CH or C(CH₃), [symbol] between Z² and Z³ is a single bond, and [symbol] between Z³ and Z¹ is a single bond;
   when Z³ is C, Z² is CH, [symbol] between Z² and Z³ is a double bond, and [symbol] between Z³ and Z¹ is a single bond;
   or
   when Z¹ is C(CH₃), [symbol] between Z² and Z³ is a single bond, and [symbol] between Z³ and Z¹ is a double bond;
R^{1a} is selected from the group consisting of: C₁₋₆alkyl; C₁₋₆alkyl substituted with OH, or OCH₃; C₁₋₆haloalkyl; C₁₋₆haloalkyl substituted with OH, or OCH₃; and C₃₋₆cycloalkyl;
R^{1b} is CH₃ or CHF₂; or R^{1a} and R^{1b} come together to form C₃₋₆cycloalkyl; C₃₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl; R² is

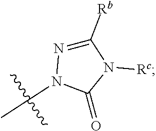

wherein
   R^{b} is C₁₋₆alkyl substituted with a member selected from the group consisting of OH, halo, CN, OC₁₋₆alkyl, OC₁₋₆haloalkyl and OC₃₋₆cycloalkyl; and
   R^{c} is selected from the group consisting of C₁₋₆alkyl, C₁₋₆haloalkyl, and C₃₋₆cycloalkyl;
R³ is

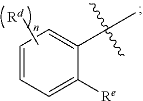

wherein
   R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
   R^{e} is selected from the group consisting of H, halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of OH, and OCH₃; OH; OC₁₋₆alkyl; and C₃₋₆cycloalkyl;
   n is 1, or 2; and
   R⁴ is H or CH₃;
      or a pharmaceutically acceptable salt, isotope, N-oxide, solvate, or stereoisomer thereof.

The present invention further provides methods for treating or ameliorating a disease, syndrome, condition, or disorder in a subject, including a mammal and/or human in which the disease, syndrome, condition, or disorder is affected by the inhibition of DHODH enzymatic activity, including but not limited to, cancer and/or inflammatory or immunological diseases, using a compound of Formula (I) or a pharmaceutically acceptable salt, isotope, N-oxide, solvate, or stereoisomer thereof.

Additional embodiments, features, and advantages of the invention will be apparent from the following detailed description and through practice of the invention.

### DETAILED DESCRIPTION OF THE INVENTION

Unless defined otherwise, all technical and scientific terms used herein have the same meaning as is commonly understood by one of ordinary skill in art. As used in the specification and the appended claims, unless specified to the contrary, the following terms have the meaning indicated in order to facilitate the understanding of the present invention.

The singular forms “a”, “an” and “the” encompass plural references unless the context clearly indicates otherwise.

With reference to substituents, the term “independently” refers to the situation where when more than one substituent is possible, the substituents may be the same or different from each other.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

Unless qualified specifically in particular instances of use, the term “alkyl” refers to a straight- or branched-chain alkyl group having from 1 to 8 carbon atoms in the chain. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. “C₁₋₆alkyl” refers to straight- or branched-chain alkyl group having from 1 to 6 carbon atoms in the chain. “C₁₋₄alkyl” refers to straight- or branched-chain alkyl group having from 1 to 4 carbon atoms in the chain.

The term “cycloalkyl” refers to a saturated or partially saturated, monocyclic, fused polycyclic, or spiro polycyclic carbocycle having from 3 to 12 ring atoms per carbocycle. “C₃₋₆cycloalkyl” refers to a carbocycle having from 3 to 6 ring atoms per carbocycle. Illustrative examples of cycloalkyl groups include the following entities, in the form of properly bonded moieties:

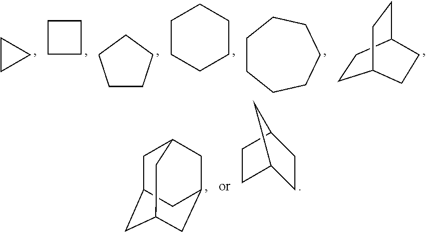

The term “halogen” or “halo” represents chlorine, fluorine, bromine, or iodine.

The term “haloalkyl” refers to a straight- or branched-chain alkyl group having from 1 to 6 carbon atoms in the chain optionally substituting hydrogens with halogens. The term “C₁₋₆haloalkyl” as used here refers to a straight- or branched-chain alkyl group having from 1 to 6 carbon atoms in the chain, optionally substituting hydrogens with halogens. The term “C₁₋₄ haloalkyl” as used here refers to a straight- or branched-chain alkyl group having from 1 to 4 carbon atoms in the chain, optionally substituting hydrogens with halogens. Examples of “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (CF(CF₃)₂), and groups that in light of the ordinary skill in the art and the teachings provided herein would be considered equivalent to any one of the foregoing examples.

The term “aryl” refers to a monocyclic, aromatic carbocycle (ring structure having ring atoms that are all carbon) having 6 atoms per ring. (Carbon atoms in the aryl groups are sp2 hybridized.) The term “phenyl” represents the following moiety:

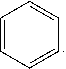

The term “oxetanyl” represents the following moiety:

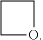

The term “tetrahydrofuranyl” represents the following moiety:

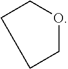

The term “tetrahydropyranyl” represents the following moiety:

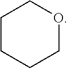

The term “heteroaryl” refers to a monocyclic or fused bicyclic heterocycle (ring structure having ring atoms selected from carbon atoms and up to four heteroatoms selected from nitrogen, oxygen, and sulfur) having from 3 to 9 ring atoms per heterocycle. Illustrative examples of heteroaryl groups include the following entities, in the form of properly bonded moieties:

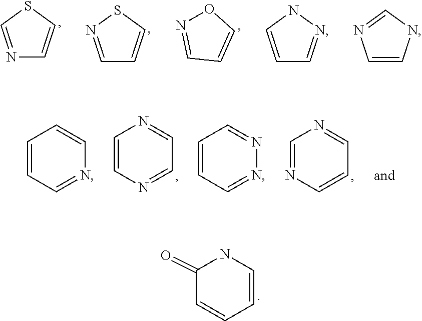

The term “tautomeric” or “tautomeric form” refers to structural isomers of different energies that are interconvertible through low energy barriers. For example, proton tautomers (also known as proton tautomers) include interconversions through the transfer of protons, such as keto-enol and imine-enamine isomerization. The valence tautomers include interconversions by restructuring some bond electrons.

For example, hydroxypyridine or the tautomeric pyridone is represented below.

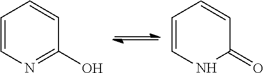 [SMILES: Oc1ccccn1 ; O=c1cccc[nH]1]

For example, pyrazole tautomers are represented below.

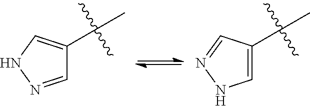 [SMILES: *c1cn[nH]c1 ; *c1cn[nH]c1]

Those skilled in the art will recognize that the species of heterocycloalkyl, cycloalkyl, heteroaryl and aryl groups listed or illustrated above are not exhaustive, and that additional species within the scope of these defined terms may also be selected.

The term “variable point of attachment” means that a group is allowed to be attached at more than one alternative position in a structure. The attachment will always replace a hydrogen atom on one of the ring atoms. In other words, all permutations of bonding are represented by the single diagram, as shown in the illustrations below.

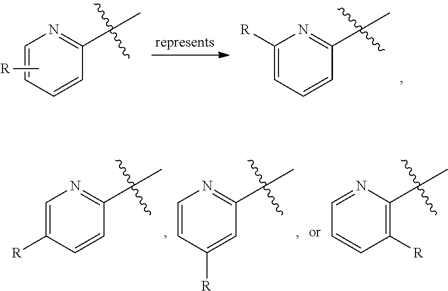

Those skilled in the art will recognize that that if more than one such substituent is present for a given ring; the bonding of each substituent is independent of all of the others. The groups listed or illustrated above are not exhaustive.

As used herein, the term “or” means “and/or” unless stated otherwise.

As used herein, the terms “including”, “containing” and “comprising” are used in their open, non-limiting sense.

As used herein, the term “composition” is intended to encompass a product comprising the specified ingredients in the specified amounts, as well as any product which results, directly or indirectly, from combination of the specified ingredients in the specified amounts.

As used herein, the term “treat”, “treating”, or “treatment” of any disease, condition, syndrome or disorder refers, in one embodiment, to ameliorating the disease, condition, syndrome or disorder (i.e. slowing or arresting or reducing the development of the disease or at least one of the clinical symptoms thereof). In another embodiment, “treat”, “treating”, or “treatment” refers to alleviating or ameliorating at least one physiological or biochemical parameter associated with or causative of the disease, condition, syndrome, or disorder, including those which may not be discernible by the patient. In a further embodiment, “treat”, “treating”, or “treatment” refers to modulating the disease, condition, syndrome, or disorder either physically (e.g. stabilization of a discernible symptom), physiologically, (e.g. stabilization of a physical parameter), or both. In yet another embodiment, “treat”, “treating”, or “treatment” refers to preventing or delaying the onset or development or progression of the disease, condition, syndrome or disorder.

The terms “subject” and “patient” are used interchangeably herein and may refer to an animal, preferably a mammal, most preferably a human.

As used herein, the terms active compound, pharmaceutical agent and active ingredient are used interchangeably to refer to a pharmaceutically active compound. Other ingredients in a drug composition, such as carriers, diluents or excipients, may be substantially or completely pharmaceutically inert. A pharmaceutical composition (also referred to herein as a composition or formulation) may comprise the active ingredient in combination with one or more carriers and/or one or more excipients and/or one or more diluents.

The term “therapeutically effective amount” (used interchangeably herein with “effective amount”) refers to an amount (e.g., of an active compound or pharmaceutical agent, such as a compound of the present invention), which elicits the biological or medicinal response in a tissue system, animal or human that is being sought by a researcher, veterinarian, medical doctor or other clinician, including reduction or inhibition of an enzyme or a protein activity, or ameliorating symptoms, alleviating conditions, slowing or delaying disease progression, or preventing a disease. Stated another way, the term therapeutically effective amount may refer to an amount that, when administered to a particular subject, achieves a therapeutic effect by inhibiting, alleviating or curing a disease, condition, syndrome or disorder in the subject or by prophylactically inhibiting, preventing or delaying the onset of a disease, condition, syndrome or disorder, or symptom(s) thereof. A therapeutically effective amount may be an amount which relieves to some extent one or more symptoms of a disease, condition, syndrome or disorder in a subject; and/or returns to normal either partially or completely one or more physiological or biochemical parameters associated with or causative of the disease, condition, syndrome or disorder; and/or reduces the likelihood of the onset of the disease, condition, syndrome or disorder, or symptom(s) thereof.

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

A “pharmaceutically acceptable salt” is intended to mean a salt of an acid or base of a compound represented by Formula (I) (as well as compounds of Formula (IA), (IB), (IC), (ID), and (IE)) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response.

Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt.

Compounds of Formula (I) may contain at least one nitrogen of basic character, so desired pharmaceutically acceptable salts may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents.

Compounds of Formula (I) may contain a carboxylic acid moiety, a desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, piperazine, N-methyl-glucamine and tromethamine and inorganic salts derived from sodium, calcium, potassium, magnesium, manganese, iron, copper, zinc, aluminum, and lithium.

Each compound used herein may be discussed interchangeably with respect to its chemical formula, chemical name, abbreviation, etc.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of such formula. The compounds of this invention may possess one or more asymmetric centers; such compounds can therefore be produced as individual (R)- or (S)-stereoisomers or as mixtures thereof. Thus, any formula given herein is intended to represent a racemate, one or more of its enantiomeric forms, one or more of its diastereomeric forms, and mixtures thereof. Additionally, any formula given herein is intended to refer also to any one of: hydrates, solvates, polymorphs and of such compounds, and mixtures thereof, even if such forms are not listed explicitly.

The term “R” at a stereocenter designates that the stereocenter is purely of the R-configuration as defined in the art; likewise, the term “S” means that the stereocenter is purely of the S-configuration. As used herein, the term “RS” refers to a stereocenter that exists as a mixture of the R- and S-configurations.

Compounds containing one stereocenter drawn without a stereo bond designation are a mixture of 2 enantiomers. Compounds containing 2 stereocenters both drawn without stereo bond designations are a mixture of 4 diastereomers. Compounds with 2 stereocenters both labeled “RS” and drawn with stereo bond designations are a 2-component mixture with relative stereochemistry as drawn. Unlabeled stereocenters drawn without stereo bond designations are a mixture of the R- and S-configurations. For unlabeled stereocenters drawn with stereo bond designations, the absolute stereochemistry is as depicted.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

Reference to a compound herein stands for a reference to any one of: (a) the recited form of such compound, and (b) any of the forms of such compound in the medium in which the compound is being considered when named. For example, reference herein to a compound such as R—COOH, encompasses reference to any one of: for example, R—COOH(s), R—COOH(sol), and R—COO-(sol). In this example, R—COOH(s) refers to the solid compound, as it could be for example in a tablet or some other solid pharmaceutical composition or preparation; R—COOH(sol) refers to the undissociated form of the compound in a solvent; and R—COO-(sol) refers to the dissociated form of the compound in a solvent, such as the dissociated form of the compound in an aqueous environment, whether such dissociated form derives from R—COOH, from a salt thereof, or from any other entity that yields R—COO— upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH(aq) and/or R—COO-(aq), where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number in an enriched form. Examples of isotopes that can be incorporated into compounds of the invention in a form that exceeds natural abundances include isotopes of hydrogen, carbon, nitrogen, oxygen, phosphorous, fluorine, chlorine, and iodine, such as ²H (or chemical symbol D), ³H (or chemical symbol T), ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H, or D) may afford certain therapeutic advantages resulting from greater metabolic stability, for example increased in vivo half-life or reduced dosage requirements. Isotopically labeled compounds of this invention can generally be prepared by carrying out the procedures disclosed in the schemes or in the examples and preparations described below by substituting a readily available isotopically labeled reagent for a non-isotopically labeled reagent.

The term Cₙ₋ₘ alkyl refers to an aliphatic chain, whether straight or branched, with a total number N of carbon members in the chain that satisfies n≤N≤m, with m>n.

When the same plurality of substituents is assigned to various groups, the specific individual substituent assignment to each of such groups is meant to be independently made with respect to the specific individual substituent assignments to the remaining groups. By way of illustration, but not as a limitation, if each of groups Q and R can be H or F, the choice of H or F for Q is made independently of the choice of H or F for R, so the choice of assignment for Q does not determine or condition the choice of assignment for R, or vice-versa, unless it is expressly indicated otherwise. Illustrative claim recitation in this regard would read as “each of Q and R is independently H or F”, or “each of Q and R is independently selected from the group consisting of H and F”.

In another example, a zwitterionic compound would be encompassed herein by referring to a compound that is known to form a zwitterion, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification CHEBI:27369 by the Chemical Entities of Biological Interest (ChEBI) dictionary of molecular entities. As generally well known, a zwitterion or zwitterionic compound is a neutral compound that has formal unit charges of opposite sign. Sometimes these compounds are referred to by the term “inner salts”. Other sources refer to these compounds as “dipolar ions”, although the latter term is regarded by still other sources as a misnomer. As a specific example, aminoethanoic acid (the amino acid glycine) has the formula H₂NCH₂COOH, and it exists in some media (in this case in neutral media) in the form of the zwitterion+H₃NCH₂COO⁻. Zwitterions, zwitterionic compounds, inner salts, and dipolar ions in the known and well-established meanings of these terms are within the scope of this invention, as would in any case be so appreciated by those of ordinary skill in the art. Because there is no need to name each and every embodiment that would be recognized by those of ordinary skill in the art, no structures of the zwitterionic compounds that are associated with the compounds of this invention are given explicitly herein. They are, however, part of the embodiments of this invention. No further examples in this regard are provided herein because the interactions and transformations in a given medium that lead to the various forms of a given compound are known by any one of ordinary skill in the art.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this invention given according to the choices S¹ₑₓₐₘₚₗₑ is S₁ and S²ₑₓₐₘₚₗₑ is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein.

Furthermore, when more than one assignment is given for any member or substituent, embodiments of this invention comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this invention for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein.

The nomenclature “Cᵢ-Cⱼ” with j>i, when applied herein to a class of substituents, is meant to refer to embodiments of this invention for which each and every one of the number of carbon members, from i to j including i and j, is independently realized. By way of example, the term C₁-C₃ refers independently to embodiments that have one carbon member (C₁), embodiments that have two carbon members (C₂), and embodiments that have three carbon members (C₃).

Embodiments of this invention include compounds of Formula (I),

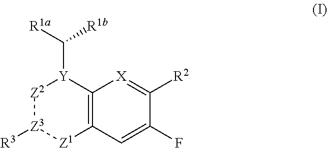

wherein
X is CH or N;
Y is CH or N;
Z¹ is selected from the group consisting of: CH₂, C(CH₃), CH(OH), C(CH₃)(OH), O, C═O, and NR^{a};
   R^{a} is selected from the group consisting of: H, CH₂(C═O)NH₂, (C═O)CH₃, and (C═O)NHCH₃;
Z² is CH, CH₂, or C═O;
Z³ is C, CH or C(CH₃);
each [symbol] is independently a single bond or a double bond;
   wherein
   when Z³ is CH or C(CH₃), [symbol] between Z² and Z³ is a single bond, and [symbol] between Z³ and Z¹ is a single bond;
   when Z³ is C, Z² is CH, [symbol] between Z² and Z³ is a double bond, and [symbol] between Z³ and Z¹ is a single bond;
   or
   when Z¹ is C(CH₃), [symbol] between Z² and Z³ is a single bond, and [symbol] between Z³ and Z¹ is a double bond;
R^{1a} is selected from the group consisting of: C₁₋₆alkyl; C₁₋₆alkyl substituted with OH, or OCH₃; C₁₋₆haloalkyl; C₁₋₆haloalkyl substituted with OH, or OCH₃; and C₃₋₆cycloalkyl;
R^{1b} is CH₃ or CHF₂; or R^{1a} and R^{1b} come together to form C₃₋₆cycloalkyl; C₃₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl;
R² is

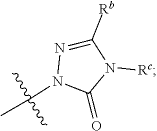

wherein
   R^{b} is C₁₋₆alkyl substituted with a member selected from the group consisting of: OH, halo, CN, OC₁₋₆alkyl, OC₁₋₆haloalkyl and OC₃₋₆cycloalkyl; and
   R^{c} is selected from the group consisting of: C₁₋₆alkyl, C₁₋₆haloalkyl, and C₃₋₆cycloalkyl;
R³ is

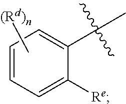

wherein
   R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
   R^{e} is selected from the group consisting of: H, halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl;
   n is 1, or 2; and
   R⁴ is H or CH₃;
      or pharmaceutically acceptable salts, isotopes, tautomers, N-oxides, solvates, or stereoisomers thereof.

Embodiments of this invention include compounds of Formula (I),

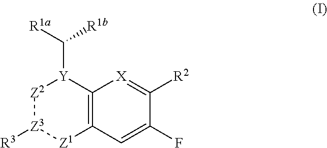

wherein
X is CH;
Y is CH or N;
Z¹ is selected from the group consisting of: CH₂, C(CH₃), CH(OH), C(CH₃)(OH), O, C═O, and NR^{a};
   R^{a} is selected from the group consisting of: H, CH₂(C═O)NH₂, (C═O)CH₃, and (C═O)NHCH₃;
Z² is CH, CH₂, or C═O;
Z³ is C, CH or C(CH₃);
each [symbol] is independently a single bond or a double bond;
   wherein
   when Z³ is CH or C(CH₃), [symbol] between Z² and Z³ is a single bond, and [symbol] between Z³ and Z¹ is a single bond;
   when Z³ is C, Z² is CH, [symbol] between Z² and Z³ is a double bond, and [symbol] between Z³ and Z¹ is a single bond;
   or
   when Z¹ is C(CH₃), [symbol] between Z² and Z³ is a single bond, and [symbol] between Z³ and Z¹ is a double bond;
R^{1a} is selected from the group consisting of: C₁₋₆alkyl; C₁₋₆alkyl substituted with OH, or OCH₃; C₁₋₆haloalkyl; C₁₋₆haloalkyl substituted with OH, or OCH₃; and C₃₋₆cycloalkyl;
R^{1b} is CH₃ or CHF₂; or R^{1a} and R^{1b} come together to form C₃₋₆cycloalkyl; C₃₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl;
R² is

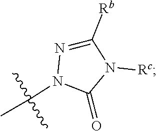

wherein
   R^{b} is C₁₋₆alkyl substituted with a member selected from the group consisting of: OH, halo, CN, OC₁₋₆alkyl, OC₁₋₆haloalkyl and OC₃₋₆cycloalkyl; and
   R^{c} is selected from the group consisting of: C₁₋₆alkyl, C₁₋₆haloalkyl, and C₃₋₆cycloalkyl;
R³ is

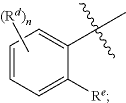

wherein
   R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
   R^{e} is selected from the group consisting of: H, halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl;
   n is 1, or 2; and
   R⁴ is H or CH₃;
      or pharmaceutically acceptable salts, isotopes, tautomers, N-oxides, solvates, or stereoisomers thereof.

An additional embodiment of the invention is a compound of Formula (I) wherein X is CH.

An additional embodiment of the invention is a compound of Formula (I) wherein Y is CH.

An additional embodiment of the invention is a compound of Formula (I) wherein Y is N.

An additional embodiment of the invention is a compound of Formula (I) wherein

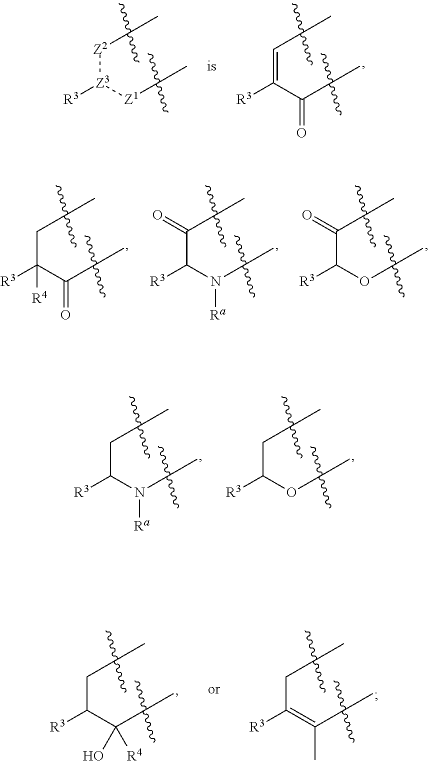

and R⁴ is H or CH₃.

An additional embodiment of the invention is a compound of Formula (I) wherein

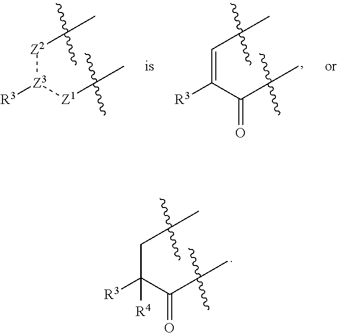

An additional embodiment of the invention is a compound of Formula (I) wherein

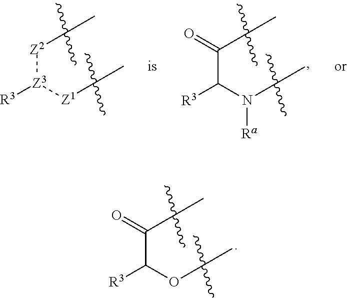

An additional embodiment of the invention is a compound of Formula (I) wherein

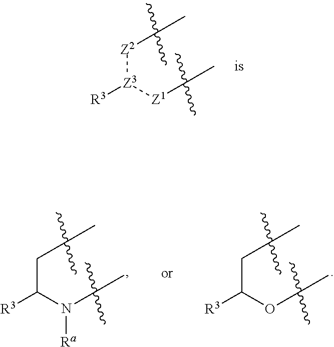

An additional embodiment of the invention is a compound of Formula (I) wherein

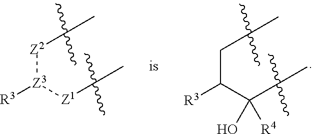

An additional embodiment of the invention is a compound of Formula (I) wherein

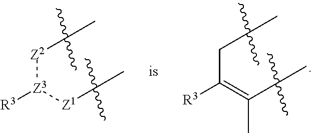

An additional embodiment of the invention is a compound of Formula (I) wherein R^{a} is C₁₋₄alkyl substituted with OH; CH₂(C═O)NH₂, (C═O)CH₃, and (C═O)NHCH₃.

An additional embodiment of the invention is a compound of Formula (I) wherein R^{1a} is C₁₋₄alkyl; C₁₋₄alkyl substituted with OH, or OCH₃; C₁₋₄haloalkyl; C₁₋₄haloalkyl substituted with OH, or OCH₃; or C₃₋₆cycloalkyl.

An additional embodiment of the invention is a compound of Formula (I) wherein R^{1a} is CH₃ or CF₃.

An additional embodiment of the invention is a compound of Formula (I) wherein R^{1b} is CH₃ or CHF₂.

An additional embodiment of the invention is a compound of Formula (I) wherein R^{1b} is CH₃.

An additional embodiment of the invention is a compound of Formula (I) wherein R^{1a} and R^{1b} come together to form cyclopropyl, cyclobutyl, cyclopentyl, or cyclohexyl; cyclopropyl, cyclobutyl, cyclopentyl, or cyclohexyl each independently substituted with one, two, three or four members selected from the group consisting of halo, OH, C₁₋₄alkyl, and C₁₋₄haloalkyl; oxetanyl; tetrahydrofuranyl; and tetrahydropyranyl.

An additional embodiment of the invention is a compound of Formula (I) wherein R² is

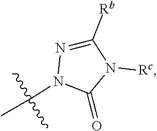

where
R^{b} is C₁₋₄alkyl substituted with OH, halo, CN, OC₁₋₄alkyl, OC₁₋₄haloalkyl or OC₃₋₆cycloalkyl; and
   R^{c} is C₁₋₄alkyl, C₁₋₄haloalkyl, or C₃₋₆cycloalkyl.

An additional embodiment of the invention is a compound of Formula (I) wherein R² is

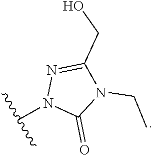 [SMILES: *n1nc(CO)n(CC)c1=O]

An additional embodiment of the invention is a compound of Formula (I) wherein R³ is

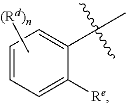

where
R^{d} is H; halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; CN; or OC₁₋₄alkyl;
R^{e} is H, halo; C₁₋₄alkyl; C₁₋₄alkyl substituted with OH, OCH₃, SCH₃, or OCF₃; C₁₋₄haloalkyl; or C₁₋₄haloalkyl substituted with OH, or OCH₃; and
n is 1 or 2.

An additional embodiment of the invention is a compound of Formula (I) wherein R^{e} is H, SCH₃, Cl, F, or CH₃.

An additional embodiment of the current invention is a compound selected from the compounds shown below in Table 1, and pharmaceutically acceptable salts, isotopes, N-oxides, solvates, and stereoisomers thereof.

TABLE 1

| Example # | Compound Name |
|---|---|
| 1 | 7-(4-Ethyl-3-(hydroxymethyl)-5-oxo-4,5-dihydro-1H-1,2,4-triazol-1-y1)-6- |
| | fluoro-3-(3-fluorophenyl)-1-isopropylquinolin-4(1H)-one; |
| 2 | 7-(4-Ethyl-3-(hydroxymethyl)-5-oxo-4,5-dihydro-1H-1,2,4-triazol-1-y1)-3-(2- |
| | (methylthio)phenyl)-1-((S)-1,1,1-trifluoropropan-2-yl)-3,4-dihydroquinoxalin- |
| | 2(1H)-one; |
| 3 | 3-(2-Chloro-6-fluorophenyl)-7-(4-ethyl-3-(hydroxymethyl)-5-oxo-4,5-dihydro- |
| | 1H-1,2,4-triazol-1-yl)-1-((S)-1,1,1-trifluoropropan-2-yl)-3,4-dihydroquinoxalin- |
| | 2(1H)-one; |
| 4 | 2-(2-(2-Chloro-6-fluorophenyl)-4-((S)-1,1,1-trifluoropropan-2-y1)-1,2,3,4- |
| | tetrahydroquinoxalin-6-yl)-4-ethyl-5-(hydroxymethyl)-2,4-dihydro-3H-1,2,4- |
| | triazol-3-one; |
| 5 | 2-(2-Chloro-6-fluorophenyl)-6-(4-ethyl-3-(hydroxymethyl)-5-oxo-4,5-dihydro- |
| | 1H-1,2,4-triazol-1-yl)-7-fluoro-4-isopropyl-2H-benzo[b][1,4]oxazin-3(4H)-one; |
| 6 | 1-(2-(2-Chloro-6-fluorophenyl)-7-fluoro-4-isopropyl-3,4-dihydro-2H- |
| | benzo[b][1,4]oxazin-6-yl)-4-ethyl-3-(hydroxymethyl)-1H-1,2,4-triazol-5(4H)- |
| | one; |
| 7 | (S*)-1-(2-(2-Chloro-6-fluorophenyl)-7-fluoro-4-isopropyl-3,4-dihydro-2H- |
| | benzo[b][1,4]oxazin-6-yl)-4-ethyl-3-(hydroxymethyl)-1H-1,2,4-triazol-5(4H)- |
| | one; |
| 8 | (R*)-1-(2-(2-Chloro-6-fluorophenyl)-7-fluoro-4-isopropyl-3,4-dihydro-2H- |
| | benzo[b][1,4]oxazin-6-y1)-4-ethyl-3-(hydroxymethyl)-1H-1,2,4-triazol-5(4H)- |
| | one; |
| 9 | 7-(4-Ethyl-3-(hydroxymethyl)-5-oxo-4,5-dihydro-1H-1,2,4-triazol-1-y1)-6- |
| | fluoro-3-(2-fluoro-5-methylpheny1)-1-isopropylquinolin-4(1H)-one; |
| 10 | 7-(4-Ethyl-3-(hydroxymethyl)-5-oxo-4,5-dihydro-1H-1,2,4-triazol-1-y1)-6- |
| | fluoro-1-isopropyl-3-(o-tolyl)quinolin-4(1H)-one; |
| 11 | 7-(4-Ethyl-3-(hydroxymethyl)-5-oxo-4,5-dihydro-1H-1,2,4-triazol-1-yl)-6- |
| | fluoro-1-isopropyl-3-(o-tolyl)-2,3-dihydroquinolin-4(1H)-one; |
| 12 | 3-(2-Chloro-6-fluorophenyl)-7-(4-ethyl-3-(hydroxymethyl)-5-oxo-4,5-dihydro- |
| | 1H-1,2,4-triazol-1-y1)-6-fluoro-1-isopropylquinolin-4(1H)-one; |
| 13 | Racemic 4-Ethyl-2-(7-fluoro-4-isopropyl-2-(o-tolyl)-1,2,3,4-tetrahydroquinolin- |
| | 6-yl)-5-(hydroxymethyl)-2,4-dihydro-3H-1,2,4-triazol-3-one; |
| 14 | Racemic 4-Ethyl-2-(6-fluoro-4-hydroxy-1-isopropyl-3-(o-tolyl)-1,2,3,4- |
| | tetrahydroquinolin-7-y1)-5-(hydroxymethyl)-2,4-dihydro-3H-1,2,4-triazol-3-one; |
| 15 | 4-Ethyl-2-((3S*,4S*)-6-fluoro-4-hydroxy-1-isopropyl-3-(o-tolyl)-1,2,3,4- |
| | tetrahydroquinolin-7-yl)-5-(hydroxymethyl)-2,4-dihydro-3H-1,2,4-triazol-3-one; |
| 16 | 4-Ethyl-2-((3R*,4R*)-6-fluoro-4-hydroxy-1-isopropyl-3-(o-toly1)-1,2,3,4- |
| | tetrahydroquinolin-7-yl)-5-(hydroxymethyl)-2,4-dihydro-3H-1,2,4-triazol-3-one; |
| 17 | 4-Ethyl-2-((3S*,4R*)-6-fluoro-4-hydroxy-1-isopropyl-3-(o-toly1)-1,2,3,4- |
| | tetrahydroquinolin-7-yl)-5-(hydroxymethyl)-2,4-dihydro-3H-1,2,4-triazol-3-one; |
| 18 | 4-Ethyl-2-((3R*,4S*)-6-fluoro-4-hydroxy-1-isopropyl-3-(o-toly1)-1,2,3,4- |
| | tetrahydroquinolin-7-yl)-5-(hydroxymethyl)-2,4-dihydro-3H-1,2,4-triazol-3-one; |
| 19 | Racemic 7-(4-Ethyl-3-(hydroxymethyl)-5-oxo-4,5-dihydro-1H-1,2,4-triazol-1- |
| | y1)-6-fluoro-1-isopropyl-3-methyl-3-(o-toly1)-2,3-dihydroquinolin-4(1H)-one; |
| 20 | Racemic 4-Ethyl-2-(6-fluoro-4-hydroxy-1-isopropyl-4-methyl-3-(o-toly1)- |
| | 1,2,3,4-tetrahydroquinolin-7-yl)-5-(hydroxymethyl)-2,4-dihydro-3H-1,2,4- |
| | triazol-3-one; |
| 21 | 4-Ethyl-2-(6-fluoro-1-isopropyl-4-methyl-3-(o-tolyl)-1,2-dihydroquinolin-7-yl)- |
| | 5-(hydroxymethyl)-2,4-dihydro-3H-1,2,4-triazol-3-one; |
| 22 | 4-Ethyl-2-((2R*,4S*)-7-fluoro-4-isopropyl-2-(o-toly1)-1,2,3,4- |
| | tetrahydroquinolin-6-y1)-5-(hydroxymethyl)-2,4-dihydro-3H-1,2,4-triazol-3-one; |
| 23 | 4-Ethyl-2-((2R*,4S*)-7-fluoro-4-isopropyl-2-(o-tolyl)-1,2,3,4- |
| | tetrahydroquinolin-6-yl)-5-(hydroxymethyl)-2,4-dihydro-3H-1,2,4-triazol-3-one; |
| | and |
| 24 | 1-(1-Acetyl-7-fluoro-4-isopropyl-2-(o-toly1)-1,2,3,4-tetrahydroquinolin-6-y1)-4- |
| | ethyl-3-(hydroxymethyl)-1H-1,2,4-triazol-5(4H)-one. |

An additional embodiment of the invention is a compound of Formula (I) having the Formula (IA):

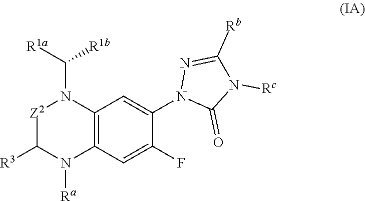

wherein
Z² is CH₂ or C═O;
R^{1a} is C₁₋₄alkyl;
R^{1b} is C₁₋₄alkyl or C₁₋₄haloalkyl;
R^{a} is H, C₁₋₆alkyl substituted with OH; CH₂(C═O)NH₂, (C═O)CH₃, and (C═O)NHCH₃;
R^{b} is C₁₋₄alkyl substituted with a member selected from the group consisting of: OH, halo, CN, OC₁₋₄alkyl, OC₁₋₄haloalkyl and OC₃₋₆cycloalkyl;
R^{c} is selected from the group consisting of: C₁₋₄alkyl, C₁₋₄haloalkyl, and C₃₋₆cycloalkyl; and
R³ is

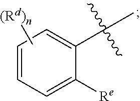

R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl; and
n is 1, or 2.

An additional embodiment of the invention is a compound of Formula (I) having the Formula (IB):

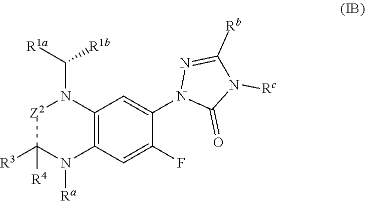

wherein
when Z² is [symbol] CH, is a double bond and R⁴ is absent; when Z² is CH₂, [symbol] is a single [symbol] bond and R⁴ is H or CH₃;
R^{1a} is C₁₋₄alkyl;
R^{1b} is C₁₋₄alkyl or C₁₋₄haloalkyl;
R^{b} is C₁₋₄alkyl substituted with OH, halo, CN, OC₁₋₄alkyl, OC₁₋₄haloalkyl or OC₃₋₆cycloalkyl;
R^{c} is C₁₋₄alkyl, C₁₋₄haloalkyl, or C₃₋₆cycloalkyl; and
R³ is

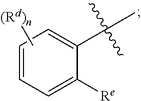

wherein
   R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
   R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl; and
n is 1, or 2.

An additional embodiment of the invention is a compound of Formula (I) having the Formula (IC):

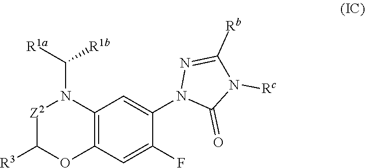

wherein
Z² is CH₂, or C═O;
R^{1a} is C₁₋₄alkyl;
R^{1b} is C₁₋₄alkyl or C₁₋₄haloalkyl;
R^{b} is C₁₋₄alkyl substituted with OH, halo, CN, OC₁₋₄alkyl, OC₁₋₄haloalkyl or OC₃₋₆cycloalkyl;
R^{c} is C₁₋₄alkyl, C₁₋₄haloalkyl, or C₃₋₆cycloalkyl;
R³ is

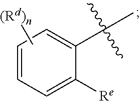

wherein
R^{d} is selected from the group consisting of: H; halo; C₁₋₆alkyl; C₁₋₆alkyl substituted with a member selected from the group consisting of OH, OCH₃, SCH₃, and OCF₃; C₁₋₆haloalkyl; C₁₋₆haloalkyl substituted with a member selected from the group consisting of OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl; and
n is 1, or 2.

An additional embodiment of the invention is a compound of Formula (I) having the Formula (ID):

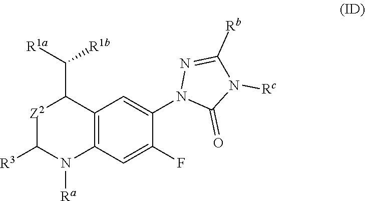

wherein
Z² is CH₂;
R^{1a} is C₁₋₄alkyl;
R^{1b} is C₁₋₄alkyl or C₁₋₄haloalkyl;
R^{a} is selected from the group consisting of H, CH₂(C═O)NH₂, (C═O)CH₃, and (C═O)NHCH₃;
R^{b} is C₁₋₄alkyl substituted with OH, halo, CN, OC₁₋₄alkyl, OC₁₋₄haloalkyl or OC₃₋₆cycloalkyl;
R^{c} is C₁₋₄alkyl, C₁₋₄haloalkyl, or C₃₋₆cycloalkyl; and
R³ is

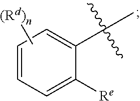

wherein
R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl; and
n is 1, or 2.

An additional embodiment of the invention is a compound of Formula (I) having the Formula (IE):

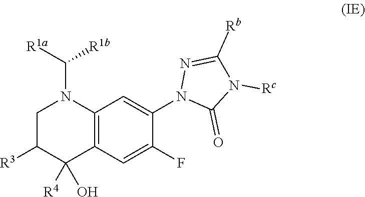

wherein
R^{1a} is C₁₋₄alkyl;
R^{1b} is C₁₋₄alkyl or C₁₋₄haloalkyl;
R^{b} is C₁₋₄alkyl substituted with OH, halo, CN, OC₁₋₄alkyl, OC₁₋₄haloalkyl or OC₃₋₆cycloalkyl;
R^{c} is C₁₋₄alkyl, C₁₋₄haloalkyl, or C₃₋₆cycloalkyl; and
R³ is

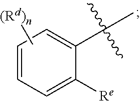

wherein
R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl;
n is 1, or 2; and
R⁴ is H or CH₃.

An additional embodiment of the invention is a compound of Formula (I) having the Formula (IA), wherein R^{1b} is CF₃.

An additional embodiment of the invention is a compound of Formula (I) having the Formula (IB), wherein R^{1a} is CH₃.

An additional embodiment of the invention is a compound of Formula (I) having the Formula (IC), wherein R^{c} is C₁₋₄alkyl.

An additional embodiment of the invention is a compound of Formula (I) having the Formula (ID), wherein R^{1a} and R^{1b} are CH₃.

An additional embodiment of the invention is a compound of Formula (I) having the Formula (IE), wherein R^{1a} and R^{1b} are CH₃.

Also within the scope of the invention are enantiomers and diastereomers of the compounds of Formula (I) (as well as Formula (IA), (IB), (IC), (ID), and (IE)). Also within the scope of the invention are pharmaceutically acceptable salts, N-oxides or solvates of the compounds of Formula (I) (as well as Formula (IA), (IB), (IC), (ID), and (IE)). Also within the scope of the invention are the pharmaceutically acceptable prodrugs of compounds of Formula (I) (as well as Formula (IA), (IB), (IC), (ID), and (IE)), and pharmaceutically active metabolites of the compounds of Formula (I) (as well as Formula (IA), (IB), (IC), (ID), and (IE)).

Also within the scope of the invention are isotopic variations of compounds of Formula (I) (as well as Formula (IA), (IB), (IC), (ID), and (IE)), such as, e.g., deuterated compounds of Formula (I). Also within the scope of the invention are the pharmaceutically acceptable salts, N-oxides or solvates of the isotopic variations of the compounds of Formula (I) (as well as Formula (IA), (IB), (IC), (ID), and (IE)). Also within the scope of the invention are the pharmaceutically acceptable prodrugs of the isotopic variations of the compounds of Formula (I) (as well as Formula (IA), (IB), (IC), (ID), and (IE)), and pharmaceutically active metabolites of the isotopic variations of the compounds of Formula (I) (as well as Formula (IA), (IB), (IC), (ID), and (IE)).

Even though the compounds of embodiments of the present invention (including their pharmaceutically acceptable salts and pharmaceutically acceptable solvates) can be administered alone, they will generally be administered in admixture with a pharmaceutically acceptable carrier, a pharmaceutically acceptable excipient and/or a pharmaceutically acceptable diluent selected with regard to the intended route of administration and standard pharmaceutical or veterinary practice.

Thus, particular embodiments of the present invention are directed to pharmaceutical and veterinary compositions comprising compounds of Formula (I) and at least one pharmaceutically acceptable carrier, pharmaceutically acceptable excipient, and/or pharmaceutically acceptable diluent. By way of example, in the pharmaceutical compositions of embodiments of the present invention, the compounds of Formula (I) may be admixed with any suitable binder(s), lubricant(s), suspending agent(s), coating agent(s), solubilizing agent(s), and combinations thereof.

An embodiment of the invention relates to a pharmaceutical composition comprising an effective amount of at least one compound selected from compounds of Formula (I), and pharmaceutically acceptable salts, isotopes, tautomers, N-oxides, solvates, and stereoisomers thereof, in accordance with any embodiment described herein; and at least one pharmaceutically acceptable excipient.

An additional embodiment of the invention is a pharmaceutical composition comprising:
(A) an effective amount of at least one compound selected from compounds of Formula (I)

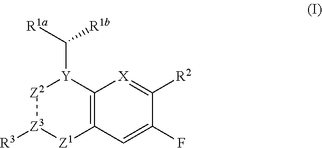

wherein
X is CH, or, optionally, N;
Y is CH or N;
Z¹ is selected from the group consisting of: CH₂, C(CH₃), CH(OH), C(CH₃)(OH), O, C═O, and NR^{a};
   R^{a} is selected from the group consisting of: H, CH₂(C═O)NH₂, (C═O)CH₃, and (C═O)NHCH₃;
Z² is CH, CH₂, or C═O;
Z³ is C, CH or C(CH₃);
each [symbol] is independently a single bond or a double bond;
   wherein
   when Z³ is CH or C(CH₃), [symbol] between Z² and Z³ is a single bond, and [symbol] between Z³ and Z¹ is a single bond;
   when Z³ is C, Z² is CH, [symbol] between Z² and Z³ is a double bond, and [symbol] between Z³ and Z¹ is a single bond;
   or
   when Z¹ is C(CH₃), [symbol] between Z² and Z³ is a single bond, and [symbol] between Z³ and Z¹ is a double bond;
R^{1a} is selected from the group consisting of: C₁₋₆alkyl; C₁₋₆alkyl substituted with OH, or OCH₃; C₁₋₆haloalkyl; C₁₋₆haloalkyl substituted with OH, or OCH₃; and C₃₋₆cycloalkyl;
R^{1b} is CH₃ or CHF₂; or R^{1a} and R^{1b} come together to form C₃₋₆cycloalkyl; C₃₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl;
R² is

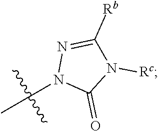

wherein
   R^{b} is C₁₋₆alkyl substituted with a member selected from the group consisting of: OH, halo, CN, OC₁₋₆alkyl, OC₁₋₆haloalkyl and OC₃₋₆cycloalkyl; and
   R^{c} is selected from the group consisting of: C₁₋₆alkyl, C₁₋₆haloalkyl, and C₃₋₆cycloalkyl;
R³ is

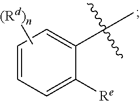

wherein
   R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
   R^{e} is selected from the group consisting of: H, halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl;
   n is 1, or 2; and
   R⁴ is H or CH₃;

or pharmaceutically acceptable salts, isotopes, tautomers, N-oxides, solvates, or stereoisomers of a compound of Formula (I);

and (B) at least one pharmaceutically acceptable excipient.

An additional embodiment of the invention is a pharmaceutical composition comprising an effective amount of a compound shown in Table 1 (e.g., a compound selected from Examples 1-24), or a pharmaceutically acceptable salt, isotope, N-oxide, solvate, or stereoisomer of the compound of Table 1, a pharmaceutically acceptable prodrug of the compound of Table 1, or a pharmaceutically active metabolite of the compound of Table 1; and at least one pharmaceutically acceptable excipient.

Solid oral dosage forms such as, tablets or capsules, containing one or more compounds of the present invention may be administered in at least one dosage form at a time, as appropriate. It is also possible to administer the compounds in sustained release formulations.

Additional oral forms in which the present inventive compounds may be administered include elixirs, solutions, syrups, and suspensions; each optionally containing flavoring agents and coloring agents.

Alternatively, one or more compounds of Formula (I) can be administered by inhalation (intratracheal or intranasal) or in the form of a suppository or pessary, or they may be applied topically in the form of a lotion, solution, cream, ointment or dusting powder. For example, they can be incorporated into a cream comprising, consisting of, and/or consisting essentially of an aqueous emulsion of polyethylene glycols or liquid paraffin. They can also be incorporated, at a concentration of between about 1% and about 10% by weight of the cream, into an ointment comprising, consisting of, and/or consisting essentially of a wax or soft paraffin base together with any stabilizers and preservatives as may be required. An alternative means of administration includes transdermal administration by using a skin or transdermal patch.

The pharmaceutical compositions of the present invention (as well as the compounds of the present invention alone) can also be injected parenterally, for example, intracavernosally, intravenously, intramuscularly, subcutaneously, intradermally, or intrathecally. In this case, the compositions will also include at least one of a suitable carrier, a suitable excipient, and a suitable diluent.

For parenteral administration, the pharmaceutical compositions of the present invention are best used in the form of a sterile aqueous solution that may contain other substances, for example, enough salts and monosaccharides to make the solution isotonic with blood.

For buccal or sublingual administration, the pharmaceutical compositions of the present invention may be administered in the form of tablets or lozenges, which can be formulated in a conventional manner.

By way of further example, pharmaceutical compositions containing at least one of the compounds of Formula (I) as the active ingredient can be prepared by mixing the compound(s) with a pharmaceutically acceptable carrier, a pharmaceutically acceptable diluent, and/or a pharmaceutically acceptable excipient according to conventional pharmaceutical compounding techniques. The carrier, excipient, and diluent may take a wide variety of forms depending upon the desired route of administration (e.g., oral, parenteral, etc.). Thus, for liquid oral preparations such as, suspensions, syrups, elixirs and solutions, suitable carriers, excipients and diluents include water, glycols, oils, alcohols, flavoring agents, preservatives, stabilizers, coloring agents and the like; for solid oral preparations such as, powders, capsules, and tablets, suitable carriers, excipients and diluents include starches, sugars, diluents, granulating agents, lubricants, binders, disintegrating agents and the like. Solid oral preparations also may be optionally coated with substances such as, sugars, or be enterically coated so as to modulate the major site of absorption and disintegration. For parenteral administration, the carrier, excipient, and diluent will usually include sterile water, and other ingredients may be added to increase solubility and preservation of the composition. Injectable suspensions or solutions may also be prepared utilizing aqueous carriers along with appropriate additives such as, solubilizers and preservatives.

According to particular embodiments, a therapeutically effective amount of a compound of Formula (I) or a pharmaceutical composition thereof may comprise a dose range from about 0.1 mg to about 3000 mg, or any particular amount or range therein, in particular from about 1 mg to about 1000 mg, or any particular amount or range therein, or, more particularly, from about 10 mg to about 500 mg, or any particular amount or range therein, of active ingredient in a regimen of about 1 to about (4×) per day for an average (70 kg) human; although, it is apparent to one skilled in the art that the therapeutically effective amount for a compound of Formula (I) will vary as will the diseases, syndromes, conditions, and disorders being treated.

For oral administration, a pharmaceutical composition may be provided in the form of one or more tablets containing about 1.0, about 10, about 50, about 100, about 150, about 200, about 250, or about 500 milligrams of a compound of Formula (I).

An embodiment of the present invention is directed to a pharmaceutical composition for oral administration, comprising a compound of Formula (I) in an amount of from about 1 mg to about 500 mg.

Advantageously, a compound of Formula (I) may be administered in a single daily dose, or the total daily dosage may be administered in divided doses of two, three and (4×) daily.

Optimal dosages of a compound of Formula (I) to be administered may be readily determined and will vary with the particular compound used, the mode of administration, the strength of the preparation, and the advancement of the disease, syndrome, condition or disorder. In addition, factors associated with the particular subject being treated, including subject gender, age, weight, diet, and time of administration, will result in the need to adjust the dose to achieve an appropriate therapeutic level and desired therapeutic effect. The above dosages are thus exemplary of the average case. There can be, of course, individual instances wherein higher or lower dosage ranges are merited, and such are within the scope of this invention.

Compounds of Formula (I) may be administered in any of the foregoing compositions and dosage regimens or by means of those compositions and dosage regimens established in the art whenever use of a compound of Formula (I) is administered to a subject in need thereof.

According to particular embodiments, one or more compounds of Formula (I) are useful in methods for treating, ameliorating and/or preventing a disease, a syndrome, a condition or a disorder that is affected by the inhibition of DHODH enzymatic activity.

An additional embodiment of the invention relates to the use of compounds of Formula (I), e.g., by inhibiting dihydroorotate oxygenase enzyme activity, in treating disorders like inflammatory disorders, autoimmune disorders, or cancer;

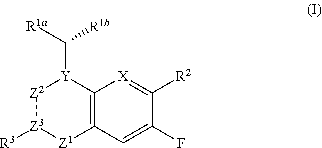

wherein
X is CH, or, optionally, N;
Y is CH or N;
Z¹ is selected from the group consisting of: CH₂, C(CH₃), CH(OH), C(CH₃)(OH), O, C═O, and NR^{a};
   R^{a} is selected from the group consisting of: H, CH₂(C═O)NH₂, (C═O)CH₃, and (C═O)NHCH₃;
Z² is CH, CH₂, or C═O;
Z³ is C, CH or C(CH₃);
each [symbol] is independently a single bond or a double bond;
   wherein
   when Z³ is CH or C(CH₃), [symbol] between Z² and Z³ is a single bond, and [symbol] between Z³ and Z¹ is a single bond;
   when Z³ is C, Z² is CH, [symbol] between Z² and Z³ is a double bond, and [symbol] between Z³ and Z¹ is a single bond;
   or
   when Z¹ is C(CH₃), [symbol] between Z² and Z³ is a single bond, and [symbol] between Z³ and Z¹ is a double bond;
R^{1a} is selected from the group consisting of: C₁₋₆alkyl; C₁₋₆alkyl substituted with OH, or OCH₃; C₁₋₆haloalkyl; C₁₋₆haloalkyl substituted with OH, or OCH₃; and C₃₋₆cycloalkyl;
R^{1b} is CH₃ or CHF₂; or R^{1a} and R^{1b} come together to form C₃₋₆cycloalkyl; C₃₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl;
R² is

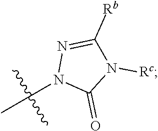

wherein
   R^{b} is C₁₋₆alkyl substituted with a member selected from the group consisting of OH, halo, CN, OC₁₋₆alkyl, OC₁₋₆haloalkyl and OC₃₋₆cycloalkyl; and
   R^{c} is selected from the group consisting of C₁₋₆alkyl, C₁₋₆haloalkyl, and C₃₋₆cycloalkyl;
R³ is

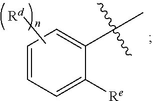

wherein
   R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of OH, and OCH₃; N(CH₃)₂; OH; CN and OC₁₋₆alkyl;
   R^{e} is selected from the group consisting of H, halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of OH, and OCH₃; OH; OC₁₋₆alkyl; and C₃₋₆cycloalkyl;
   n is 1, or 2; and
   R⁴ is H or CH₃;
      or pharmaceutically acceptable salts, isotopes, tautomers, N-oxides, solvates, or stereoisomers thereof.

In a further aspect the present invention provides a method for inhibiting or altering Dihydroorotate Dehydrogenase (DHODH) enzymatic activity, the method comprising contacting DHODH with any compound of Formula (I), aspect or embodiment disclosed herein, thereby inhibiting or otherwise altering DHODH enzymatic activity.

An additional embodiment of the present invention provides methods for treating diseases, disorders, or medical conditions mediated or otherwise affected by dihydroorotate dehydrogenase (DHODH) enzyme activity comprising administering a compound of Formula (I) to a subject in need thereof.

As used herein, the term “DHODH inhibitor” may refer to an agent that inhibits or reduces DHODH activity.

In one embodiment, the term “therapeutically effective amount” (or “effective amount”) refers to the amount of a compound of the present invention that, when administered to a subject, is effective to (1) at least partially alleviate, inhibit, prevent, and/or ameliorate a condition, or a disorder or a disease (i) mediated by DHODH enzymatic activity; or (ii) associated with DHODH enzymatic activity; or (iii) characterized by activity (normal or abnormal) of DHODH enzyme; or (2) reduce or inhibit the activity of DHODH enzyme; or (3) reduce or inhibit the expression of DHODH; or (4) modify the protein levels of DHODH. Without being bound by a particular theory, DHODH inhibitors are believed to act by inhibiting nucleic acid synthesis, cell cycle arrest or altering post-translational glycosylation of proteins involved in regulating myeloid differentiation within progenitor tumor cells.

An additional embodiment of the invention is a method of treating a subject suffering from or diagnosed with a disease, disorder, or medical condition mediated or otherwise affected by DHODH enzymatic activity, comprising administering to a subject in need of such treatment an effective amount of at least one compound selected from compounds of Formula (I) (as well as Formula (IA), (IB), (IC), (ID), and (IE), such as a compound of Table 1), enantiomers and diastereomers of the compounds of Formula (I) (as well as Formula (IA), (IB), (IC), (ID), and (IE), such as a compound of Table 1), isotopic variations of the compounds of Formula (I) (as well as Formula (IA), (IB), (IC), (ID), and (IE), such as a compound of Table 1), and pharmaceutically acceptable salts of all of the foregoing. Stated another way, according to an embodiment, a method of treating a subject suffering from or diagnosed with a disease, disorder, or medical condition comprises inhibiting or otherwise altering dihydroorotate oxygenase enzyme activity in the subject by administering to the subject an effective amount of at least one compound selected from compounds of Formula (I) (as well as Formula (IA), (IB), (IC), (ID), and (IE), such as a compound of Table 1).

In another embodiment, inhibitors of DHODH of the present invention may be used for the treatment of immunological diseases including, but not limited to, autoimmune and inflammatory disorders, e.g. arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjogren's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including edema, embolism, fibrosis, sarcoidosis, hypertension and emphysema, silicosis, respiratory failure, acute respiratory distress syndrome, BENTA disease, berylliosis, and polymyositis.

As used herein, unless otherwise noted, the term “affect” or “affected” (when referring to a disease, disorder, or medical condition that is affected by the inhibition or alteration of DHODH enzymatic activity) includes a reduction in the frequency and/or severity of one or more symptoms or manifestations of said disease, syndrome, condition or disorder; and/or includes the prevention of the development of one or more symptoms or manifestations of said disease, syndrome, condition or disorder or the development of the disease, condition, syndrome or disorder.

An additional embodiment of the invention provides a method of treatment of cancer comprising administering to a subject in need thereof, a therapeutically effective amount of a compound of Formula (I), or a pharmaceutically acceptable salt, isotope, N-oxide, solvate, or stereoisomer thereof.

According to an embodiment, the cancer is selected from but not limited to, lymphomas, leukemias, carcinomas, and sarcomas.

An additional embodiment of the invention provides the use of a compound of Formula (I), or a pharmaceutically acceptable salt, isotope, N-oxide, solvate, or stereoisomer thereof, for the treatment of one or more cancer types.

According to particular embodiments, the uses and methods of treatment described herein are directed to the treatment of cancer, wherein the cancer is selected from but not limited to:
leukemias including but not limited to acute lymphoblastic leukemia (ALL), acute myeloid leukemia (AML), (acute) T-cell leukemia, acute monocytic leukemia, acute promyelocytic leukemia (APL), bisphenotypic B myelomonocytic leukemia, chronic myeloid leukemia (CML), chronic myelomonocytic leukemia (CMML), large granular lymphocytic leukemia, plasma cell leukemia, and also myelodysplastic syndrome (MDS), which can develop into an acute myeloid leukemia,
lymphomas including but not limited to AIDS-related lymphoma, Hodgkin lymphoma, non-Hodgkin's lymphoma (NHL), T-non-Hodgkin lymphoma (T-NHL), subtypes of NHL such as Diffuse Large Cell Lymphoma (DLBCL), activated B-cell DLBCL, germinal center B-cell DLBCL, double-hit lymphoma and double-expressor lymphoma; anaplastic large cell lymphoma, marginal B cell lymphoma and primary mediastinal B-cell lymphoma, immunoblastic large cell lymphoma, Burkitt lymphoma, follicular lymphoma, hairy cell leukemia, Hodgkin's disease, mantle cell lymphoma (MCL), lymphoplasmatic lymphoma, precursor B-lymphoblastic lymphoma, lymphoma of the central nervous system, small lymphocytic lymphoma (SLL) and chronic lymphocytic leukemia (CLL); T-cell NHL such as precursor T-lymphoblastic lymphoma/leukemia, peripheral T-cell lymphoma (PTCL), cutaneous T-cell lymphoma (CTCL), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma
sarcomas including but not limited to sarcoma of the soft tissue, gliosarcoma, osteosarcoma, malignant fibrous histiocytoma, lymphosarcoma, and rhabdomyosarcoma;
   and
other cancers, such as solid tumors, including but not limited to breast cancer, colorectal carcinoma, gastric cancer, gliosarcoma, head & neck cancer, hepatocellular carcinoma, lung cancer, multiple myeloma, neuroblastoma, ovarian cancer, pancreatic cancer, prostate cancer, renal cell carcinoma and sarcoma.

In an embodiment, cancers that may benefit from a treatment with inhibitors of DHODH of the present invention include, but are not limited to, lymphomas, leukemias, carcinomas, and sarcomas, e.g. non-Hodgkin's lymphoma, diffuse large B-cell lymphoma (DLBCL), mantle cell lymphoma (MCL), follicular lymphoma (FL), marginal zone lymphoma, T-cell lymphoma, Hodgkin's lymphoma, Burkitt's lymphoma, multiple myeloma, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor).

In another embodiment of the present invention, the compounds of the present invention may be employed in combination with one or more other medicinal agents, more particularly with one or more anti-cancer agents, e.g. chemotherapeutic, antiproliferative or immunomodulating agents, or with adjuvants in cancer therapy, e.g. immunosuppressive or anti-inflammatory agents. Additional non-limiting examples of anti-cancer agents that may be administered in combination with a compound of the present invention include biologic compounds, such as monoclonal antibodies (e.g., that mediate effector function upon binding to cancer cell-associated antigens, or block interaction of a receptor expressed on cancer cells with a soluble or cell bound ligand), bispecific antibodies that mediate immune cell redirection, etc. According to an embodiment, a method of treating cancer comprises administering an effective amount of a compound of the present invention (e.g., selected from compounds of Formula (I), such as a compound shown in Table 1, pharmaceutically acceptable salts, isotopes, tautomers, N-oxides, solvates, and stereoisomers thereof) and an effective amount of one or more additional anti-cancer agents, wherein the method comprises administering the compound of the present invention and the additional anti-cancer agent(s) either simultaneously (e.g., as part of the same pharmaceutical composition) or sequentially. According to an embodiment, a pharmaceutical composition comprises an effective amount of a compound of the present invention (e.g., selected from compounds of Formula (I), such as a compound shown in Table 1, pharmaceutically acceptable salts, isotopes, tautomers, N-oxides, solvates, and stereoisomers thereof), an effective amount of one or more additional anti-cancer agents, and optionally one or more excipients.

An additional embodiment of the invention provides the use of a compound of Formula (I), or pharmaceutically acceptable salts, isotopes, tautomers, N-oxides, solvates, or stereoisomers thereof, as part of chemotherapeutic regimens for the treatment of cancers, lymphomas and leukemias alone or in combination with classic antitumoral compounds well known by the one skilled in the art.

### General Synthetic Methods

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

Abbreviations used in the instant specification, particularly the schemes and examples, are as follows:
ACN acetonitrile
AcOH glacial acetic acid
aq. aqueous
Bn or Bzl benzyl
Boc tert-butyloxycarbonyl
conc. concentrated
DCC N,N′-dicyclohexyl-carbodiimide
DCM dichloromethane
DIPEA or DIEA diisopropyl-ethyl amine
DMA dimethylaniline
DMAP 4-dimethylaminopyridine
DME dimethoxyethane
DMF N,N-dimethylformamide
DMSO dimethylsulfoxide
EA ethyl acetate
EDCI 1-ethyl-3-(3-dimethylaminopropyl) carbodiimide
ESI electrospray ionization
EtOAc or EA ethyl acetate
EtOH ethanol
FCC Flash column chromatography
GCMS gas chromatography-mass spectrometry
h or hr(s) hour or hours
HPLC high performance liquid chromatography
KHMDS Potassium bis(trimethylsilyl)amide
LiHMDS Lithium bis(trimethylsilyl)amide
MeOH methanol
MHz megahertz
min minute or minutes
MS mass spectrometry
NaHMDS Sodium bis(trimethylsilyl)amide
NMR nuclear magnetic resonance
Pd-118 [1,1′-Bis(di-tert-butylphosphino)ferrocene]dichloropalladium(II)
PE petroleum ether
RP reverse-phase
rt or RT room temperature
Rₜ retention time
Sec second or seconds
TBDPS tert-Butyldiphenylchlorosilane
TBAF tetrabutylammonium fluoride
TBS tert-Butyldimethylsilyl
TES triethylsilane
TIPS triisopropylsilane
TEA or Et₃N triethylamine
TFA trifluoroacetic acid
THF tetrahydrofuran
TLC thin layer chromatography

### PREPARATIVE EXAMPLES

Exemplary compounds useful in methods of the invention will now be described by reference to the illustrative synthetic schemes for their general preparation below and the specific examples to follow.

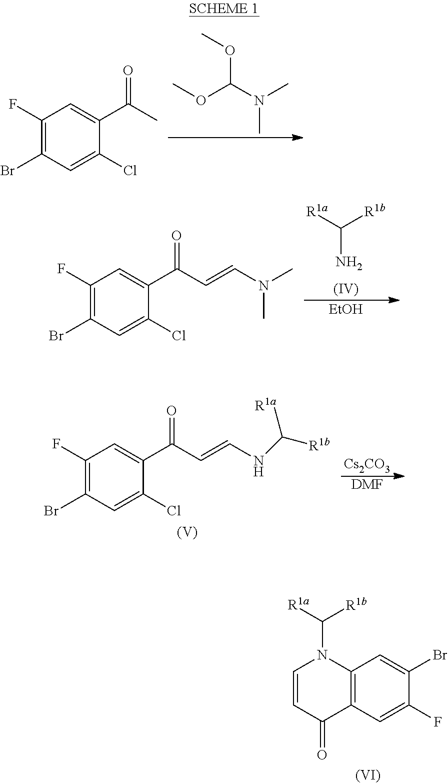

According to SCHEME 1, 1-(4-bromo-2-chloro-5-fluorophenyl)ethan-1-one is reacted in the presence of N,N-dimethyl acetal; in a suitable solvent such as toluene, DMF, acetonitrile, N,N-dimethyl acetal or the like; at temperatures ranging from such as 100° C. to 200° C., preferably 160° C.; to provide (E)-1-(4-bromo-2-chloro-5-fluorophenyl)-3-(dimethylamino)prop-2-en-1-one. (E)-1-(4-Bromo-2-chloro-5-fluorophenyl)-3-(dimethylamino)prop-2-en-1-one is reacted with an amine compound of formula (IV), where R^{1a} and R^{1b} are C₁₋₆alkyl; in an alcoholic solvent such as EtOH or a polar aprotic solvent such as DMSO or DMF; at a temperature of 110° C.; to afford a compound of the general formula (V). A compound of the formula (V) is reacted in the presence of a suitable base such as K₂CO₃, Cs₂CO₃, NaHCO₃, triethylamine, and the like; in a suitable solvent such as dimethylsulfoxide (DMSO), DMF, THF, MeCN, and the like; to afford a quinolone of formula (VI).

Compounds of formula (VI), may be made in a manner as described above; employing a commercially available or synthetically accessible amine compound of formula (IV), where R^{1a} is selected from the group consisting of: C₁₋₆alkyl substituted with OH, or OCH₃; C₁₋₆haloalkyl; C₁₋₆haloalkyl substituted with OH, or OCH₃; and C₃₋₆cycloalkyl; and R^{1b} is CH₃ or CHF₂; or R^{1a} and R^{1b} come together to form C₃₋₆cycloalkyl; C₃₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl.

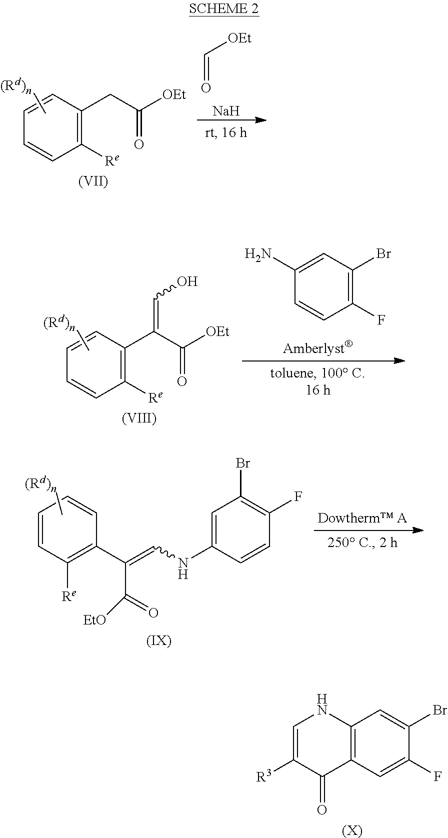

According to SCHEME 2, an ester compound of formula (VII) where n, R^{d} and R^{e} are as defined in claim 1, is reacted with ethyl formate; in the presence of a suitable base such as NaH, LiHMDS, NaHMDS, NaHMDS, and the like; in a suitable solvent such as ethyl formate, DMF, ACN, THF, and the like; at temperatures ranging from room temperature to 80° C.; for a period of about 18 hrs; to afford ester compound of formula (VIII). A compound of formula (VIII) is reacted with commercially available 3-bromo-4-fluoroaniline in the presence of a suitable base such as Amberlyst® resin, LiHMDS, NaHMDS, KHMDS, and the like; in a suitable solvent such as toluene, DMF, THF, ACN, and the like; at temperatures ranging from room temperature to 110° C.; for a period of 16 hrs; to afford a phenylaminoacrylate compound of formula (IX). A compound of formula (IX) is reacted in the presence of acid such as Dowtherm™ A, HCl, AcOH and the like; at temperatures ranging from 80° C. to 250° C.; for a period of about 2 hr; to afford a quinolone compound of formula (X).

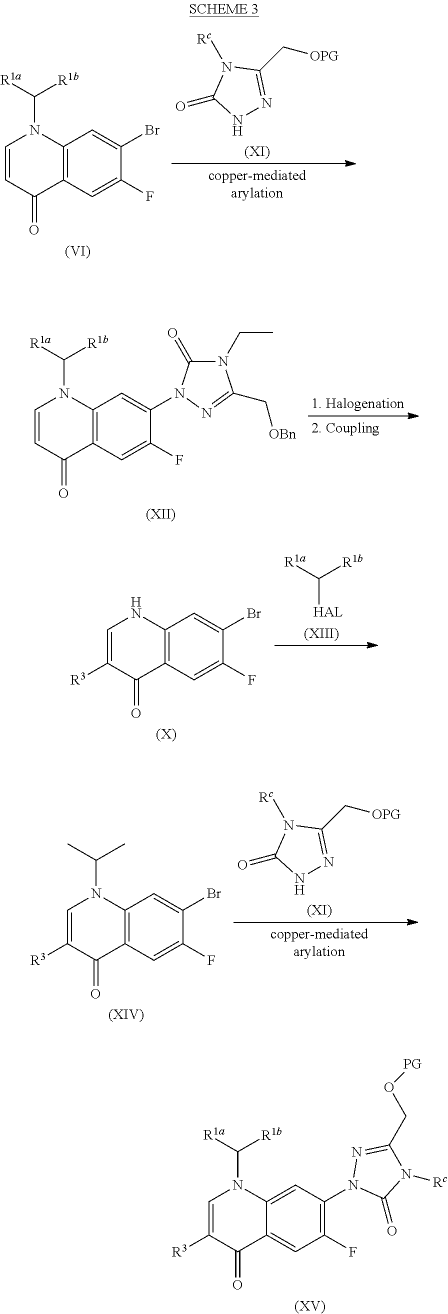

According to SCHEME 3, a compound of formula (VI), where R^{1a} and R^{1b} are as defined in claim 1; and triazolone of formula (XI), where R^{c} is C₁₋₆alkyl, and PG is a protecting group such as benzyl, para-methoxy benzyl, TBDPS, TIPS, TBS, and the like, are reacted employing copper catalyzed arylation conditions to provide a compound of formula (XII). For example, reaction of compound of formula (VI) with a compound of formula (XI); in the presence of a Cu(I) or Cu(II) salt such as CuI; with or without an additive such as KI; a ligand such as trans-N,N′-dimethylcyclohexane-1,2-diamine, N,N′-dimethylglycine, 2-((2,6-Dimethylphenyl)amino)-2-oxoacetic acid, and the like; a base such as Cs₂CO₃, K₂CO₃, K₃PO₄, K₂HPO₄, KHCO₃, Na₂CO₃, NaHCO₃, and the like; in a suitable solvent such as dioxane, and the like; at temperatures ranging from 90 to 110° C.; for a period of about 16 to 24 hours; provides a compound of formula (XII). A compound of formula (XII) is halogenated in the presence of N-bromosuccinimide (NBS), N-chlorosuccinimide (NCS), and the like; in a suitable solvent such as DMF, MeCN, THF and the like; at temperatures ranging from 0° C. to 50° C.; for a period of 1 hr to 16 hr, preferably 1 hr; to afford a haloquinoline. The resulting haloquinoline compound is reacted in a metal mediated cross coupling reaction to provide a compound of formula (XV). For example, haloquinoline compound is reacted with a suitably substituted aryl boronic acid, boronate ester, and the like; in the presence of a palladium catalyst such as PdCl₂(dtbpf), Pd(PPh₃)₄, bis(triphenylphosphine)palladium(II)chloride (PdCl₂(PPh₃)₂), bis(diphenylphosphino)ferrocene]dichloropalladium(II) complex with dichloromethane, (2-dicyclohexylphosphino-2′,6′-diisopropoxy-1,1′-biphenyl)[2-(2′-amino-1,1′-biphenyl)]palladium(II) methanesulfonate (RuPhos Pd G3), [1,1′-bis(diphenylphosphino)ferrocene]dichloropalladium(II) (Pd(dppf)Cl₂), and the like; a suitable base such as Cs₂CO₃, K₂CO₃, K₃PO₄, K₂HPO₄, KHCO₃, Na₂CO₃, NaHCO₃, and the like; in a suitable solvent such as 1,4-dioxane, DMF, acetonitrile (ACN), water, or a mixture thereof; at a temperature ranging from 50 to 80° C.; for a period of about 16 to 24 hours; to afford a compound of formula (XV).

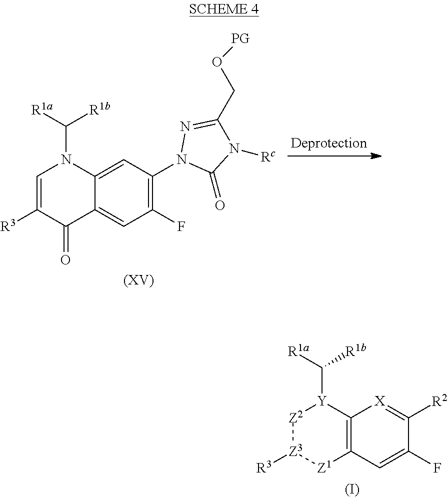

According to SCHEME 4, a compound of formula (XV), where PG is a alcohol protecting group as defined above; is deprotected employing conditions known to those skilled in the art (Greene, Protecting Groups in Organic Synthesis; John Wiley & Sons) to afford a compound of Formula (I) where R^{1a}, R^{1b}, R^{c}, and R³ are defined in claim 1, Z² is defined as C—H, [symbol] is a double bond; and Z¹ is C═O. For example, when PG is benzyl, deprotection of (XV) is achieved by treatment with neat TFA at 60° C. for 18 hrs or by treatment with BCl₃ in DCM at reduced temperatures such as 0° C. for 1 to 4 hrs or by treatment with hydrogen gas in the presence of catalytic palladium on carbon in a solvent such as EtOH, EtOAc, or the like, at room temperature of rt to afford the compound of Formula (I).

A compound of Formula (I), where [symbol] is a double bond; is reduced in the presence of a suitable reductant such as lithium aluminum hydride, and the like; in a suitable solvent such as THF, and the like; at temperatures ranging from −78° C. to 0° C.; for a period of 1 to 6 hrs; to afford a compound of Formula (I) where X is CH, R^{1a}, R^{1b}, R^{c}, and R³ are defined in claim 1, Z² is defined as CH₂, [symbol] is a single bond; and Z¹ is defined as C═O.

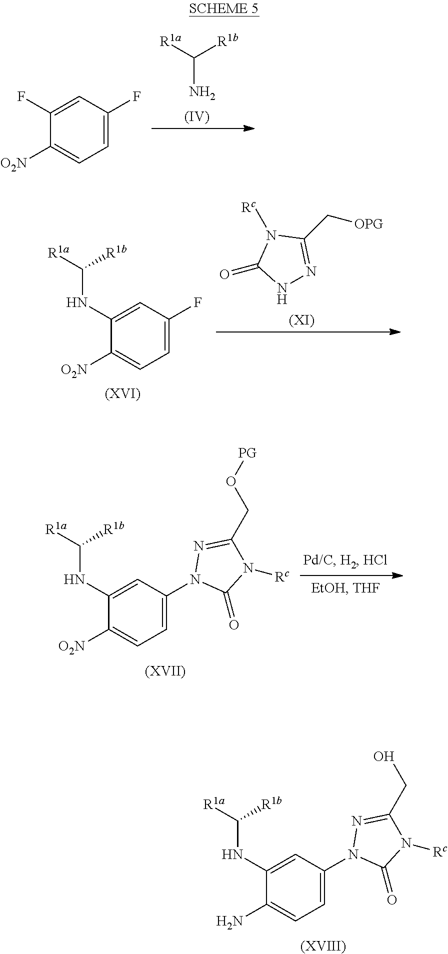

According to SCHEME 5, 2,4-difluoro-1-nitrobenzene is reacted with an amine compound of formula (IV), where R^{1a} and R^{1b} are each independently C₁₋₆alkyl and C₁₋₆haloalkyl; in the presence of a suitable base such as K₂CO₃, Cs₂CO₃, NaHCO₃, triethylamine, and the like; in a suitable solvent such as DMSO, DMF, THF, MeCN, and the like; at temperatures ranging from 80° C. to 100° C., for a period of 18 to 51 h, to afford an aniline of formula (XVI). In a similar fashion, compounds of formula (XVI) may be made in a manner as described above; employing a commercially available or synthetically accessible amine compound of formula (IV), where R^{1a} is selected from the group consisting of: C₁₋₆alkyl substituted with OH, or OCH₃; C₁₋₆haloalkyl; C₁₋₆haloalkyl substituted with OH, or OCH₃; and C₃₋₆cycloalkyl; and R^{1b} is CH₃ or CHF₂; or R^{1a} and R^{1b} come together to form C₃₋₆cycloalkyl; C₃₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl.

A compound of formula (XVI) is reacted with a triazolone compound of formula (XI) where R^{c} is C₁₋₆alkyl, and PG is a suitable protecting group as defined above; employing SNAr (addition-elimination) conditions known to one skilled in the art; using a suitable base such as K₂CO₃, Cs₂CO₃, NaHCO₃, triethylamine, and the like, in a suitable solvent such as DMF, DMSO, MeCN, THF, and the like; at temperatures ranging from 60° C. to 100° C.; for a period of 4 to 18 hr; to afford diamino substituted phenyl compound of formula (XVII). Cleavage of protecting group (PG) of a compound of formula (XVII), is achieved according to procedures known to one skilled in the art and employing established methodologies, such as those described in T. W. Greene and P. G. M. Wuts, “Protective Groups in Organic Synthesis,” 3 ed., John Wiley & Sons, 1999. For example, when PG is benzyl, deprotection is achieved employing Pd/C; under an H²; in a suitable solvent such as EtOH, MeOH, EtOAc, or a mixture thereof, preferably EtOH; with or without the presence HCl, for a period of 4 to 72 hrs, to provide a compound of formula (XVIII). Additionally, when PG is benzyl, deprotection using trifluoroacetic acid as solvent may be employed.

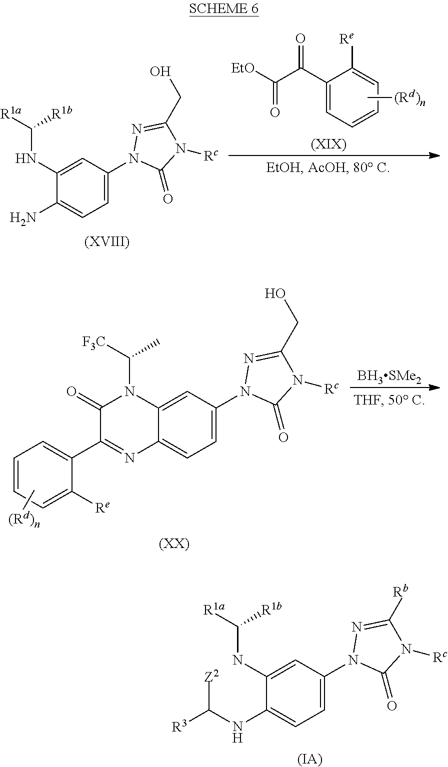

According to SCHEME 6, a diamino substituted phenyl compound of formula (XVIII), is reacted with a ketoester compound of formula (XIX), where R^{d}, n, and R^{e} are defined in claim 1; in the presence of a suitable acid such as acetic acid, trifluoroacetic acid, HCl, p-toluenesulfonic acid (PTSA or pTsOH), or H₂SO₄; in a suitable solvent such as EtOH, THF, DMF, MeCN, and the like; at temperatures ranging from 60° C. to 100° C.; for a period of 12 to 24 hrs; to afford a quinoxalinone compound of formula (XX). A compound of the formula (XX) is reduced by addition of a suitable reductant such as BH₃, LAH, DIBAL, and the like; in a suitable solvent such as THF, dioxane, and the like; at temperatures ranging from rt to 100° C.; for a period of 14 to 72 hrs; to afford a compound of Formula (IA) where R is C₁₋₆alkyl, Z² is C═O.

A compound of formula (XX) treated in the manner described above also affords a compound of Formula (IA) where R^{c} is C₁₋₆alkyl, Z² is CH₂.

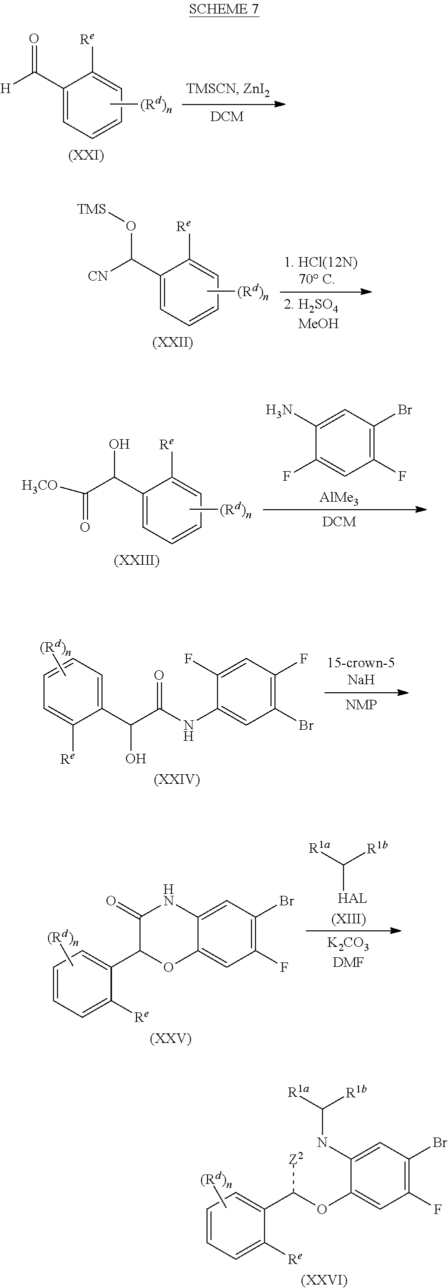

According to SCHEME 7, a ketone compound of formula (XXI), where R^{e}, R^{d}, and n are defined as in claim 1; is reacted with a cyanide source such as TMSCN, KCN, NaCN, and the like; in the presence of a Lewis acid or catalyst such as ZnI₂, Titanium(IV) isopropoxide (Ti(OiPr)₄), or N-morpholine oxide; in a suitable solvent such as DCM, THF, ether, and the like; at temperatures ranging from about 0° C. to 25° C.; for a period of 12 to 24 hrs; to afford a cyanohydrin compound of formula (XXII). A cyanohydrin compound of formula (XXII) is desilylated by treatment with an acid such as HCl, trifluoroacetic acid, sulfuric acid, and the like; at temperatures ranging from 25° C. to 100° C.; for a period of about 12 hrs. Subsequent hydrolysis is achieved by treatment with a strong acid such as HCl, trifluoroacetic acid, sulfuric acid, and the like; in a solvent such as MeOH; at a temperature of about 65° C.; for a period of 12 to 24 hrs; to afford a hydroxy ester compound of formula (XXIII). A compound of formula (XXIII) is amidated by treatment with an aniline such as 5-bromo-2,4-difluoroaniline (that has been activated using trimethylaluminum); in a suitable solvent such as DCM or toluene; at a temperature of about 65° C.; for a period of about 12 hrs; to afford an amide compound of formula (XXIV). Cyclization of compound of formula (XXIV) is accomplished by treatment with a suitable base such as NaH, NaH/15-crown-5, K₂CO₃, Cs₂CO₃, NaHCO₃, or the Li, Na, or K salt of HMDS; in a suitable solvent such as DMF, NMP, THF, MeCN, and the like; at temperatures ranging from 100° C. to 150° C.; for a period of 8 to 12 hrs; to afford a compound of formula (XXV). A compound of formula (XXV) is alkylated by treatment with an alkyl halide of formula (XIII) where R^{1a} and R^{1b} are C₁₋₆alkyl; in the presence of a suitable base such as NaH, K₂CO₃, Cs₂CO₃, NaHCO₃, or the Li, Na, or K salt of bis(trimethylsilyl)amide, and the like; in a suitable solvent such as DMF, THF, MeCN, and the like; at a temperature of about 40° C.; for a period of 12 hrs; to afford a compound of formula (XXVI) where Z² is C═O, and [symbol] is a single bond.

A compound of formula (XXVI), where Z² is C═O, is converted to compound of formula (XXVI) where Z² is CH₂; by treatment with a suitable reducing agent such as borane, in a solvent such as THF, at a temperature of about 55° C., for a period of 12 to 24 hrs, followed by subsequent borane hydrolysis using MeOH.

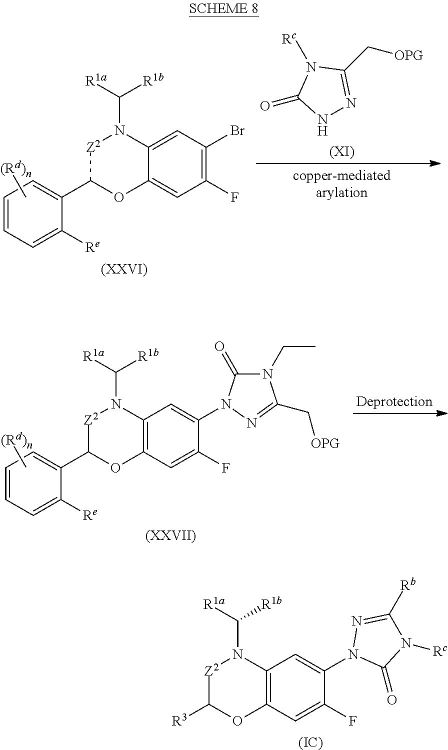

According to SCHEME 8, a compound of formula (XXVI), where Z² is CH₂ or C═O, is reacted with a compound of formula (XI), where R^{c} is C₁₋₆alkyl, employing copper mediated arylation conditions as previously described to provide a compound of formula (XXVII). Removal of the alcohol protecting group (PG) is achieved using conditions to those known in the art (Greene, Protecting Groups in Organic Synthesis; John Wiley & Sons) and those described in SCHEME 4 to afford a compound of Formula (IC) where Z² is CH₂ or C═O.

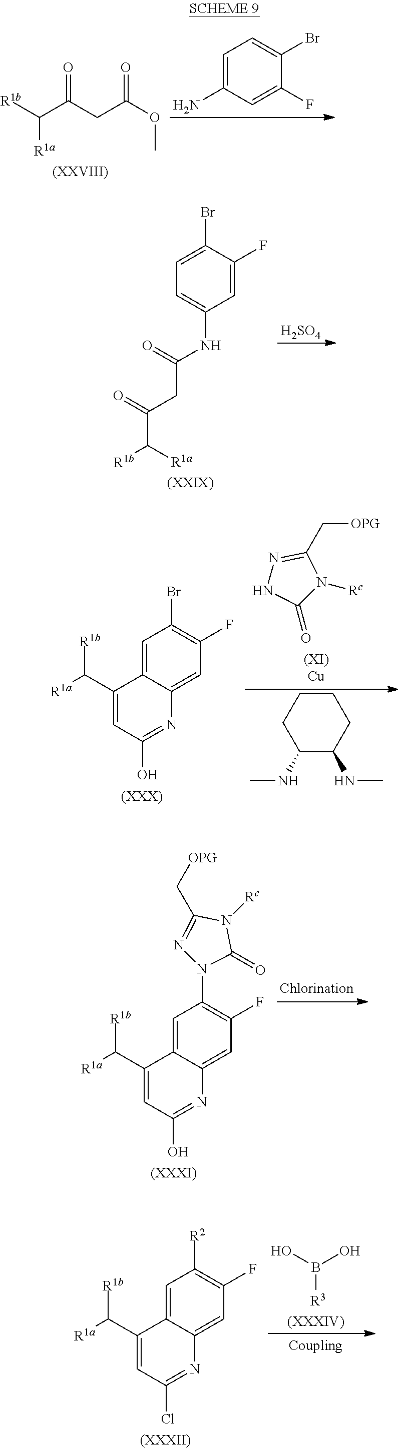

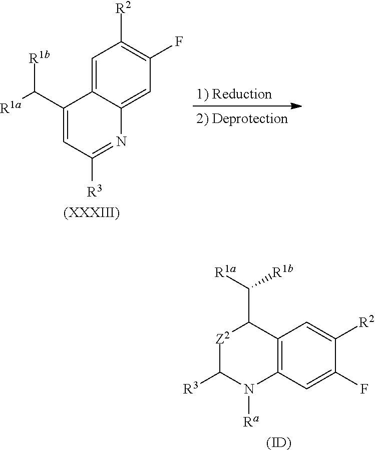

According to SCHEME 9, condensation of 4-bromo-3-fluoro aniline with a commercially available or synthetically accessible compound of formula (XXVIII), where R^{1a} and R^{1b} are as defined in claim 1; in the presence of a suitable base such as Et₃N, and the like; in a suitable solvent such as THF, toluene, ACN, and the like, preferably toluene; affords a compound of formula (XXIX). In a Knorr reaction, a compound of formula (XXIX) is cyclized employing a strong acid such as H₂SO₄, polyphosphoric acid, and the like; at temperatures ranging from 25° C. to 60° C.; for a period of 24-48 hours; to afford a hydroxy quinoline compound of formula (XXX). A compound of formula (XXX), is reacted with a commercially available or synthetically accessible compound of formula (XI), where PG is a benzyl protecting group; employing copper catalyzed arylation conditions as previously described to provide a compound of formula (XXXI). Chlorination of a compound of formula (XXXI) is achieved employing conditions known to one skilled in the art, for example, a compound of formula (XXXI) is treated with a chlorinating agent such as POCl₃, and the like, at temperatures ranging from 70-90° C., to provide a chloro-quinoline compound of formula (XXXII). A compound of formula (XXXIII) is prepared by reaction of a compound of formula (XXXII) in a metal mediated cross coupling reaction, with a commercially available or synthetically accessible appropriately substituted aryl or heteroaryl boronic acid of formula (XXXIV) (or boronate ester); employing conditions previously described to provide a compound of formula (XXXIII). Wherein when a compound of formula (XXXIII) contains a benzyl protecting group on the R² moiety, subsequent cleavage of the benzyl protecting group is achieved according to procedures known to one skilled in the art, for example, employing BBr₃, BCl₃, and the like; in a suitable solvent such as dichloromethane (DCM), and the like; at temperatures ranging from −78 to 0° C.

A compound of the formula (XXXIII) may be reduced in the presence of a suitable reductant such as sodium cyanoborohydride, sodium triacetoxyborohydride, or the like, and a suitable acid such as acetic acid, HCl, or TFA, and the like, at temperatures ranging around room temperature, for a period of 12 to 24 hrs, to afford a compound of the Formula (ID) where Z² is CH₂ and R^{a} is H. A compound of the Formula (ID) where Z² is CH₂ and R^{a} is H; may be acylated in the presence of a reagent such as acetyl chloride, acetic anhydride, or the like, in presence of DMAP, and a suitable base such as triethylamine, diisopropylethylamine, or the like, in a solvent such as DCM, THF, MeCN, or the like, at ambient temperature, for a period of 18 hrs, to afford a compound of the Formula (ID) Z² is CH₂ and R^{a} is acetyl.

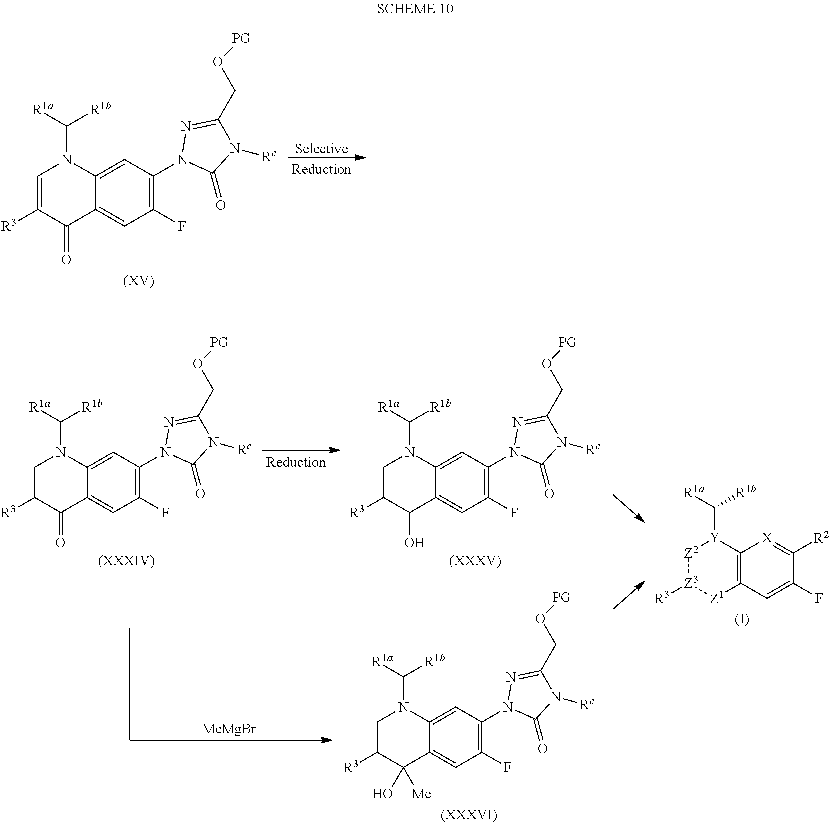

According to SCHEME 10, a compound of formula (XV), where R^{1a}, R^{1b}, R³, R^{c} are as defined in claim 1 and PG is a protecting group such as benzyl, para-methoxy benzyl, TBDPS, TIPS, TBS, is reduced with a suitable reducing agent such as lithium aluminum hydride or the like, in a suitable solvent such as THF, diethyl ether or the like, preferably THF; under controlled temperatures such as −40° C., for a time of 1 to 8 hrs, preferably 3 hrs and affords a compound of formula (XXXIV). A compound of formula (XXXIV) is further reduced under Luche conditions using a suitable Lewis acid, such as CeCl₃, a reductant, such as NaBH₄, in a suitable solvent such as MeOH, EtOH, or the like and affords a compound of formula (XXXV). Alternatively, a compound of formula (XXXIV) is reacted with MeMgBr in a solvent such as THF, diethyl ether, or the like, at a temperature ranging from −78° C. to 0° C. and affords a compound of formula (XXXVI). A compound of formula (XXXV) or a compound of formula (XXXVI), is deprotected employing conditions known to those skilled in the art (Greene, Protecting Groups in Organic Synthesis; John Wiley & Sons) to afford a compound of Formula (I) where R^{1a}, R^{1b}, R^{c}, and R³ are defined in claim 1, Z² is defined as C—H, [symbol] is a single bond; and Z¹ is C(OH)(H) or C(OH)(Me). For example, when PG is benzyl, deprotection of (XXXV) is achieved by treatment with neat TFA at 60° C. for 18 hrs or by treatment with BCl₃ in DCM at reduced temperatures such as 0° C. for 1 to 4 hrs or by treatment with hydrogen gas in the presence of catalytic palladium on carbon in a solvent such as EtOH, EtOAc, or the like, at room temperature of rt to afford the compound of Formula (I), where X is CH.

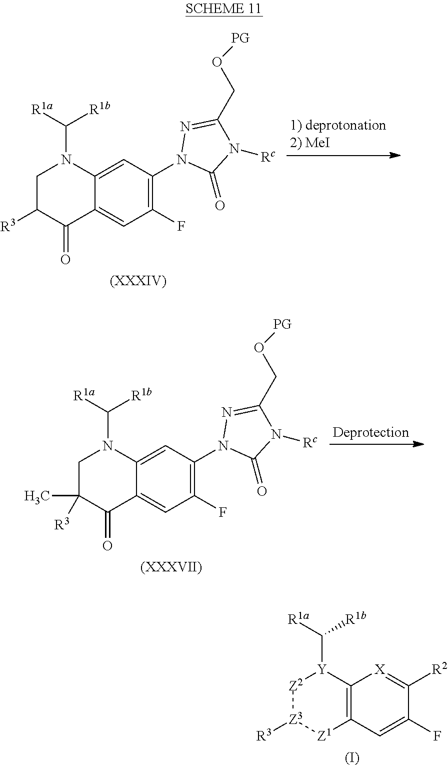

According to SCHEME 11, a compound of formula (XXXIV), where R^{1a}, R^{1b}, R³, R^{c} are as defined in claim 1 and PG is a protecting group such as benzyl, para-methoxy benzyl, TBDPS, TIPS, TBS, is alkylated by first deprotonation using a suitable base such as lithium diisopropylamide or the like and subsequent reaction with methyl iodide in a suitable solvent such as THF, diethyl ether, or the like to afford a compound of formula (XXXVII). A compound of (XXXVII) is deprotected employing conditions known to those skilled in the art (Greene, Protecting Groups in Organic Synthesis; John Wiley & Sons) to afford a compound of Formula (I) where R^{1a}, R^{1b}, R^{c}, and R³ are defined in claim 1, X is CH, Z² is defined as C—H, [symbol] is a single bond; and Z¹ is C(OH)(H) or C(OH)(Me).

Compounds of Formula (I) may be converted to their corresponding salts using methods known to one of ordinary skill in the art. For example, an amine of Formula (I) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I) may be obtained in crystalline form by recrystallization from polar solvents (including mixtures of polar solvents and aqueous mixtures of polar solvents) or from non-polar solvents (including mixtures of non-polar solvents).

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Compounds prepared according to the schemes described above may be obtained as single forms, such as single enantiomers, by form-specific synthesis, or by resolution. Compounds prepared according to the schemes above may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

The following specific examples are provided to further illustrate the invention and various preferred embodiments.

### EXAMPLES

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal-phase silica gel chromatography (FCC) was performed on silica gel (SiO₂) using prepacked cartridges.

Preparative reverse-phase high performance liquid chromatography (RP HPLC) was performed on either:
METHOD A. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (with 0.225% FA) over 10 min and then hold at 100% ACN for 2 min, at a flow rate of 25 mL/min.
or
METHOD B. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (0.1% TFA) over 10 min and then hold at 100% ACN for 2 min, at a flow rate of 25 mL/mm.
or
METHOD C. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (0.05% HCl) over 10 min and then hold at 100% ACN for 2 min, at a flow rate of 25 m/min.
or
METHOD D. a Gilson GX-281 semi-prep-HPLC with Phenomenex Gemini C18 (10 μm, 150×25 mm), AD (10 μm, 250 mm×30 mm), or Waters XBridge C18 column (5 μm, 150×30 mm), mobile phase of 0-99% ACN in water (with 0.05% ammonia hydroxide v/v) over 10 min and then hold at 100% ACN for 2 min, at a flow rate of 25 mL/min.
or
METHOD E. a Gilson GX-281 semi-prep-HPLC with Phenomenex Gemini C18 (10 μm, 150×25 mm), or Waters XBridge C18 column (5 μm, 150×30 mm), mobile phase of 5-99% ACN in water (10 mM NH₄HCO₃) over 10 min and then hold at 100% ACN for 2 min, at a flow rate of 25 mL/min.
or
METHOD F. Teledyne ISCO ACCQPrep HP150 semi-prep-HPLC with Phenomenex Gemini-NX C18 (5 μm, 150×30 mm), mobile phase of 10-100% ACN in water (10 mM NH₄OH) over 10 min and then hold at 100% ACN for 2 min, at a flow rate of 30 mL/min.

Preparative supercritical fluid high performance liquid chromatography (SFC) was performed either on a Thar 80 Prep-SFC system, or Waters 80Q Prep-SFC system from Waters. The ABPR was set to 100 bar to keep the CO₂ in SF conditions, and the flow rate may verify according to the compound characteristics, with a flow rate ranging from 50 g/min to 70 g/min. The column temperature was ambient temperature

Mass spectra (MS) were obtained on a SHIMADZU LCMS-2020 MSD or Agilent 1200\G6110A MSD using electrospray ionization (ESI) in positive mode unless otherwise indicated. Calculated (calcd.) mass corresponds to the exact mass.

Nuclear magnetic resonance (NMR) spectra were obtained on Bruker model AVIII 400 spectrometers. Definitions for multiplicity are as follows: s=singlet, d=doublet, t=triplet, q=quartet, m=multiplet, br=broad. It will be understood that for compounds comprising an exchangeable proton, said proton may or may not be visible on an NMR spectrum depending on the choice of solvent used for running the NMR spectrum and the concentration of the compound in the solution.

Chemical names were generated using ChemDraw Ultra 17.1 (CambridgeSoft Corp., Cambridge, MA) or OEMetaChem V1.4.0.4 (Open Eye).

Compounds designated as R* or S* are enantiopure compounds where the absolute configuration was not determined.

### Intermediate 1: 7-Bromo-6-fluoro-1-isopropylquinolin-4(1H)-one

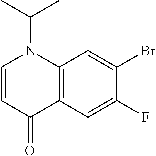 [SMILES: CC(C)n1ccc(=O)c2cc(F)c(Br)cc21]

Step A. (E)-1-(4-Bromo-2-chloro-5-fluorophenyl)-3-(dimethylamino)prop-2-en-1-one. A mixture of 1-(4-bromo-2-chloro-5-fluorophenyl)ethan-1-one (130 mg, 0.52 mmol) and N,N-dimethylformamide dimethyl acetal (1.1 mL, 8.3 mmol) was heated under microwave irradiation at 160° C. for 15 min, after which the reaction mixture was cooled to 55° C. with an air flow. The excess of DMF-DMA was removed under reduced pressure and concentrated in vacuo to give the crude title compound as an orange oil, which was used directly to the next step without further purification. MS (ESI): mass calcd. for C₁₁H₁₀BrClFNO, 306.6; m/z found, 308 [M+H]⁺.

Step B. (E)-1-(4-Bromo-2-chloro-5-fluorophenyl)-3-(isopropylamino)prop-2-en-1-one. A solution of (E)-1-(4-bromo-2-chloro-5-fluorophenyl)-3-(dimethylamino)prop-2-en-1-one (158 mg, 0.52 mmol), isopropylamine (0.22 mL, 2.6 mmol), and EtOH (1.5 mL) was heated under to microwave irradiation at 110° C. for 2 hr. The mixture was filtered and concentrated in vacuo to give the crude title compound. (170 mg) as a yellow oil, which was used directly to the next step without further purification. MS (ESI): mass calcd. for C₁₂H₁₂BrClFNO, 320.6; m/z found, 322 [M+H]⁺.

Step C. 7-Bromo-6-fluoro-1-isopropylquinolin-4(1H)-one. To a solution of (E)-1-(4-bromo-2-chloro-5-fluorophenyl)-3-(isopropylamino)prop-2-en-1-one (165 mg, 0.52 mmol) in DMF (3 mL) was added Cs₂CO₃ (503 mg, 1.54 mmol) at RT. The mixture was stirred at 100° C. for 15 h. The mixture was poured into water (10 mL) and stirred for 1 min. The aqueous phase was extracted with ethyl acetate (20 mL). The organic phase was washed with brine (10 mL), dried with anhydrous Na₂SO₄, filtered and concentrated in vacuo. The residue was purified by column chromatography (FCC, SiO₂, Ethyl acetate/MeOH=1/0 to 200/1) to give the title compound (146 mg, 0.51 mmol, 99% yield) as a yellow oil. ¹H NMR (400 MHz, CDCl₃) δ=8.18 (m, 1H), 7.81 (m, 1H), 7.69 (d, J=8.0 Hz, 1H), 6.32 (d, J=8.0 Hz, 1H), 4.78 (hept, J=6.6 Hz, 1H), 1.57 (d, J=6.6 Hz, 6H). MS (ESI): mass calcd. for C₁₂H₁₁BrFNO, 284.13; m/z found, 286 [M+H]+.

### Intermediate 2: 7-Bromo-3-(2-chloro-6-fluorophenyl)-6-fluoroquinolin-4(1H)-one

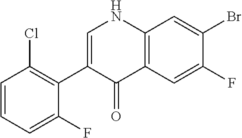 [SMILES: O=c1c(-c2c(F)cccc2Cl)c[nH]c2cc(Br)c(F)cc12]

Step A. Ethyl 2-(2-chloro-6-fluorophenyl)-3-hydroxyacrylate. To a solution of ethyl 2-(2-chloro-6-fluorophenyl)acetate (4.3 g, 20 mmol) in ethyl formate (32 mL) was added NaH (60% in mineral oil, 3.2 g). The mixture was stirred at room temperature for 16 h. The mixture was acidified to pH 3 with aqueous HCl solution (10%), then extracted with ethyl acetate. The organic extract was separated, dried over MgSO₄, filtered, and concentrated to give the crude product as an off-white solid (4.6 g, 94%), which was used directly to next step without further purification.

Step B. Ethyl 3-((3-bromo-4-fluorophenyl)amino)-2-(2-chloro-6-fluorophenyl)acrylate. To a toluene solution (45 mL) of ethyl 2-(2-chloro-6-fluorophenyl)-3-hydroxyacrylate (4.1 g, 16.8 mmol) in H²O (500 mL) and 3-bromo-4-fluoroaniline (3.2 g, 16.8 mmol) was added Amberlyst® 15 (1.5 g). The resulting reaction mixture was heated at 100° C. for 16 h. To the mixture was cooled to room temperature and filtered to remove the resin. The filtrate was concentrated under reduced pressure to give the crude product as yellow oil (6.2 g, 90%), which was used directly to next step without further purification.

Step C. 7-Bromo-3-(2-chloro-6-fluorophenyl)-6-fluoroquinolin-4(1H)-one. Ethyl 3-((3-bromo-4-fluorophenyl)amino)-2-(2-chloro-6-fluorophenyl)acrylate (6.2 g, 15 mmol) and Dowtherm™ A (6 mL) were combined and heated at 250° C. for 2 h. The mixture was cooled to room temperature, and the precipitate was collected as the first batch of product. The filtrate was concentrated to a small volume. The residue was purified by chromatography (FCC, SiO₂, 70-100% EtOAc in heptane) to afford the second batch of the desired product. The two batches were combined to give 7-bromo-3-(2-chloro-6-fluorophenyl)-6-fluoroquinolin-4(1H)-one as an off-white solid (0.96 g, 17% yield). LCMS (ESI): mass calcd. for C₁₅H₇BrClF₂NO, 368.9 m/z found, 369.9 [M+H]⁺. ¹H NMR (400 MHz, DMSO-d₆) δ12.31 (br s, 1H), 8.20 (s, 1H), 7.98 (d, J=5.87 Hz, 1H), 7.91 (d, J=8.80 Hz, 1H), 7.39-7.52 (m, 2H), 7.25-7.34 (m, 1H) ppm.

### Intermediate 3: 7-(3-((Benzyloxy)methyl)-4-ethyl-5-oxo-4,5-dihydro-1H-1,2,4-triazol-1-yl)-6-fluoro-1-isopropylquinolin-4(1H)-one

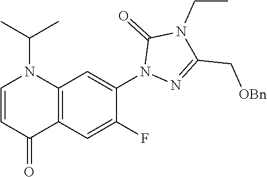 [SMILES: CCn1c(COCc2ccccc2)nn(-c2cc3c(cc2F)c(=O)ccn3C(C)C)c1=O]

Step A. 2-(Benzyloxy)acetohydrazide. To a solution of ethyl 2-(benzyloxy)acetate (55 g, 283.17 mmol) in EtOH (500 mL) was added NH₂NH₂H₂O (28.3 g, 566 mmol, 27.5 mL). The mixture was heated reflux at 78° C. stirred for 6 hr. The reaction mixture was concentrated under reduced pressure to get the title product (52 g, crude) was obtained as a colorless oil, which was used directly to next step without further purification.

Step B. 3-((Benzyloxy)methyl)-4-ethyl-1H-1,2,4-triazol-5(4H)-one. To a solution of 2-(benzyloxy)acetohydrazide (52 g, 288 mmol) in H₂O (500 mL) was added dropwise isocyanatoethane (25.1 g, 346 mmol, 27.9 mL) at 0° C. After addition, the mixture was stirred at 25° C. for 12 hr. To the mixture was added H₂O (20 mL), and an aqueous solution of NaOH (57.7 g, 1.44 mol, in 120 mL of H₂O). The mixture was stirred at 95° C. for 12 hr. The reaction mixture was quenched with HCl (12M) at 0° C. and adjusted to pH to 6. The solid was filtered and dried under reduced pressure to get the title product (61 g, 261 mmol, 91% yield) as a white solid. ¹H NMR (400 MHz, CDCl₃) δ=9.23-9.09 (m, 1H), 7.41-7.31 (m, 5H), 4.58-4.53 (m, 2H), 4.45-4.42 (m, 2H), 3.82-3.75 (m, 2H), 1.33-1.29 (m, 3H).

Step C: 7-(3-((Benzyloxy)methyl)-4-ethyl-5-oxo-4,5-dihydro-1H-1,2,4-triazol-1-yl)-6-fluoro-1-isopropylquinolin-4(1H)-one. A mixture of 7-bromo-6-fluoro-1-isopropylquinolin-4(1H)-one (Intermediate 1, 330 mg, 1.2 mmol), 5-((benzyloxy)methyl)-4-ethyl-2,4-dihydro-3H-1,2,4-triazol-3-one (395 mg, 1.7 mmol), CuI (107 mg, 0.56 mmol), Cs₂CO₃ (681.16 mg, 2.1 mmol), KI (193 mg, 1.2 mmol), trans-N,N-dimethylcyclohexane-1,2-diamine (99.13 mg, 0.7 mmol) in 1,4-dioxane (2 mL) was heated under microwave irradiation at 120° C. for 120 min, after which the reaction mixture was cooled to 55° C. with an air flow. The mixture was poured in water (10 mL) and the aqueous phase was extracted with ethyl acetate (20 mL×2). The combined organic phase was washed with brine (10 mL×2), dried with anhydrous Na₂SO₄, filtered, and concentrated in vacuo to give the crude title compound as a yellow oil, which was used directly to the next step without further purification. MS (ESI): mass calcd. for C₂₄H₂₅FN₄O₃, 436.49; m/z found, 437 [M+H]⁺.

### Intermediate 4: (S)-7-(4-Ethyl-3-(hydroxymethyl)-5-oxo-4,5-dihydro-1H-1,2,4-triazol-1-yl)-3-(2-(methylthio)phenyl)-1-(1,1,1-trifluoropropan-2-yl)quinoxalin-2(1H)-one

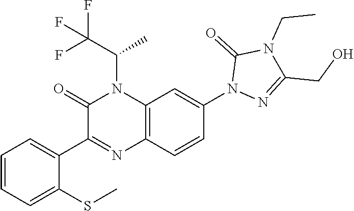 [SMILES: CCn1c(CO)nn(-c2ccc3nc(-c4ccccc4SC)c(=O)n([C@@H](C)C(F)(F)F)c3c2)c1=O]

Step A: (S)-5-Fluoro-2-nitro-N-(1,1,1-trifluoropropan-2-yl)aniline. A mixture of 2,4-difluoronitrobenzene (840 mg, 5.28 mmol), (S)-2-amino-1,1,1-trifluoropropane (601 mg, 5.31 mmol), and DIPEA (2.73 mL, 15.8 mmol) in DMF (10 mL) was heated at 130° C. for 51 h. DMF was removed in vacuo. Purification (FCC, SiO₂, 5-50% EtOAc in heptane) afforded the title compound as a yellow solid (670 mg, 50%). ¹H NMR (400 MHz, CDCl₃) δ=8.38-8.13 (m, 2H), 6.60 (dd, J=2.0, 11.2 Hz, 1H), 6.55-6.45 (m, 1H), 4.26-4.07 (m, 1H), 1.55 (d, J=6.8 Hz, 3H).

Step B: (S)-5-((Benzyloxy)methyl)-4-ethyl-2-(4-nitro-3-((1,1,1-trifluoropropan-2-yl)amino)phenyl)-2,4-dihydro-3H-1,2,4-triazol-3-one. A reaction mixture of (S)-5-fluoro-2-nitro-N-(1,1,1-trifluoropropan-2-yl)aniline (510 mg, 2.02 mmol), 5-((benzyloxy)methyl)-4-ethyl-2,4-dihydro-3H-1,2,4-triazol-3-one (670 mg, 2.87 mmol), and K₂CO₃ (406 mg, 2.94 mmol) in DMF (8 mL) was stirred at 80° C. for 18 h. DMF was removed in vacuo. Purification (SiO₂, 20-50% EtOAc in heptanes) afforded the title compound as a yellow solid (694 mg, 74%). MS (ESI): mass calcd. for C₂₁H₂₂F₃N₅O₄, 465.16; m/z found, 466.2 [M+H]⁺. ¹H NMR (400 MHz, CDCl₃) δ=8.35-8.19 (m, 2H), 7.86 (d, J=2.0 Hz, 1H), 7.44 (dd, J=2.0, 9.3 Hz, 1H), 7.41-7.30 (m, 5H), 4.61 (s, 2H), 4.52 (s, 2H), 4.44-4.28 (m, 1H), 3.84 (q, J=7.3 Hz, 2H), 1.54 (d, J=6.8 Hz, 3H), 1.34 (t, J=7.1 Hz, 3H).

Step C: (S)-2-(4-Amino-3-((1,1,1-trifluoropropan-2-yl)amino)phenyl)-4-ethyl-5-(hydroxymethyl)-2,4-dihydro-3H-1,2,4-triazol-3-one. A mixture of (S)-5-((benzyloxy)methyl)-4-ethyl-2-(4-nitro-3-((1,1,1-trifluoropropan-2-yl)amino)phenyl)-2,4-dihydro-3H-1,2,4-triazol-3-one (961 mg, 2.06 mmol), 10% Pd/C (110 mg, 0.100 mmol) and 4M HCl in dioxane (0.52 mL, 2.06 mmol) in EtOH (20 mL) and THF (5 mL) was shaken under 45 psi of H² for 21 h. The reaction was filtered, concentrated, and the residue was partitioned between EtOAc and NaHCO₃ aqueous solution. The organic phase was dried over Na₂SO₄, filtered, and concentrated under reduced pressure. Purification (FCC, SiO₂, 50-100% EtOAc in heptanes) afforded the title compound as a black solid (610 mg, 86%). MS (ESI): mass calcd. for C₁₄H₁₈F₃N₅O₂, 345.14; m/z found, 346.2 [M+H]⁺.

Step D: (S)-7-(4-Ethyl-3-(hydroxymethyl)-5-oxo-4,5-dihydro-1H-1,2,4-triazol-1-yl)-3-(2-(methylthio)phenyl)-1-(1,1,1-trifluoropropan-2-yl)quinoxalin-2(1H)-one. A reaction mixture of (S)-2-(4-amino-3-((1,1,1-trifluoropropan-2-yl)amino)phenyl)-4-ethyl-5-(hydroxymethyl)-2,4-dihydro-3H-1,2,4-triazol-3-one (50 mg, 0.14 mmol) and ethyl 2-(2-(methylthio)phenyl)-2-oxoacetate (50 mg, 0.22 mmol) in AcOH (0.50 ml, 8.7 mmol) and EtOH (0.5 mL) was purged with argon for ˜10 min and then heated at 80° C. for 16 h. The reaction mixture was cooled then concentrated under reduced pressure. The resulting product was dissolved in acetone, and Na₂CO₃ was added to neutralize any remaining AcOH. The resulting reaction mixture was concentrated under reduced pressure. Purification (FCC, SiO₂, 15-100% EtOAc in heptanes) and prep-HPLC (Luna C18 100×30 mm×5 uM column, 5-95% CH₃CN in H₂O, 0.1% TFA) afforded the title compound as a TFA salt. The HPLC compound fractions were concentrated under reduced pressure and the resulting product was partitioned between DCM and saturated NaHCO₃ aqueous solution. The organic layer was dried over Na₂SO₄, filtered, and concentrated to afford the title compound as a yellow solid (37 mg, 50%). MS (ESI): mass calcd. for C₂₃H₂₂F₃N₅O₃S, 505.14; m/z found, 506.1 [M+H]⁺. ¹H NMR (400 MHz, CDCl₃) δ=8.67 (s, 0.7H), 8.32 (s, 0.3H), 8.01-7.87 (m, 2H), 7.61-7.52 (m, 1H), 7.51-7.40 (m, 2H), 7.36-7.28 (m, 1H), 6.55-6.38 (m, 0.7H), 5.23-5.05 (m, 0.3H), 4.73-4.58 (m, 2H), 3.92 (q, J=7.3 Hz, 2H), 2.43 (s, 3H), 2.40-2.25 (m, 1H), 1.96 (d, J=7.8 Hz, 3H), 1.42 (t, J=7.3 Hz, 3H).

### Intermediate 5: (S)-3-(2-Chloro-6-fluorophenyl)-7-(4-ethyl-3-(hydroxymethyl)-5-oxo-4,5-dihydro-1H-1,2,4-triazol-1-yl)-1-(1,1,1-trifluoropropan-2-yl)quinoxalin-2(1H)-one

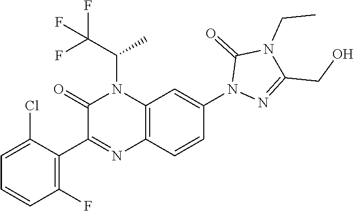 [SMILES: CCn1c(CO)nn(-c2ccc3nc(-c4c(F)cccc4Cl)c(=O)n([C@@H](C)C(F)(F)F)c3c2)c1=O]

The title compound was prepared in a manner analogous to Intermediate 4, except using ethyl 2-(2-chloro-6-fluorophenyl)-2-oxoacetate instead of ethyl 2-(2-(methylthio)phenyl)-2-oxoacetate in Step D. MS (ESI): mass calcd. for C₂₂H₁₈ClF₄N₅O₃, 511.10; m/z found, 512.1 [M+H]⁺. ¹H NMR (400 MHz, CDCl₃) δ=8.73 (s, 0.7H), 8.39 (s, 0.3H), 8.13-7.87 (m, 2H), 7.49-7.27 (m, 2H), 7.14 (t, J=8.3 Hz, 1H), 6.59-6.37 (m, 0.7H), 5.27-5.05 (m, 0.3H), 4.68 (s, 2H), 3.92 (q, J=7.0 Hz, 2H), 2.61 (br s, 1H), 1.98 (d, J=7.8 Hz, 3H), 1.42 (t, J=7.3 Hz, 3H).

### Intermediate 6: 3-(((tert-Butyldiphenylsilyl)oxy)methyl)-4-ethyl-1H-1,2,4-triazol-5(4H)-one

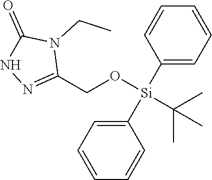 [SMILES: CCn1c(CO[Si](c2ccccc2)(c2ccccc2)C(C)(C)C)n[nH]c1=O]

Step A. 4-Ethyl-5-(hydroxymethyl)-2,4-dihydro-3H-1,2,4-triazol-3-one. To a solution of 5-[(benzyloxy)methyl]-4-methyl-2,4-dihydro-3H-1,2,4-triazol-3-one (100 mg, 0.429 mmol, 1.0 eq.) in Methanol (10 mL) was added Pd/C (10 mg). The resulting mixture was maintained under Hydrogen and stirred at rt for 6 h. The reaction was filtered and concentrated to afford the title compound, which was used without further purification. LC/MS: mass calcd. for C₅H₉N₃O₂: 143.07, found: 144.10 [M+H]⁺.

Step B. 3-(((tert-Butyldiphenylsilyl)oxy)methyl)-4-ethyl-TH-1,2,4-triazol-5(4H)-one. To a solution of 4-ethyl-5-(hydroxymethyl)-2,4-dihydro-3H-1,2,4-triazol-3-one (3000 mg, 20.9 mmol, 1.0 eq.) in N,N-Dimethylformamide (30 mL) was added tert-butylchlorodiphenylsilane (6.5 mL, 25.1 mmol, 1.2 eq.) and Imidazole (1.5 g, 23.0 mmol, 1.1 eq.). The resulting mixture was stirred at rt for overnight. The reaction mixture was quenched with water (100 mL). The resulting mixture was extracted with ethyl acetate (3×100 mL). The organic layers were combined, dried over anhydrous sodium sulfate, filtered, and concentrated. Purification by silica gel chromatography (50-80% ethyl acetate/petroleum ether) afforded the title compound as a white solid (4.9 g, 61% yield). LC/MS: mass calcd. for C₂₁H₂₇N₃O₂Si: 381.19, found: 382.15 [M+H]⁺.

### Intermediate 7: 6-Bromo-2-(2-chloro-6-fluorophenyl)-7-fluoro-4-isopropyl-2H-benzo[b][1,4]oxazin-3(4H)-one

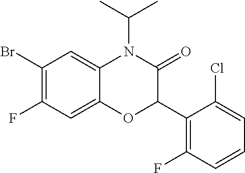 [SMILES: CC(C)N1C(=O)C(c2c(F)cccc2Cl)Oc2cc(F)c(Br)cc21]

Step A. 2-(2-Chloro-6-fluorophenyl)-2-((trimethylsilyl)oxy)acetonitrile. To a solution of 2-chloro-6-fluoro-benzaldehyde (30 g, 189.21 mmol) in DCM (300 mL) was added diiodozinc (6.04 g, 18.92 mmol), following by trimethylsilyl cyanide (37.5 g, 378.4 mmol, 47.3 mL) dropwise at 0° C. The mixture was stirred at 25° C. for 12 hrs. The reaction mixture was diluted with NaHCO₃ (400 mL, sat. aq.) and extracted with EtOAc (200 mL×3). The combined organic layers were dried over anhydrous Na₂SO₄, filtered and concentrated under reduced pressure. The residue was purified by column chromatography (FCC, SiO₂, petroleum ether/ethyl acetate=1/0 to 0/1) to give the title compound (32 g, 124 mmol, 66% yield) as yellow oil. ¹H NMR (400 MHz, CDCl₃) δ=7.25-7.10 (m, 3H), 5.87 (d, J=1.6 Hz, 1H), 0.08 (s, 9H).

Step B. 2-(2-Chloro-6-fluorophenyl)-2-hydroxyacetic acid. A solution of 2-(2-chloro-6-fluoro-phenyl)-2-trimethylsilyloxy-acetonitrile (27 g, 105 mmol) in HCl (270 mL, 12 M) was stirred at 75° C. for 12 hrs. The reaction mixture was extracted with EtOAc (300 mL×2). The combined organic layers were concentrated under reduced pressure. The crude product was diluted with sat. aq. NaHCO₃ (500 mL), and then washed with EtOAc (200 mL×2). The aqueous phase was adjusted the “pH” to 1 with HCl (1 N), and then extracted with EtOAc (200 mL×2). The combined organic layers were concentrated under reduced pressure to give the title compound (12.3 g, 59.7 mmol, 57% yield, 99.3% purity) as white solid. MS (ESI): mass calcd. for C₈H₆ClFO₃, 204.0; m/z found, 203.0 [M−H]⁺. ¹H NMR (400 MHz, DMSO-d₆) δ=7.41-7.27 (m, 1H), 7.34-7.32 (m, 1H), 7.24-7.22 (m, 1H), 5.41 (s, 1H).

Step C. Methyl 2-(2-chloro-6-fluorophenyl)-2-hydroxyacetate. To a solution of 2-(2-chloro-6-fluoro-phenyl)-2-hydroxy-acetic acid (5 g, 24 mmol) in MeOH (50 mL) was added H₂SO₄ (479 mg, 4.89 mmol, 260 μL) at 20° C. The reaction mixture was stirred at 65° C. for 7 hrs. The reaction mixture was concentrated under reduced pressure. Purification (SiO₂, petroleum ether/ethyl acetate=10/1 to 5/1) afforded the title compound (4.7 g, 21 mmol, 88% yield, 99.7% purity) as white solid. MS (ESI): mass calcd. for C₉H₈ClFO₃, 218.0; m/z found, 201.5[M−H₂O+H]⁺. ¹H NMR (400 MHz, CDCl₃) δ=7.31-7.29 (m, 1H), 7.25-7.23 (m, 1H), 7.04-7.01 (m, 1H), 5.69 (d, J=6.0 Hz, 1H), 3.83 (s, 3H), 3.58 (d, J=6.0 Hz, 1H).

Step D. N-(5-Bromo-2,4-difluorophenyl)-2-(2-chloro-6-fluorophenyl)-2-hydroxyacetamide. To a solution of 5-bromo-2,4-difluoro-aniline (9.13 g, 43.9 mmol) in DCM (30 mL) was added Al(CH₃)₃ (2 M, 29.3 mL). The reaction mixture was stirred at 15° C. for 0.5 hr. A solution of methyl 2-(2-chloro-6-fluoro-phenyl)-2-hydroxy-acetate (3.2 g, 14.6 mmol) in DCM (30 mL) was added to the reaction mixture. The resulting reaction mixture was stirred at 60° C. for 12 hrs. The reaction mixture added to iced water (300 mL), and then extracted with DCM (200 mL×2). The aqueous phase was added HCl (60 mL, 1N), and then extracted with EtOAc (100 mL×2). The combined organic layers were washed with brine (300 mL), dried over anhydrous Na₂SO₄, filtered and concentrated under reduced pressure. Purification (FCC, SiO₂, petroleum ether/ethyl acetate 1/0 to 5/1) afforded the title compound as yellow solid. MS (ESI): mass calcd. For C₁₄H₈BrClF₃NO₂, 395; m/z found, 396 [M+1]⁺. ¹H NMR (400 MHz, CDCl₃) δ=8.67-8.63 (m, 2H), 7.34-7.31 (m, 1H), 7.28-7.27 (m, 1H), 7.07-7.01 (m, 1H), 6.99-6.97 (m, 1H), 5.84 (d, J=6.0 Hz, 1H), 3.23 (d, J=6.0 Hz, 1H).

Step E. 6-Bromo-2-(2-chloro-6-fluorophenyl)-7-fluoro-2H-benzo[b][1,4]oxazin-3(4H)-one. To a solution of N-(5-bromo-2,4-difluoro-phenyl)-2-(2-chloro-6-fluoro-phenyl)-2-hydroxy-acetamide (0.500 g, 1.27 mmol) and 15-crown-5 (558 mg, 2.53 mmol, 503 μL) in N-methyl-2-pyrrolidone (NMP) (50 mL) was added NaH (101 mg, 2.53 mmol, 60% purity) at 0° C. The reaction mixture was stirred at 140° C. for 8 hrs. The reaction mixture was cooled then poured into iced water (50 mL) at 0° C., and then extracted with EtOAc (80 mL). The organic layer was washed with brine (40 mL×5), dried over anhydrous Na₂SO₄, filtered, and concentrated under reduced pressure. Purification (FCC, SiO₂, petroleum ether/ethyl acetate=30/1 to 20/1) afforded the title compound (180 mg, 450 μmol, 36% yield, 94% purity) as yellow solid. MS (ESI): mass calcd. for C₁₄H₇BrClF₂NO₂, 375; m/z found, 376 [M+1]⁺. ¹H NMR (400 MHz, CDCl₃) δ=8.78 (s, 1H), 7.40-7.38 (m, 1H), 7.32-7.30 (m, 1H), 7.12-7.07 (m, 1H), 6.98 (d, J=6.4 Hz, 1H), 6.79 (d, J=8.4 Hz, 1H), 6.18 (s, 1H).

Step F. 6-Bromo-2-(2-chloro-6-fluorophenyl)-7-fluoro-4-isopropyl-2H-benzo[b][1,4]oxazin-3(4H)-one. To a solution of 6-bromo-2-(2-chloro-6-fluoro-phenyl)-7-fluoro-4H-1,4-benzoxazin-3-one (140 mg, 350 μmol) and 2-iodopropane (119 mg, 700 μmol, 70 uL) in DMF (4 mL) was added K₂CO₃ (145 mg, 1.05 mmol). The reaction mixture was stirred at 40° C. for 12 hrs. The reaction mixture was diluted with EtOAc (30 mL) and the organics were washed with brine (40 mL×5). The combined organic layers were dried over anhydrous Na₂SO₄, filtered, and concentrated under reduced pressure. Purification (prep-TLC, SiO₂, petroleum ether/ethyl acetate=10/1) afforded the title compound (52.5 mg, 109 μmol, 87% purity) as white solid. MS (ESI): mass calcd. for C₁₇H₁₃BrClF₂NO₂, 417.0; m/z found, 418.0 [M+1]⁺. ¹H NMR (400 MHz, CDCl₃) δ=7.37-7.28 (m, 3H), 7.08-7.02 (m, 1H), 6.81 (d, J=8.4 Hz, 1H), 5.98 (s, 1H), 4.61-4.57 (m, 1H), 1.60 (dd, J=6.8 Hz, 12.0 Hz, 6H).

### Example 1: 7-(4-Ethyl-3-(hydroxymethyl)-5-oxo-4,5-dihydro-1H-1,2,4-triazol-1-yl)-6-fluoro-3-(3-fluorophenyl)-1-isopropylquinolin-4(1H)-one

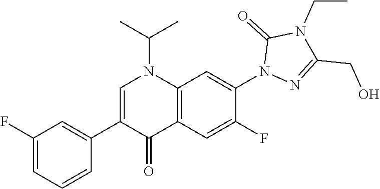 [SMILES: CCn1c(CO)nn(-c2cc3c(cc2F)c(=O)c(-c2cccc(F)c2)cn3C(C)C)c1=O]

Step A. 7-(3-((Benzyloxy)methyl)-4-ethyl-5-oxo-4,5-dihydro-1H-1,2,4-triazol-1-yl)-3-bromo-6-fluoro-1-isopropylquinolin-4(1H)-one. To a solution of 7-(3-((benzyloxy)methyl)-4-ethyl-5-oxo-4,5-dihydro-1H-1,2,4-triazol-1-yl)-6-fluoro-1-isopropylquinolin-4(1H)-one (Intermediate 3, 600 mg, 1.38 mmol) in DMF (10 mL) was added N-bromosuccinimide (NBS) (269 mg, 1.5 mmol) at 0° C. The mixture was stirred under atmosphere of N₂ at 0° C. for 1 h. The combined organic phase was washed with brine (10 mL), dried with anhydrous Na₂SO₄, filtered, and concentrated in vacuo. The residue was purified by column chromatography (FCC, SiO₂, Ethyl acetate/MeOH=1/0 to 200/1) to give the title compound (540 mg, 1.05 mmol, 76% yield) as a yellow oil. ¹H NMR (400 MHz, CDCl₃) δ=8.40-8.30 (m, 1H), 8.11 (s, 1H), 8.01 (d, J=5.8 Hz, 1H), 7.45-7.32 (m, 5H), 4.84 (hept, J=6.6 Hz, 1H), 4.63 (s, 2H), 4.54 (s, 2H), 3.88 (q, J=7.2 Hz, 2H), 1.60 (d, J=6.5 Hz, 6H), 1.38 (t, J=7.2 Hz, 3H). MS (ESI): mass calcd. for C₂₄H₂₄BrFN₄O₃, 515.38; m/z found, 517 [M+H]+.

Step B: 7-(3-((Benzyloxy)methyl)-4-ethyl-5-oxo-4,5-dihydro-1H-1,2,4-triazol-1-yl)-6-fluoro-3-(3-fluorophenyl)-1-isopropylquinolin-4(1H)-one. To a solution of 7-(3-((benzyloxy)methyl)-4-ethyl-5-oxo-4,5-dihydro-1H-1,2,4-triazol-1-yl)-3-bromo-6-fluoro-1-isopropylquinolin-4(1H)-one (40 mg, 0.078 mmol), 3-fluorophenylboronic acid (27.15 mg, 0.2 mmol) and tetrakis(triphenylphosphine) palladium (9 mg, 0.0078 mmol) in toluene (1 mL) was added MeOH (0.4 mL), and distilled water (0.11 mL) at RT under N₂. The mixture was stirred at 100° C. for 3 h. Then the reaction mixture was filtered through a short plug with silica gel, and then diluted with H₂O (10 mL) and extracted with EtOAc (10 mL×2). The combined organic layers were washed with brine (10 mL), dried over Na₂SO₄, filtered, and concentrated in vacuo to provide the title compound as a pale yellow solid (42 mg, crude), which was used directly to the next step without further purification. MS (ESI): mass calcd. for C₃₀H₂₈F₂N₄O₃, 530.57; m/z found, 531 [M+H]⁺.

Step C. 7-(4-Ethyl-3-(hydroxymethyl)-5-oxo-4,5-dihydro-1H-1,2,4-triazol-1-yl)-6-fluoro-3-(3-fluorophenyl)-1-isopropylquinolin-4(1H)-one. To a solution of 7-(3-((benzyloxy)methyl)-4-ethyl-5-oxo-4,5-dihydro-1H-1,2,4-triazol-1-yl)-6-fluoro-3-(3-fluorophenyl)-1-isopropylquinolin-4(1H)-one in EtOH (5 mL) was added 30% Pd/C (8 mg, 0.0075 mmol). The reaction mixture was hydrogenated under atmosphere of 50 psi H₂ at 25° C. for 16 h. The reaction mixture was filtered and concentrated in vacuo. The residue was purified by RP HPLC (Isco AcuuPrep, 30×100 mm, 20-100% ACN/water (10 mM NH₄OH), 15 min run time, Gemini C18 column) to provide the title compound (12 mg, 0.027 mmol, 36.14% yield) as a white solid. ¹H NMR (400 MHz, CDCl₃) δ=8.37 (d, J=11.2 Hz, 1H), 8.07 (d, J=5.8 Hz, 1H), 7.95 (s, 1H), 7.46-7.38 (m, 3H), 7.05 (t, J=7.9 Hz, 1H), 4.99-4.88 (m, 1H), 4.69 (s, 2H), 3.93 (q, J=7.2 Hz, 2H), 1.64 (d, J=6.5 Hz, 6H), 1.43 (t, J=7.2 Hz, 3H). MS (ESI): mass calcd. for C₂₃H₂₂F₂N₄O₃, 440.45; m/z found, 441.1 [M+H]+.

### Example 2: 7-(4-Ethyl-3-(hydroxymethyl)-5-oxo-4,5-dihydro-1H-1,2,4-triazol-1-yl)-3-(2-(methylthio)phenyl)-1-((S)-1,1,1-trifluoropropan-2-yl)-3,4-dihydroquinoxalin-2(1H)-one

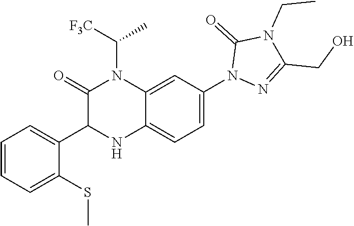 [SMILES: CCn1c(CO)nn(-c2ccc3c(c2)N([C@@H](C)C(F)(F)F)C(=O)C(c2ccccc2SC)N3)c1=O]

To a solution of (S)-7-(4-ethyl-3-(hydroxymethyl)-5-oxo-4,5-dihydro-1H-1,2,4-triazol-1-yl)-3-(2-(methylthio)phenyl)-1-(1,1,1-trifluoropropan-2-yl)quinoxalin-2(1H)-one (Intermediate 4, 16 mg, 0.032 mmol) in THF (1 mL) was added borane-methyl sulfide complex (0.017 mL, 0.18 mmol). The mixture was stirred at RT for 14 h and then heated at 50° C. for 69 h. After cooling to room temperature (RT), 2N HCl solution was added slowly, and the mixture was stirred for ˜ 15 min, and then basified with 3N NaOH. The organic layer was separated, and the aqueous layer was extracted with DCM. The combined organic layers were dried over Na₂SO₄, filtered, concentrated, and purified by RF-HPLC (Luna C18 100×30 mm×5 uM column, 5-95% CH₃CN in H₂O, 0.1% TFA). The fractions were concentrated and partitioned between DCM and saturated NaHCO₃ aqueous solution. The organic layer was dried over Na₂SO₄, filtered, and concentrated to give the title compound (5 mg, 31%). MS (ESI): mass calcd. for C₂₃H₂₄F₃N₅O₃S, 507.16; m/z found, 508.2 [M+H]⁺. ¹H NMR (400 MHz, CD₃OD) δ=7.97-7.70 (m, 1H), 7.49-7.00 (m, 4H), 7.00-6.89 (m, 1H), 6.85 (d, J=8.3 Hz, 1H), 6.00-5.70 (m, 1H), 5.60-5.30 (m, 2H), 4.58 (d, J=3.9 Hz, 2H), 3.89 (dq, J=3.2, 7.3 Hz, 2H), 2.50 (s, 1.5H), 2.45 (s, 1.5H), 1.85-1.50 (m, 3H), 1.37 (dt, J=3.2, 7.2 Hz, 3H).

### Example 3: 3-(2-Chloro-6-fluorophenyl)-7-(4-ethyl-3-(hydroxymethyl)-5-oxo-4,5-dihydro-1H-1,2,4-triazol-1-yl)-1-((S)-1,1,1-trifluoropropan-2-yl)-3,4-dihydroquinoxalin-2(1H)-one

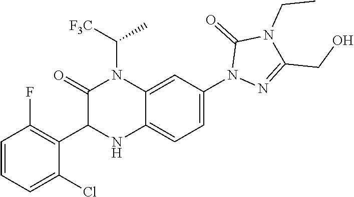 [SMILES: CCn1c(CO)nn(-c2ccc3c(c2)N([C@@H](C)C(F)(F)F)C(=O)C(c2c(F)cccc2Cl)N3)c1=O]

To a solution of (S)-3-(2-chloro-6-fluorophenyl)-7-(4-ethyl-3-(hydroxymethyl)-5-oxo-4,5-dihydro-1H-1,2,4-triazol-1-yl)-1-(1,1,1-trifluoropropan-2-yl)quinoxalin-2(1H)-one (Intermediate 5, 43 mg, 0.084 mmol) in THF (2 mL) was added borane-methyl sulfide complex (0.080 mL, 0.84 mmol). The reaction mixture was stirred at RT for 40 h, and 2N HCl solution was added slowly. LCMS indicated formation of the title compound as well as 2-(2-(2-chloro-6-fluorophenyl)-4-((S)-1,1,1-trifluoropropan-2-yl)-1,2,3,4-tetrahydroquinoxalin-6-yl)-4-ethyl-5-(hydroxymethyl)-2,4-dihydro-3H-1,2,4-triazol-3-one (Example 4). After stirring for ˜15 min, the mixture was basified with 3N NaOH. The organic layer was separated, and the aqueous layer was extracted with EtOAc. The organic layer was dried over Na₂SO₄, filtered, concentrated, and purified by flash column chromatography (12 g column, 20-70% EtOAc in heptanes) and RF-HPLC (Luna C18 100×30 mm×5 uM column, 5-95% CH₃CN in H₂O, 0.1% TFA). The fractions were concentrated and partitioned between DCM and saturated NaHCO₃ aqueous solution. The organic layer was dried over Na₂SO₄, filtered, and concentrated to give the title compound (4 mg, 9.3%). MS (ESI): mass calcd. for C₂₂H₂₀ClF₄N₅O₃, 513.12; m/z found, 514.2 [M+H]⁺. ¹H NMR (400 MHz, CDCl₃) δ=8.08-7.81 (m, 1H), 7.59-7.40 (m, 1H), 7.36-7.18 (m, 3H), 7.10-6.89 (m, 1H), 6.88-6.63 (m, 1H), 5.60 (s, 1H), 4.66 (d, J=3.9 Hz, 2H), 4.33-4.05 (m, 1H), 3.96-3.82 (m, 2H), 2.18 (br s, 1H), 1.90-1.55 (m, 3H), 1.39 (dt, J=2.0, 7.1 Hz, 3H). Additional fractions were isolated and provided 2-(2-(2-chloro-6-fluorophenyl)-4-((S)-1,1,1-trifluoropropan-2-yl)-1,2,3,4-tetrahydroquinoxalin-6-yl)-4-ethyl-5-(hydroxymethyl)-2,4-dihydro-3H-1,2,4-triazol-3-one (Example 4).

### Example 4: 2-(2-(2-Chloro-6-fluorophenyl)-4-((S)-1,1,1-trifluoropropan-2-yl)-1,2,3,4-tetrahydroquinoxalin-6-yl)-4-ethyl-5-(hydroxymethyl)-2,4-dihydro-3H-1,2,4-triazol-3-one

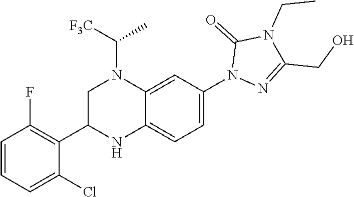 [SMILES: CCn1c(CO)nn(-c2ccc3c(c2)N([C@@H](C)C(F)(F)F)CC(c2c(F)cccc2Cl)N3)c1=O]

The title compound was isolated as a side product from Example 3 (2 mg, 5%). MS (ESI): mass calcd. for C₂₂H₂₂ClF₄N₅O₂, 499.14; m/z found, 500.3 [M+H]⁺. ¹H NMR (400 MHz, CDCl₃) δ=7.42-7.29 (m, 2H), 7.25-7.13 (m, 2H), 7.05-6.95 (m, 1H), 6.72-6.52 (m, 1H), 5.23-5.13 (m, 1H), 5.00-4.91 (m, 1H), 4.65 (s, 2H), 4.53-4.38 (m, 1H), 3.87 (q, 2H), 3.63-3.41 (m, 2H), 1.79-1.51 (m, 3H), 1.44-1.33 (m, 3H).

### Example 5: 2-(2-Chloro-6-fluorophenyl)-6-(4-ethyl-3-(hydroxymethyl)-5-oxo-4,5-dihydro-1H-1,2,4-triazol-1-yl)-7-fluoro-4-isopropyl-2H-benzo[b][1,4]oxazin-3(4H)-one

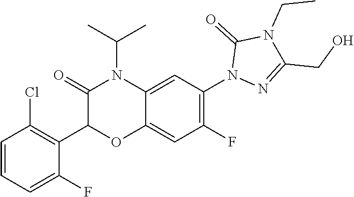 [SMILES: CCn1c(CO)nn(-c2cc3c(cc2F)OC(c2c(F)cccc2Cl)C(=O)N3C(C)C)c1=O]

Step A. 6-(3-(((tert-Butyldiphenylsilyl)oxy)methyl)-4-ethyl-5-oxo-4,5-dihydro-1H-1,2,4-triazol-1-yl)-2-(2-chloro-6-fluorophenyl)-7-fluoro-4-isopropyl-2H-benzo[b][1,4]oxazin-3(4H)-one. A mixture of 6-bromo-2-(2-chloro-6-fluoro-phenyl)-7-fluoro-4-isopropyl-1,4-benzoxazin-3-one (Intermediate 7, 40 mg, 96 μmol), 3-(((tert-butyldiphenylsilyl)oxy)methyl)-4-ethyl-1H-1,2,4-triazol-5(4H)-one (Intermediate 6, 73 mg, 190 μmol), Cs₂CO₃ (56 mg, 173 μmol), (1S,2S)—N1,N2-dimethylcyclohexane-1,2-diamine (11 mg, 77 μmol), KI (16 mg, 96 μmol) and CuI (18 mg, 96 μmol) in dioxane (2 mL) was degassed and purged with N₂ for 3 times, and then the mixture was stirred at 110° C. for 30 hrs under N₂ atmosphere. The reaction mixture was filtered and then washed with EtOAc (10 mL×3). The combined organic layers were concentrated under reduced pressure. The residue was purified by prep-TLC (SiO₂, petroleum ether/ethyl acetate=2/1) to give the title compound (40 mg, 45 μmol, 47% yield, 80% purity) as yellow oil. MS (ESI): mass calcd. for C₃₈H₃₉ClF₂N₄O₂Si, 716.2; m/z found, 717.2 [M+H]⁺. ¹H NMR (400 MHz, CDCl₃) δ=7.48-7.29 (m, 13H), 7.08-7.01 (m, 1H), 6.86 (m, J=10.0 Hz, 1H), 6.00 (s, 1H), 4.65 (s, 2H), 4.62-4.58 (m, 1H), 3.97-3.92 (m, 2H), 1.62-1.58 (m, 6H), 1.40 (t, J=7.2 Hz, 3H), 1.11 (m, 9H).

Step B. 2-(2-Chloro-6-fluorophenyl)-6-(4-ethyl-3-(hydroxymethyl)-5-oxo-4,5-dihydro-1H-1,2,4-triazol-1-yl)-7-fluoro-4-isopropyl-2H-benzo[b][1,4]oxazin-3(4H)-one. To a solution of 6-(3-(((tert-butyldiphenylsilyl)oxy)methyl)-4-ethyl-5-oxo-4,5-dihydro-1H-1,2,4-triazol-1-yl)-2-(2-chloro-6-fluorophenyl)-7-fluoro-4-isopropyl-2H-benzo[b][1,4]oxazin-3(4H)-one (40 mg, 56 μmol) in THF (1 mL) was added TBAF (1 M in THF, 84 mL,) at 0° C. The mixture was stirred at 15° C. for 5 hrs. The reaction mixture was diluted with H₂O (10 mL) and extracted with EtOAc (10 mL×3). The combined organic layers were washed with brine (10 mL×2), dried over anhydrous Na₂SO₄, filtered, and concentrated under reduced pressure. The residue was purified by prep-HPLC (condition A) to give the title compound (7.5 mg, 16 μmol, 28% yield, 100% purity) as white solid. MS (ESI): mass calcd. for C₂₂H₂₁ClF₂N₄O₄, 478.1; m/z found, 479.1 [M+H]⁺ ¹H NMR (400 MHz, CDCl3) δ=7.36-7.32 (m, 2H), 7.29 (s, 1H), 7.07-7.03 (m, 1H), 6.88 (d, J=10.4 Hz, 1H), 6.02 (s, 1H), 4.68 (s, 2H), 4.66-4.61 (m, 1H), 3.94-3.89 (m, 2H), 2.13 (s, 1H), 1.65-1.60 (m, 6H), 1.443 (t, J=7.2 Hz, 3H).

### Example 6: 1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one

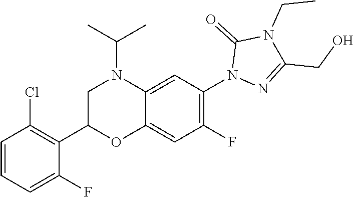 [SMILES: CCn1c(CO)nn(-c2cc3c(cc2F)OC(c2c(F)cccc2Cl)CN3C(C)C)c1=O]

Step A. 6-Bromo-2-(2-chloro-6-fluorophenyl)-7-fluoro-4-isopropyl-3,4-dihydro-2H-benzo[b][1,4]oxazine. To a solution of 6-bromo-2-(2-chloro-6-fluoro-phenyl)-7-fluoro-4-isopropyl-1,4-benzoxazin-3-one (Intermediate 7, 85 mg, 200 μmol) in THF (1 mL) was added BH₃·Me₂S (10 M, 82 μL) at 0° C. The mixture was stirred at 55° C. for 12 hrs. The reaction mixture was quenched by addition of MeOH (3 mL) then stirred at 15° C. for 1 hrs. The resulting mixture was concentrated under reduced pressure. The residue was purified by prep-TLC (SiO₂, Petroleum ether/Ethyl acetate=10/1) to give title compound (60 mg, 146 μmol, 72% yield, 98% purity) as colorless gum. MS (ESI): mass calcd. C₁₇H₁₅BrClF₂NO, 403.1; m/z found, 404.1 [M+1]⁺. ¹H NMR (400 MHz, CDCl₃) δ=7.30-7.28 (m, 1H), 7.26-7.25 (m, 1H), 7.08-7.03 (m, 1H), 6.90 (d, J=6.8 Hz, 1H), 6.69 (d, J=9.2 Hz, 1H), 5.64-5.62 (m, 1H), 4.03-3.97 (m, 1H), 3.40-3.29 (m, 2H), 1.26 (d, J=6.4 Hz, 3H), 1.15 (d, J=7.2 Hz, 3H).

Step B. 3-(((tert-Butyldiphenylsilyl)oxy)methyl)-1-(2-(2-chloro-6-fluorophenyl)-7-fluoro-4-isopropyl-3,4-dihydro-2H-benzo[b][1,4]oxazin-6-yl)-4-ethyl-1H-1,2,4-triazol-5(4H)-one. A mixture of 6-bromo-2-(2-chloro-6-fluoro-phenyl)-7-fluoro-4-isopropyl-2,3-dihydro-1,4-benzoxazine (73 mg, 181 μmol), 3-(((tert-butyldiphenylsilyl)oxy)methyl)-4-ethyl-1H-1,2,4-triazol-5(4H)-one (138 mg, 362 μmol), Cs₂CO₃ (106 mg, 326 μmol), (1S,2S)—N1,N2-dimethylcyclohexane-1,2-diamine (21 mg, 145 μmol), KI (30 mg, 181 μmol) and CuI (35 mg, 181 μmol) in dioxane (2 mL) was degassed and purged with N₂ for 3 times, and heated at 110° C. for 12 h under N₂ atmosphere. The reaction mixture was filtered and then washed with EtOAc (10 mL×3). The combined organic layers washed with brine (30 mL) and then separated. The combined organic layers were dried over anhydrous Na₂SO₄, filtered, and concentrated under reduced pressure. The residue was purified by prep-TLC (SiO₂, petroleum ether/ethyl acetate=3/1), TLC (petroleum ether/ethyl acetate=3/1) to give title compound (65 mg, 86 μmol, 47% yield, 92.9% purity) as brown oil. MS (ESI): mass calcd. for C₃₈H₄₁ClF₂N₄O₃Si, 702.3; m/z found, 703.4 [M+H]⁺. ¹H NMR (400 MHz, CDCl₃) δ=7.70-7.68 (m, 4H), 7.46-7.42 (m, 6H), 7.31-7.28 (m, 1H), 7.26-7.23 (m, 1H), 7.08-7.03 (m, 1H), 6.82 (d, J=7.2 Hz, 1H), 6.73 (J=10.8 Hz, 1H), 5.68-5.65 (m, 1H), 4.64 (s, 2H), 4.03-4.00 (m, 1H), 3.96-3.90 (m, 2H), 3.37-3.34 (m, 2H), 1.39 (t, J=7.2 Hz, 3H), 1.24 (d, J=6.4 Hz, 3H), 1.15 (d, J=6.8 Hz, 3H), 1.10 (s, 9H).

Step C. 1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one. To a solution of 3-(((tert-butyldiphenylsilyl)oxy)methyl)-1-(2-(2-chloro-6-fluorophenyl)-7-fluoro-4-isopropyl-3,4-dihydro-2H-benzo[b][1,4]oxazin-6-yl)-4-ethyl-1H-1,2,4-triazol-5(4H)-one (65 mg, 92.42 μmol) in THF (1 mL) was added TBAF (1 M in THF, 138.63 μL) at 0° C. The mixture was stirred at 15° C. for 2.5 hrs. The reaction mixture was diluted with H₂O (10 mL) and extracted with EtOAc (10 mL×3). The combined organic layers were washed with brine (10 mL×2), dried over anhydrous Na₂SO₄, filtered, and concentrated under reduced pressure. The residue was purified by prep-HPLC (condition A) to give title compound (23 mg, 50 μmol, 55% yield, 100% purity) as white solid. MS (ESI): mass calcd. C₂₂H₂₃ClF₂N₄O₃, 464.1; m/z found, 465.2 [M+H]⁺. ¹H NMR (400 MHz, CDCl₃) δ=7.32-7.28 (m, 1H), 7.26-7.24 (m, 1H), 7.09-7.03 (m, 1H), 6.86 (d, J=7.2 Hz, 1H), 6.75 (J=10.8 Hz, 1H), 5.69-5.66 (m, 1H), 4.66 (d, J=6.4 Hz, 2H), 4.06-4.01 (m, 1H), 3.93-3.87 (m, 2H), 3.38-3.35 (m, 2H), 2.03-2.00 (m, 1H), 1.42 (t, J=7.2 Hz, 3H), 1.24 (d, J=6.4 Hz, 3H), 1.15 (d, J=6.8 Hz, 3H).

### Example 7: (S*)-1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one

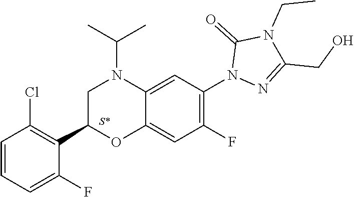 [SMILES: CCn1c(CO)nn(-c2cc3c(cc2F)O[C@@H](c2c(F)cccc2Cl)CN3C(C)C)c1=O]

1-(2-(2-chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one (Example 6, 550 mg, 1.18 mmol) was resolved by SFC to give peak 1, which was further purified by prep-HPLC (condition A) to afford the title compound (140 mg, retention time=1.18 min on SFC (Column: Chiralpak AS-3 50×4.6 mm I.D., 3 um; Mobile phase: Phase A for CO₂, and Phase B for EtOH (0.05% DEA); Gradient elution: EtOH (0.05% DEA) in CO₂ from 5% to 40%; Flow rate: 3 mL/min; Wavelength: 220 nm; Column Temp: 35° C.; Back Pressure: 100 Bar)). MS (ESI): mass calcd. C₂₂H₂₃ClF₂N₄O₃, 464.1; m/z found, 465.2 [M+H]⁺. ¹H NMR (400 MHz, CDCl₃) δ=7.32-7.28 (m, 1H), 7.27-7.24 (m, 1H) 7.08-7.05 (m, 1H), 6.88 (d, J=7.2 Hz, 1H), 6.76 (d, J=11.2 Hz, 1H), 5.70-5.67 (m, 1H), 4.67 (d, J=7.2 Hz, 2H), 4.06-4.02 (m, 1H), 3.94-3.88 (m, 2H), 3.39-3.36 (m, 2H), 2.04 (t, J=6.2 Hz, 1H), 1.43 (t, J=7.2 Hz, 3H), 1.26 (d, J=6.4 Hz, 3H), 1.16 (d, J=6.8 Hz, 3H).

### Example 8: (R*)-1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one

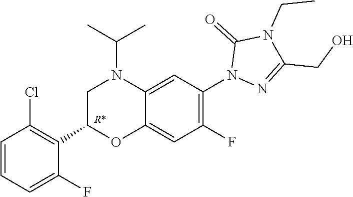 [SMILES: CCn1c(CO)nn(-c2cc3c(cc2F)O[C@H](c2c(F)cccc2Cl)CN3C(C)C)c1=O]

1-(2-(2-chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one (Example 6, 550 mg, 1.18 mmol) was resolved by SFC to give peak 2, which was further purified by prep-HPLC (condition A) to afford the title compound (86.9 mg, retention time=1.21 min on SFC (Column: Chiralpak AS-3 50×4.6 mm I.D., 3 um; Mobile phase: Phase A for CO₂, and Phase B for EtOH (0.05% DEA); Gradient elution: EtOH (0.05% DEA) in CO₂ from 5% to 40%; Flow rate: 3 mL/min; Wavelength: 220 nm; Column Temp: 35° C.; Back Pressure: 100 Bar)). MS (ESI): mass calcd. C₂₂H₂₃ClF₂N₄O₃, 464.1; m/z found, 465.2 [M+H]⁺. ¹H NMR (400 MHz, CDCl₃) δ=7.32-7.28 (m, 1H), 7.27-7.24 (m, 1H) 7.07-7.06 (m, 1H), 6.88 (d, J=6.8 Hz, 1H), 6.76 (d, J=10.8 Hz, 1H), 5.71-5.67 (m, 1H), 4.67 (d, J=7.2 Hz, 2H), 4.06-4.02 (m, 1H), 3.94-3.88 (m, 2H), 3.39-3.36 (m, 2H), 2.13 (t, J=6.2 Hz, 1H), 1.43 (t, J=7.2 Hz, 3H), 1.26 (d, J=6.4 Hz, 3H), 1.16 (d, J=6.8 Hz, 3H).

### Example 9: 7-(4-Ethyl-3-(hydroxymethyl)-5-oxo-4,5-dihydro-1H-1,2,4-triazol-1-yl)-6-fluoro-3-(2-fluoro-5-methylphenyl)-1-isopropylquinolin-4(1H)-one

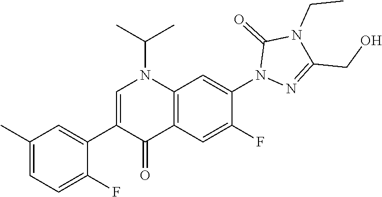 [SMILES: CCn1c(CO)nn(-c2cc3c(cc2F)c(=O)c(-c2cc(C)ccc2F)cn3C(C)C)c1=O]

The title compound was prepared in a manner analogous to Example 1, Step B to C, using (2-fluoro-5-methylphenyl)boronic acid instead of 3-fluorophenylboronic acid in Step B. MS (ESI): mass calcd. for C₂₄H₂₄F2N₄O₃, 454.48; m/z found, 455 [M+H]⁺. ¹H NMR (400 MHz, CDCl3) δ=8.29 (d, J=11.1 Hz, 1H), 8.02 (d, J=5.8 Hz, 1H), 7.96 (s, 1H), 7.45 (dd, J=7.2, 2.2 Hz, 1H), 7.16-7.09 (m, 1H), 7.05 (t, J=9.2 Hz, 1H), 4.90 (m, 1H), 4.58 (s, 2H), 4.09 (s, 1H), 3.89 (q, J=7.2 Hz, 2H), 2.37 (s, 3H), 1.59 (d, J=6.5 Hz, 6H), 1.37 (t, J=7.1 Hz, 3H).

### Example 10: 7-(4-Ethyl-3-(hydroxymethyl)-5-oxo-4,5-dihydro-TH-1,2,4-triazol-1-yl)-6-fluoro-1-isopropyl-3-(o-tolyl)quinolin-4(1H)-one

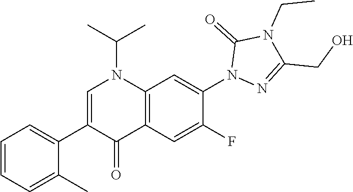 [SMILES: CCn1c(CO)nn(-c2cc3c(cc2F)c(=O)c(-c2ccccc2C)cn3C(C)C)c1=O]

The title compound was prepared in a manner analogous to Example 1, Step B to C, using o-tolylboronic acid instead of 3-fluorophenylboronic acid in Step B. MS (ESI): mass calcd. for C₂₄H₂₅FN₄O₃, 436.49; m/z found, 437 [M+H]⁺. ¹H NMR (400 MHz, CDCl3) δ=8.34 (d, J=11.1 Hz, 1H), 8.02 (d, J=5.9 Hz, 1H), 7.76 (s, 1H), 7.34-7.27 (m, 2H), 7.26-7.21 (m, 2H), 4.90 m, 1H), 4.66 (d, J=6.4 Hz, 2H), 3.93 (q, J=7.2 Hz, 2H), 2.72 (t, J=6.4 Hz, 1H), 2.27 (s, 3H), 1.59-1.57 (m, 6H), 1.43 (t, J=7.2 Hz, 3H).

### Example 11: 7-(4-Ethyl-3-(hydroxymethyl)-5-oxo-4,5-dihydro-1H-1,2,4-triazol-1-yl)-6-fluoro-1-isopropyl-3-(o-tolyl)-2,3-dihydroquinolin-4(1H)-one

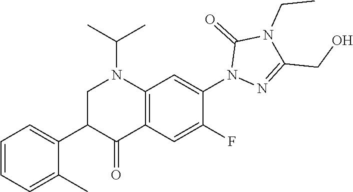 [SMILES: CCn1c(CO)nn(-c2cc3c(cc2F)C(=O)C(c2ccccc2C)CN3C(C)C)c1=O]

To a solution of 7-(4-ethyl-3-(hydroxymethyl)-5-oxo-4,5-dihydro-1H-1,2,4-triazol-1-yl)-6-fluoro-1-isopropyl-3-(o-tolyl)quinolin-4(1H)-one (34 mg, 0.078 mmol) in THF (2 mL), lithium aluminum hydride (LAH) (0.12 mL, 1.0 M, 0.12 mmol) was added slowly at −78° C. and stirred for 6 h. The reaction mixture was filtered and concentrated in vacuo. The residue was purified by prep-HPLC (Isco AcuuPrep, 30×100 mm, 20-100% ACN/water (10 mM NH₄OH), 15 min run time, Gemini C18 column) to provide the title compound (15 mg, 0.034 mmol, 44% yield) as a white solid. ¹H NMR (400 MHz, CDCl₃) δ=7.84 (dd, J=11.1, 1.1 Hz, 1H), 7.25-7.15 (m, 4H), 7.07 (d, J=6.5 Hz, 1H), 4.67 (s, 2H), 4.13 (m, 1H), 4.01 (dd, J=12.0, 4.9 Hz, 1H), 3.91 (q, J=7.2 Hz, 2H), 3.57 (dd, J=12.4, 5.0 Hz, 1H), 3.43 (t, J=12.2 Hz, 1H), 2.36 (s, 3H), 2.22 (s, 1H), 1.46-1.38 (m, 3H), 1.31 (d, J=6.5 Hz, 3H), 1.06 (d, J=6.5 Hz, 3H). MS (ESI): mass calcd. for C₂₄H₂₇FN₄O₃, 438.5; m/z found, 439.2 [M+H]+.

### Example 12: 3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropylquinolin-4(1H)-one

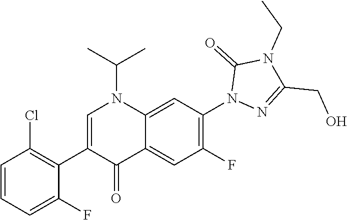 [SMILES: CCn1c(CO)nn(-c2cc3c(cc2F)c(=O)c(-c2c(F)cccc2Cl)cn3C(C)C)c1=O]

Step A. 7-Bromo-3-(2-chloro-6-fluorophenyl)-6-fluoro-1-isopropylquinolin-4(1H)-one. To a solution of 7-bromo-3-(2-chloro-6-fluorophenyl)-6-fluoroquinolin-4(1H)-one (Intermediate 2, 740 mg, 2 mmol, in anhydrous DMF (20 mL) was added 2-iodopropane (1 g, 6 mmol) and Cs₂CO₃ (2 g, 6 mmol). The reaction mixture was heated at 80° C. for 1 h. The mixture was cooled to room temperature, diluted with DCM (50 mL), and washed with water (50 mL). The organic layer was separated, and the aqueous layer was extracted with ethyl acetate (100 mL). The organic layers were combined, dried over anhydrous Na₂SO₄, filtered, and concentrated. Purification (FCC, SiO₂, 20-60% EtOAc in heptane) afforded the title compound as a white solid (84 mg, 10% yield). LCMS (ESI): mass calcd. for C₁₈H₁₃BrClF₂NO, 411.0; m/z found, 411.9 [M+H]⁺. ¹H NMR (400 MHz, CDCl₃) δ8.27 (d, J=8.80 Hz, 1H), 7.87 (d, J=5.38 Hz, 1H), 7.79 (s, 1H), 7.27-7.36 (m, 2H), 7.04-7.15 (m, 1H), 4.86 (m, 1H), 1.59-1.63 (m, 6H) ppm.

Step B. 7-(3-((Benzyloxy)methyl)-4-ethyl-5-oxo-4,5-dihydro-1H-1,2,4-triazol-1-yl)-3-(2-chloro-6-fluorophenyl)-6-fluoro-1-isopropylquinolin-4(1H)-one. To a mixture of 7-bromo-3-(2-chloro-6-fluorophenyl)-6-fluoro-1-isopropylquinolin-4(1H)-one (84 mg, 0.2 mmol), 3-((benzyloxy)methyl)-4-ethyl-1H-1,2,4-triazol-5(4H)-one (142 mg, 0.6 mmol), CuI (39 mg, 0.2 mmol), trans-N,N′-dimethylcyclohexane-1,2-diamine (29 mg, 0.2 mmol), KI (34 mg, 0.2 mmol), and Cs₂CO₃ (199 mg, 0.2 mmol) in a microwave tube (20 mL) was added anhydrous 1,4-dioxane (6 mL). The reaction mixture was heated under microwave irradiation at 120° C. for 2 h. The mixture was cooled to room temperature and filtered through a pad of silica gel. The silica gel was washed with ethyl acetate. The combined filtrate was concentrated. Purification (FCC, SiO₂, 50-100% EtOAc in heptane) afforded the title compound as a white solid (22 mg, 19%). LCMS (ESI): mass calcd. for C₃₀H₂₇ClF₂N₄O₃, 564.2; m/z found, 565.0 [M+H]⁺. ¹H NMR (400 MHz, CDCl₃) δ8.38 (d, J=11.25 Hz, 1H), 8.04 (d, J=5.87 Hz, 1H), 7.83 (s, 1H), 7.28-7.46 (m, 7H), 7.04-7.18 (m, 1H), 4.90 (td, J=6.48, 13.45 Hz, 1H), 4.64 (s, 2H), 4.55 (s, 2H), 3.89 (q, J=7.05 Hz, 2H), 1.59-1.63 (m, 6H), 1.39 (t, J=7.05 Hz, 3H) ppm.

Step C. 3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropylquinolin-4(1H)-one. To a solution of 7-(3-((benzyloxy)methyl)-4-ethyl-5-oxo-4,5-dihydro-1H-1,2,4-triazol-1-yl)-3-(2-chloro-6-fluorophenyl)-6-fluoro-1-isopropylquinolin-4(1H)-one (22 mg, 0.04 mmol) in DCM (1 mL) at −78° C. was added a toluene solution (1 M) of BCl₃ (0.12 mL, 0.12 mmol) under nitrogen. The reaction mixture was stirred at −78° C. for 1 h. MeOH (2 mL) was at −78° C. and then the mixture was stirred for 0.5 h. The mixture was diluted with DCM, washed with saturated aqueous NaHCO₃ solution. The organic layer was separated, dried with Na₂SO₄, and concentrated. The residue was purified by preparative reversed phase HPLC (Stationary phase: Boston Prime C18, 5 μm, 150×25 mm; Mobile phase: H₂O (0.04% NH₃·H₂O+10 mM NH₄HCO₃) (A) -MeCN (B), gradient elution: 40-70% B in A over 8 min, flow rate: 25 mL/min) to give the title compound as a white solid (12.6 mg, yield: 68%). LCMS (ESI): mass calcd. for C₂₃H₂₁ClF₂N₄O₃, 474.1; m/z found, 475.0 [M+H]⁺. ¹H NMR (400 MHz, CDCl₃) δ8.36 (d, J=11.25 Hz, 1H), 8.06 (d, J=5.87 Hz, 1H), 7.83 (s, 1H), 7.27-7.38 (m, 2H), 7.07-7.17 (m, 1H), 4.90 (td, J=6.60, 13.21 Hz, 1H), 4.70 (d, J=6.36 Hz, 2H), 3.94 (q, J=7.23 Hz, 2H), 2.44 (br t, J=6.36 Hz, 1H), 1.56-1.67 (m, 6H), 1.44 (t, J=7.23 Hz, 3H) ppm.

### Example 13: Racemic 4-Ethyl-2-(7-fluoro-4-isopropyl-2-(o-tolyl)-1,2,3,4-tetrahydroquinolin-6-yl)-5-(hydroxymethyl)-2,4-dihydro-3H-1,2,4-triazol-3-one

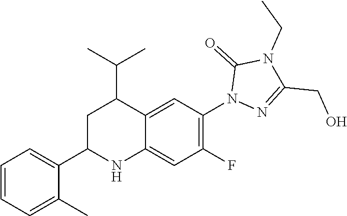 [SMILES: CCn1c(CO)nn(-c2cc3c(cc2F)NC(c2ccccc2C)CC3C(C)C)c1=O]

Step A. N-(4-Bromo-3-fluorophenyl)-4-methyl-3-oxopentanamide. A mixture of methyl 4-methyl-3-oxopentanoate (10 g, 69.36 mmol), 4-bromo-3-fluoroaniline (14.5 g, 76.31 mmol), and Et₃N (1.8 g, 17.79 mmol) in toluene (70 mL) was heated to 70° C. The reaction mixture was stirred at 70° C. for 1 hour and then gradually heated to 110° C. The reaction mixture was stirred at 110° C. overnight. After cooling to room temperature, the mixture was washed with 5% aq. HCl (100 mL) and water (100 mL×2). The organic layer was dried with anhydrous Na₂SO₄, filtered, and concentrated under reduced pressure. Purification (FCC, SiO₂, petroleum ether/ethyl acetate=1/0 to 9/1) afforded the title compound (7.8 g, 21.29 mmol, 30.70% yield) as a brown solid. MS (ESI): mass calcd. for C₁₂H₁₃BrFNO₂, 301.0; m/z found, 303.8 [M+H]⁺. ¹H NMR (400 MHz, CDCl₃) δ=9.51 (br s, 1H), 7.67 (dd, J=2.3, 10.5 Hz, 1H), 7.48 (t, J=8.2 Hz, 1H), 7.13 (dd, J=1.5, 8.8 Hz, 1H), 3.63 (s, 2H), 2.75 (td, J=7.0, 13.9 Hz, 1H), 1.20 (d, J=6.8 Hz, 6H).

Step B. 6-Bromo-7-fluoro-4-isopropylquinolin-2-ol. A solution of N-(4-bromo-3-fluorophenyl)-4-methyl-3-oxopentanamide (3.7 g, 10.05 mmol) in conc. H₂SO₄ (19 mL) was stirred at 50° C. for 2 days. The reaction mixture was cooled to room temperature and poured onto a mixture of ice and sat. aq. Na₂CO₃ (700 mL). The reaction mixture was filtered, and the filter cake was washed with H₂O (100 mL×2). Purification of the crude solid (FCC, SiO₂, petroleum ether/ethyl acetate=1/0 to 7/3) afforded the title compound (1.75 g, 6.14 mmol, 61.05% yield) as a yellow solid. MS (ESI): mass calcd. for C₁₂H₁₁BrFNO, 283.0; m/z found, 285.8 [M+H]⁺. ¹H NMR (400 MHz, CDCl₃) δ=11.99 (br s, 1H), 7.97 (d, J=7.0 Hz, 1H), 7.20 (d, J=8.8 Hz, 1H), 6.61 (s, 1H), 3.33 (td, J=6.9, 13.6 Hz, 1H), 1.36 (d, J=6.8 Hz, 6H); ¹⁹F NMR (376 MHz, CDCl₃) δ=−103.06 (s, 1F).

Step C. 3-((Benzyloxy)methyl)-4-ethyl-1-(7-fluoro-2-hydroxy-4-isopropylquinolin-6-yl)-1H-1,2,4-triazol-5(4H)-one. 3-((Benzyloxy)methyl)-4-ethyl-1H-1,2,4-triazol-5(4H)-one (Intermediate 3, step B) (295 mg, 1.27 mmol) and Cs₂CO₃ (618 mg, 1.90 mmol) was added slowly into the solution of 6-bromo-7-fluoro-4-isopropylquinolin-2-ol (300 mg, 1.05 mmol) in dioxane (8 mL) at room temperature under N₂. To the resulting reaction mixture was added CuI (100 mg, 525.1 μmol), KI (123 mg, 741 μmol) and trans-N,N-dimethylcyclohexane-1,2-diamine (90 mg, 632.7 μmol) under N₂. Upon completion of the addition, the reaction mixture was stirred at 115° C. for 16 hours. The reaction mixture was diluted with H₂O (20 mL) and extracted with ethyl acetate (20 mL×3). The organic layers were combined, dried over Na₂SO₄, filtered, and evaporated under reduced pressure. Purification (FCC, SiO₂, 0-80% ethyl acetate in petroleum ether) afforded the title compound (360 mg, 719.8 μmol, 68.28% yield) as a white solid. MS (ESI): mass calcd. for C₂₄H₂₅FN₄O₃, 436.2; m/z found, 437.2 [M+H]⁺. ¹H NMR (400 MHz, CDCl₃) δ=7.93 (d, J=7.5 Hz, 1H), 7.37-7.32 (m, 5H), 7.17 (br d, J=10.4 Hz, 1H), 6.59 (s, 1H), 4.60 (s, 2H), 4.50 (s, 2H), 3.85 (m, J=7.4 Hz, 2H), 3.36-3.28 (m, 1H), 1.37-1.31 (m, 9H); ¹⁹F NMR (376 MHz, CDCl₃) δ=−116.37-−116.46 (m, 1F).

Step D. 3-((Benzyloxy)methyl)-1-(2-chloro-7-fluoro-4-isopropylquinolin-6-yl)-4-ethyl-1H-1,2,4-triazol-5(4H)-one. POCl₃ (3 mL) was added slowly into a solution of 3-((benzyloxy)methyl)-4-ethyl-1-(7-fluoro-2-hydroxy-4-isopropylquinolin-6-yl)-1H-1,2,4-triazol-5(4H)-one (360 mg, 719.8 μmol) in toluene (5 mL) at room temperature. The reaction mixture was stirred at 95° C. for 1 hour. The reaction mixture was slowly added to H₂O (20 mL) and extracted with DCM (20 mL×3). The organic layers were combined, dried over Na₂SO₄, filtered, and evaporated under reduced pressure. Purification (FCC, SiO₂, 0-80% ethyl acetate in petroleum ether) afforded the title compound (280 mg, 408 μmol, 57% yield) as a white solid. MS (ESI): mass calcd. for C₂₄H₂₄ClFN₄O₂, 454.2; m/z found, 455.1 [M+H]⁺. ¹H NMR (400 MHz, CDCl₃) δ=8.31 (d, J=8.0 Hz, 1H), 7.84 (d, J=11.0 Hz, 1H), 7.45-7.33 (m, 5H), 7.31 (s, 1H), 4.65 (s, 2H), 4.60-4.54 (m, 2H), 3.91 (m, J=7.1 Hz, 2H), 3.67 (td, J=6.8, 13.7 Hz, 1H), 1.45-1.40 (m, 9H); ¹⁹F NMR (376 MHz, CDCl₃) δ=−116.37 (s, 1F).

Step E. 3-((Benzyloxy)methyl)-4-ethyl-1-(7-fluoro-4-isopropyl-2-(o-tolyl)quinolin-6-yl)-1H-1,2,4-triazol-5(4H)-one. 2-Methylphenylboronic acid (428 mg, 3.15 mmol), K₂CO₃ (871 mg, 6.30 mmol), and Pd-118 (137 mg, 210 μmol) were added to a solution of 3-((benzyloxy)methyl)-1-(2-chloro-7-fluoro-4-isopropylquinolin-6-yl)-4-ethyl-TH-1,2,4-triazol-5(4H)-one (Intermediate 2, 1 g, 2.20 mmol) in a mixture of dioxane/H₂O (v/v, 5/1, 15 mL) at room temperature under N₂. The reaction mixture was stirred at 50° C. for 2 hours under N₂. The reaction mixture was diluted with H₂O (50 mL) and extracted with ethyl acetate (60 mL×3). The combined organic layers were dried with anhydrous Na₂SO₄, filtered, and concentrated under reduced pressure. Purification (FCC, SiO₂, gradient elution: 0-50% ethyl acetate in petroleum ether) afforded the title compound (930 mg, 1.82 mmol, 83% yield) as a white solid. MS (ESI): mass calcd. for C₃₁H₃₁FN₄O₂, 510.2; m/z found, 511.2 [M+H]⁺. ¹H NMR (400 MHz, CDCl₃) δ=8.31 (d, J=7.9 Hz, 1H), 7.92 (d, J=11.5 Hz, 1H), 7.52-7.47 (m, 1H), 7.43 (s, 1H), 7.39-7.29 (m, 8H), 4.62 (s, 2H), 4.54 (s, 2H), 3.89 (m, J=7.3 Hz, 2H), 3.78-3.66 (m, 1H), 2.39 (s, 3H), 1.42-1.36 (m, 9H); ¹⁹F NMR (376 MHz, CDCl₃) δ=−114.77-125.23 (m, 1F).

Step F. 3-((Benzyloxy)methyl)-4-ethyl-1-(7-fluoro-4-isopropyl-2-(o-tolyl)-1,2,3,4-tetrahydroquinolin-6-yl)-1H-1,2,4-triazol-5(4H)-one. NaBH₃CN (492 mg, 7.83 mmol) was added gradually to a mixture of 3-((benzyloxy) methyl)-4-ethyl-1-(7-fluoro-4-isopropyl-2-(o-tolyl)quinolin-6-yl)-1H-1,2,4-triazol-5(4H)-one (500 mg, 979 μmol) in AcOH (10 mL) at room temperature. The reaction mixture was stirred at room temperature overnight. The reaction mixture was diluted H₂O (20 mL) and extracted with ethyl acetate (30 mL×3). The combined organic layers were dried with anhydrous Na₂SO₄, filtered, and concentrated under reduced pressure. Purification (FCC, SiO₂, gradient elution: 0-50% ethyl acetate in petroleum ether) afforded the title compound (400 mg, 777 μmol, 79% yield, 100% purity) as white solid. MS (ESI): mass calcd. for C₃₁H₃₅FN₄O₂, 514.3; m/z found, 515.3 [M+H]⁺. ¹H NMR (400 MHz, CDCl₃) δ=7.54 (d, J=7.3 Hz, 1H), 7.42-7.33 (m, 5H), 7.29 (s, 1H), 7.24-7.17 (m, 3H), 6.34 (d, J=11.5 Hz, 1H), 4.68-4.63 (m, 1H), 4.60 (s, 2H), 4.51 (s, 2H), 3.85 (q, J=7.1 Hz, 2H), 3.02 (br d, J=11.5 Hz, 1H), 2.46 (qd, J=6.9, 10.5 Hz, 1H), 2.38 (s, 3H), 2.01 (ddd, J=2.8, 5.5, 13.1 Hz, 1H), 1.65 (br d, J=12.0 Hz, 1H), 1.36 (t, J=7.2 Hz, 3H), 1.04 (d, J=7.0 Hz, 3H), 0.77 (d, J=6.8 Hz, 3H); ¹⁹F NMR (376 MHz, CDCl₃) δ=−125.05 (br s, 1F).

Step G. Racemic 4-Ethyl-2-(7-fluoro-4-isopropyl-2-(o-tolyl)-1,2,3,4-tetrahydroquinolin-6-yl)-5-(hydroxymethyl)-2,4-dihydro-3H-1,2,4-triazol-3-one. BCl₃ (1 M solution in toluene, 0.78 mL, 0.78 mmol) was added to a stirred solution of 3-((benzyloxy)methyl)-4-ethyl-1-(7-fluoro-4-isopropyl-2-(o-tolyl)-1,2,3,4-tetrahydroquinolin-6-yl)-1H-1,2,4-triazol-5(4H)-one (80 mg, 155 μmol) in DCM (4 mL) at −78° C. The reaction mixture was stirred at −78° C. for 1 hour. The reaction mixture was quenched with MeOH (1.5 mL) at −78° C. and stirred at −78° C. for 0.5 hour. The reaction mixture was diluted with DCM (15 mL) and washed with sat. aq. NaHCO₃ (18 mL). The organic phase was dried with anhydrous Na₂SO₄, filtered, and concentrated under reduced pressure. Purification (preparative reversed phase HPLC, Stationary phase: Boston Prime C18, 5 μm, 150×30 mm; Mobile phase: water (0.05% NH₃H₂O+10 mM NH₄HCO₃) (A) -MeCN (B), gradient elution: 55-85% B in A over 7 min, flow rate: 25 mL/min) afforded the title compound (35 mg, 81.48 μmol, 52% yield, 98% purity) as white powder. MS (ESI): mass calcd. for C₂₄H₂₉FN₄O₂, 424.2; m/z found, 425.1 [M+H]⁺. ¹H NMR (400 MHz, CDCl₃) δ=7.46 (d, J=7.5 Hz, 1H), 7.21 (s, 1H), 7.15-7.09 (m, 3H), 6.26 (d, J=11.8 Hz, 1H), 4.61-4.55 (m, 3H), 3.99 (s, 1H), 3.81 (q, J=7.2 Hz, 2H), 2.99-2.89 (m, 1H), 2.43-2.34 (m, 1H), 2.31 (s, 3H), 2.05 (t, J=6.4 Hz, 1H), 1.97-1.89 (m, 1H), 1.62-1.52 (m, 1H), 1.33 (t, J=7.2 Hz, 3H), 0.97 (d, J=6.9 Hz, 3H), 0.69 (d, J=6.8 Hz, 3H); ¹⁹F NMR (376 MHz, CDCl₃) δ=−124.97-125.38 (m, 1F).

### Example 14: Racemic 4-Ethyl-2-(6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one

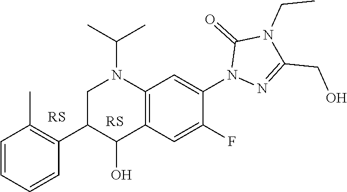 [SMILES: CCn1c(CO)nn(-c2cc3c(cc2F)C(O)C(c2ccccc2C)CN3C(C)C)c1=O]

Step A. 7-(3-((Benzyloxy)methyl)-4-ethyl-5-oxo-4,5-dihydro-1H-1,2,4-triazol-1-yl)-6-fluoro-1-isopropyl-3-(o-tolyl)quinolin-4(1H)-one. The title compound was prepared in a manner analogous to Example 1, Step B, using o-tolylboronic acid instead of 3-fluorophenylboronic acid. LCMS (ES-API): mass calcd. for C₃₁H₃₁FN₄O₃, 526.2; m/z found, 527.1 [M+H]⁺. ¹H NMR (400 MHz, CDCl₃) δ8.38 (d, J=11.1 Hz, 1H), 8.01 (d, J=5.8 Hz, 1H), 7.75 (s, 1H), 7.43-7.32 (m, 5H), 7.30 (dd, J=4.2, 2.1 Hz, 2H), 7.26-7.23 (m, 2H), 4.89 (h, J=6.6 Hz, 1H), 4.64 (s, 2H), 4.55 (s, 2H), 3.89 (q, J=7.2 Hz, 2H), 2.27 (s, 3H), 1.58 (d, J=6.6 Hz, 6H), 1.39 (t, J=7.2 Hz, 3H) ppm.

Step B. Racemic 7-(3-((Benzyloxy)methyl)-4-ethyl-5-oxo-4,5-dihydro-1H-1,2,4-triazol-1-yl)-6-fluoro-1-isopropyl-3-(o-tolyl)-2,3-dihydroquinolin-4(1H)-one. The title compound was prepared in a manner analogous to Example 11 except substituting 7-(3-((benzyloxy)methyl)-4-ethyl-5-oxo-4,5-dihydro-1H-1,2,4-triazol-1-yl)-6-fluoro-1-isopropyl-3-(o-tolyl)quinolin-4(1H)-one for 7-(4-ethyl-3-(hydroxymethyl)-5-oxo-4,5-dihydro-1H-1,2,4-triazol-1-yl)-6-fluoro-1-isopropyl-3-(o-tolyl)quinolin-4(1H)-one. LCMS (ES-API): mass calcd. for C₃₁H₃₃FN₄O₃, 528.2; m/z found, 529.2 [M+H]⁺. ¹H NMR (400 MHz, CDCl₃) δ7.84 (d, J=10.76 Hz, 1H), 7.29-7.43 (m, 5H), 7.13-7.26 (m, 4H), 7.04-7.09 (m, 1H), 4.61 (s, 2H), 4.53 (s, 2H), 4.06-4.21 (m, 1H), 4.01 (dd, J=4.89, 11.74 Hz, 1H), 3.86 (q, J=7.01 Hz, 2H), 3.53-3.62 (m, 1H), 3.35-3.49 (m, 1H), 2.36 (s, 3H), 1.36 (t, J=7.09 Hz, 3H), 1.31 (d, J=6.36 Hz, 3H), 1.06 (d, J=6.85 Hz, 3H) ppm.

Step C. Racemic 5-((Benzyloxy)methyl)-4-ethyl-2-(6-fluoro-4-hydroxy-1-isopropyl-3-(o-tolyl)-1,2,3,4-tetrahydroquinolin-7-yl)-2,4-dihydro-3H-1,2,4-triazol-3-one. To a solution of racemic 7-(3-((benzyloxy)methyl)-4-ethyl-5-oxo-4,5-dihydro-1H-1,2,4-triazol-1-yl)-6-fluoro-1-isopropyl-3-(o-tolyl)-2,3-dihydroquinolin-4(1H)-one (143 mg, 0.27 mmol) in methanol (10 mL) was added anhydrous CeCl₃ (133 mg, 0.54 mmol) and NaBH₄ (102 mg, 2.7 mmol) respectively. The reaction mixture was stirred at room temperature for 1 h. The reaction mixture was partitioned between ethyl acetate and water. The organic layer was separated, dried over MgSO₄, filtered, and concentrated. Purification (FCC, SiO₂, 40 g; 30-50% ethyl acetate in heptane) afforded the title compound as a white foam: 140 mg, yield 98%. LCMS (ES-API): mass calcd. for C₃₁H₃₅FN₄O₃, 530.3; m/z found, 531.2 [M+H]⁺. ¹H NMR (400 MHz, CDCl₃) δ7.31-7.45 (m, 6H), 7.16-7.25 (m, 3H), 7.09 (d, J=10.27 Hz, 1H), 6.91 (d, J=5.87 Hz, 1H), 4.65-4.73 (m, 1H), 4.60 (s, 2H), 4.52 (s, 2H), 4.05-4.19 (m, 1H), 3.85 (q, J=7.17 Hz, 2H), 3.53-3.64 (m, 1H), 3.31 (td, J=3.12, 11.86 Hz, 1H), 3.22 (dd, J=2.69, 11.00 Hz, 1H), 2.35 (s, 3H), 1.69 (d, J=3.42 Hz, 1H), 1.36 (t, J=7.34 Hz, 3H), 1.22-1.26 (m, 3H), 1.18 (d, J=6.85 Hz, 3H) ppm.

Step D. Racemic 4-Ethyl-2-(6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one. To a solution of racemic 5-((benzyloxy)methyl)-4-ethyl-2-(6-fluoro-4-hydroxy-1-isopropyl-3-(o-tolyl)-1,2,3,4-tetrahydroquinolin-7-yl)-2,4-dihydro-3H-1,2,4-triazol-3-one (140 mg, 0.26 mmol) in DCM (10 mL) at −78° C. was added a toluene solution (1 M) of BCl₃ (0.79 mL, 0.79 mmol) under nitrogen. The reaction mixture was stirred at −78° C. for 1 h. MeOH (2 mL) was at −78° C. and then the reaction mixture was stirred for 0.5 h. The reaction mixture was diluted with DCM, washed with saturated aqueous NaHCO₃ solution. The organic layer was separated, dried with Na₂SO₄, and concentrated under reduced pressure. Purification (preparative reversed phase HPLC, Gemini C18 110A, 5 uM 100×30 mm, 10-90% CH₃CN in water with 10 mM NH₄OH, 60 mL/min) afforded the title compound as a white solid (72 mg, yield 62%). LCMS (ES-API): mass calcd. for C₂₄H₂₉FN₄O₃, 440.2; m/z found, 441.3 [M+H]⁺. ¹H NMR (400 MHz, CDCl₃) δ7.35 (br d, J=6.85 Hz, 1H), 7.22 (br s, 3H), 7.08 (d, J=10.27 Hz, 1H), 6.90 (d, J=6.36 Hz, 1H), 4.68 (br s, 1H), 4.62 (br d, J=5.87 Hz, 2H), 4.04-4.20 (m, 1H), 3.88 (q, J=6.85 Hz, 2H), 3.54-3.64 (m, 1H), 3.18-3.35 (m, 2H), 2.44 (br t, J=6.11 Hz, 1H), 2.35 (s, 3H), 1.84 (br d, J=3.42 Hz, 1H), 1.40 (t, J=7.09 Hz, 3H), 1.24 (br d, J=6.36 Hz, 3H), 1.18 (d, J=6.85 Hz, 3H) ppm.

### Example 15: 4-Ethyl-2-((3S*,4S*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one

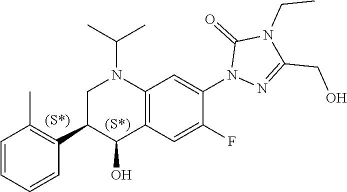 [SMILES: CCn1c(CO)nn(-c2cc3c(cc2F)[C@@H](O)[C@@H](c2ccccc2C)CN3C(C)C)c1=O]

SFC chiral separation [Stationary phase: DAICEL CHIRALPAK AD-H, 5 μm 250 mm×30 mm); Mobile phase: Supercritical CO₂ (A) -EtOH (0.1% NH₃·H₂O IPA) (B), gradient elution: 35% B in A at 60 mL/min] of racemic 4-ethyl-2-(6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one (Example 14, 50 mg, 0.11 mmol) afforded the title compound as a yellow powder (1.3 mg, 2.3%); mass calcd. for C₂₄H₂₉FN₄O₃, 440.2; m/z found, 441.1[M+H]⁺; ¹H NMR (400 MHz, DMSO-d₆) δ7.25 (d, J=11.2 Hz, 1H), 7.21-7.15 (m, 3H), 7.13-7.09 (m, 1H), 6.72 (d, J=6.2 Hz, 1H), 5.74 (br d, J=12.8 Hz, 1H), 5.38 (br d, J=7.5 Hz, 1H), 5.32 (t, J=4.8 Hz, 1H), 4.76 (br s, 1H), 4.44 (s, 2H), 3.98 (quin, J=6.6 Hz, 1H), 3.76 (q, J=7.2 Hz, 2H), 3.21-3.03 (m, 2H), 2.34 (s, 3H), 1.29-1.26 (m, 3H), 1.13 (d, J=6.4 Hz, 3H), 0.99 (d, J=6.6 Hz, 3H); ¹⁹F NMR (376 MHz, DMSO-d₆) δ −137.99 (s, 1F) ppm.

### Example 16: 4-Ethyl-2-((3R*,4R*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one

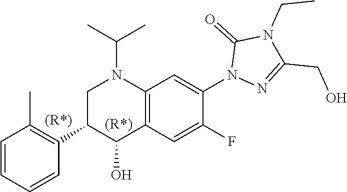 [SMILES: CCn1c(CO)nn(-c2cc3c(cc2F)[C@H](O)[C@H](c2ccccc2C)CN3C(C)C)c1=O]

SFC chiral separation [Stationary phase: DAICEL CHIRALPAK AD-H, 5 μm 250 mm×30 mm); Mobile phase: Supercritical CO₂ (A) -EtOH (0.1% NH₃·H₂O IPA) (B), gradient elution: 35% B in A at 60 mL/min] of racemic 4-ethyl-2-(6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one (Example 14, 50 mg, 0.11 mmol) afforded the title compound as a yellow powder (1.2 mg, 2.2%); mass calcd. for C₂₄H₂₉FN₄O₃, 440.2; m/z found, 441.0 [M+H]⁺. ¹H NMR (400 MHz, DMSO-d₆) δ7.25 (d, J=11.1 Hz, 1H), 7.21-7.14 (m, 3H), 7.14-7.08 (m, 1H), 6.72 (d, J=6.3 Hz, 1H), 5.73 (br d, J=15.7 Hz, 1H), 5.38 (br s, 1H), 5.32 (t, J=5.1 Hz, 1H), 4.77 (br s, 1H), 4.44 (s, 2H), 4.03-3.92 (m, 1H), 3.76 (q, J=7.1 Hz, 2H), 3.20-3.08 (m, 2H), 2.34 (s, 3H), 1.29-1.26 (m, 3H), 1.13 (d, J=6.6 Hz, 3H), 0.99 (d, J=6.6 Hz, 3H); ¹⁹F NMR (376 MHz, DMSO-d₆) δ −137.99 (s, 1F) ppm.

### Example 17: 4-Ethyl-2-((3S*,4R*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one

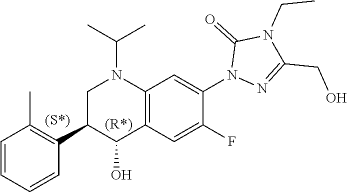 [SMILES: CCn1c(CO)nn(-c2cc3c(cc2F)[C@H](O)[C@@H](c2ccccc2C)CN3C(C)C)c1=O]

SFC chiral separation [Stationary phase: DAICEL CHIRALPAK AD-H, 5 μm 250 mm×30 mm); Mobile phase: Supercritical CO₂ (A) -EtOH (0.1% NH₃·H₂O IPA) (B), gradient elution: 35% B in A at 60 mL/min] of racemic 4-ethyl-2-(6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one (Example 14, 50 mg, 0.11 mmol) afforded the title compound as a white powder (14.3 mg, 28%); mass calcd. for C₂₄H₂₉FN₄O₃, 440.2; m/z found, 441.1 [M+H]⁺. ¹H NMR (400 MHz, DMSO-d₆) δ7.41-7.31 (m, 1H), 7.21-7.10 (m, 3H), 7.08 (d, J=10.4 Hz, 1H), 6.78 (d, J=6.3 Hz, 1H), 5.73 (t, J=5.8 Hz, 1H), 5.24 (d, J=5.1 Hz, 1H), 4.57 (br d, J=2.0 Hz, 1H), 4.45 (d, J=5.7 Hz, 2H), 4.13-4.01 (m, 1H), 3.76 (q, J=7.1 Hz, 2H), 3.62-3.44 (m, 1H), 3.20-3.02 (m, 2H), 2.29 (s, 3H), 1.27 (t, J=7.2 Hz, 3H), 1.18 (d, J=6.4 Hz, 3H), 1.11 (d, J=6.4 Hz, 3H); ¹⁹F NMR (376 MHz, DMSO-d₆) δ −139.04 (s, 1F) ppm.

### Example 18: 4-Ethyl-2-((3R*,4S*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one

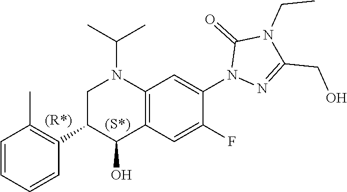 [SMILES: CCn1c(CO)nn(-c2cc3c(cc2F)[C@@H](O)[C@H](c2ccccc2C)CN3C(C)C)c1=O]

SFC chiral separation [Stationary phase: DAICEL CHIRALPAK AD-H, 5 μm 250 mm×30 mm); Mobile phase: Supercritical CO₂ (A) -EtOH (0.1% NH₃·H₂O IPA) (B), gradient elution: 35% B in A at 60 mL/min] of racemic 4-ethyl-2-(6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one (Example 14, 50 mg, 0.11 mmol) afforded the title compound as a white powder (13.9 mg, 27%); purity; mass calcd. for C₂₄H₂₉FN₄O₃, 440.2; m/z found, 441.2 [M+H]⁺. ¹H NMR (400 MHz, DMSO-d₆) δ7.40-7.31 (m, 1H), 7.20-7.11 (m, 3H), 7.08 (d, J=10.4 Hz, 1H), 6.78 (d, J=6.3 Hz, 1H), 5.73 (br s, 1H), 5.24 (br d, J=3.9 Hz, 1H), 4.57 (br s, 1H), 4.45 (s, 2H), 4.07 (td, J=6.4, 13.1 Hz, 1H), 3.76 (q, J=7.1 Hz, 2H), 3.59-3.47 (m, 1H), 3.17-3.07 (m, 2H), 2.29 (s, 3H), 1.27 (t, J=7.2 Hz, 3H), 1.18 (d, J=6.6 Hz, 3H), 1.11 (d, J=6.4 Hz, 3H); ¹⁹F NMR (376 MHz, DMSO-d₆) δ −139.04 (s, 1F) ppm.

### Example 19: Racemic 7-(4-Ethyl-3-(hydroxymethyl)-5-oxo-4,5-dihydro-1H-1,2,4-triazol-1-yl)-6-fluoro-1-isopropyl-3-methyl-3-(o-tolyl)-23-dihydroquinolin-4(1H)-one

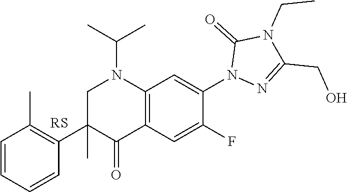 [SMILES: CCn1c(CO)nn(-c2cc3c(cc2F)C(=O)C(C)(c2ccccc2C)CN3C(C)C)c1=O]

Step A. Racemic 7-(3-((Benzyloxy)methyl)-4-ethyl-5-oxo-4,5-dihydro-1H-1,2,4-triazol-1-yl)-6-fluoro-1-isopropyl-3-methyl-3-(o-tolyl)-2,3-dihydroquinolin-4(1H)-one. To a solution of racemic 7-(3-((benzyloxy)methyl)-4-ethyl-5-oxo-4,5-dihydro-1H-1,2,4-triazol-1-yl)-6-fluoro-1-isopropyl-3-(o-tolyl)-2,3-dihydroquinolin-4(1H)-one (Example 14, Step B., 80 mg, 0.15 mmol) in anhydrous THF (5 mL) at −78° C. was added a THF solution (2 M) of LDA (0.15 mL, 0.3 mmol). The reaction mixture was stirred at −78° C. for 0.5 h, then iodomethane (0.047 mL, 0.76 mmol) was added. The reaction mixture was slowly warmed to 0° C. and stirred for 1 h. The reaction mixture was quenched by the addition of aqueous HCl (1 M, 10 mL). The reaction mixture was extracted with ethyl acetate (2×30 mL). The organic layers were separated, combined, dried over MgSO₄, filtered, and concentrated. Purification (FCC, SiO₂, 40 g; 50% ethyl acetate in heptane) afforded the title compound as a white foam: 55 mg, yield 67%. LCMS (ES-API): mass calcd. for C₃₂H₃₅FN₄O₃, 542.3; m/z found, 543.2 [M+H]⁺. ¹H NMR (400 MHz, CDCl₃) δ 7.88 (d, J=10.76 Hz, 1H), 7.31-7.46 (m, 6H), 7.17-7.25 (m, 3H), 7.10 (d, J=5.87 Hz, 1H), 4.61 (s, 2H), 4.52 (s, 2H), 4.02-4.21 (m, 1H), 3.80-3.91 (m, 2H), 3.63 (d, J=12.72 Hz, 1H), 3.04 (d, J=12.72 Hz, 1H), 2.27 (s, 3H), 1.62 (s, 3H), 1.36 (t, J=7.09 Hz, 3H), 1.25-1.29 (m, 3H), 1.13 (d, J=6.36 Hz, 3H) ppm.

Step B. Racemic 7-(4-Ethyl-3-(hydroxymethyl)-5-oxo-4,5-dihydro-TH-1,2,4-triazol-1-yl)-6-fluoro-1-isopropyl-3-methyl-3-(o-tolyl)-2,3-dihydroquinolin-4(1H)-one. To a solution of racemic 7-(3-((benzyloxy)methyl)-4-ethyl-5-oxo-4,5-dihydro-TH-1,2,4-triazol-1-yl)-6-fluoro-1-isopropyl-3-methyl-3-(o-tolyl)-2,3-dihydroquinolin-4(1H)-one (55 mg, 0.10 mmol) in DCM (10 mL) at −78° C. was added a toluene solution (1 M) of BCl₃ (0.3 mL, 0.3 mmol) under nitrogen. The reaction mixture was stirred at −78° C. for 1 h. MeOH (2 mL) was added the reaction mixture at −78° C., and then the reaction mixture was stirred for 0.5 h. The reaction mixture was diluted with DCM, washed with saturated aqueous NaHCO₃ solution. The organic layer was separated, dried with Na₂SO₄, and concentrated under reduced pressure. Purification (FCC, SiO₂, 40 g, 50-80% EtOAc in heptane) afforded the title compound, which was further purified by preparative reversed phase HPLC (Gemini C18 110A, 5 uM 100×30 mm, 10-90% CH₃CN in water with 10 mM NH₄OH, 60 mL/min) to afford the title compound as a white solid (15 mg, yield 33%). LCMS (ES-API): mass calcd. for C₂₅H₂₉FN₄O₃, 452.2; m/z found, 453.2 [M+H]⁺; ¹H NMR (400 MHz, CDCl₃) δ7.88 (d, J=10.76 Hz, 1H), 7.37-7.46 (m, 1H), 7.19-7.26 (m, 3H), 7.11 (d, J=5.87 Hz, 1H), 4.68 (d, J=6.36 Hz, 2H), 4.03-4.22 (m, 1H), 3.91 (q, J=7.17 Hz, 2H), 3.63 (d, J=12.72 Hz, 1H), 3.04 (d, J=12.72 Hz, 1H), 2.27 (s, 3H), 2.08 (br t, J=6.36 Hz, 1H), 1.63 (s, 3H), 1.42 (t, J=7.09 Hz, 3H), 1.28 (d, J=6.36 Hz, 3H), 1.13 (d, J=6.85 Hz, 3H) ppm.

### Example 20: Racemic 4-Ethyl-2-(6-fluoro-4-hydroxy-1-isopropyl-4-methyl-3-(o-tolyl)-1,2,3,4-tetrahydroquinolin-7-yl)-5-(hydroxymethyl)-2,4-dihydro-3H-1,2,4-triazol-3-one

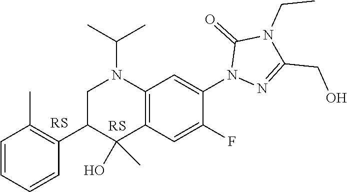 [SMILES: CCn1c(CO)nn(-c2cc3c(cc2F)C(C)(O)C(c2ccccc2C)CN3C(C)C)c1=O]

Step A. Racemic 5-((Benzyloxy)methyl)-4-ethyl-2-(6-fluoro-4-hydroxy-1-isopropyl-4-methyl-3-(o-tolyl)-1,2,3,4-tetrahydroquinolin-7-yl)-2,4-dihydro-3H-1,2,4-triazol-3-one. To a solution of racemic 7-(3-((benzyloxy)methyl)-4-ethyl-5-oxo-4,5-dihydro-1H-1,2,4-triazol-1-yl)-6-fluoro-1-isopropyl-3-(o-tolyl)-2,3-dihydroquinolin-4(1H)-one (Example 14, Step B., 94 mg, 0.18 mmol) in anhydrous THF (12 mL) at −78° C. was added a THF solution (3 M) of methylmagnesium bromide (0.18 mL, 0.54 mmol). The reaction mixture was then warmed to 0° C. and stirred for 2 h. The reaction mixture was quenched by the addition of aqueous saturated NH₄Cl. The reaction mixture was extracted with ethyl acetate (2×). The organic layers were combined, dried over MgSO₄, filtered, and concentrated under reduced pressure. Purification (FCC, SiO₂, 40 g; 50% ethyl acetate in heptane) afforded the title compound as a light-yellow foam (87 mg, yield 89%). LCMS (ES-API): mass calcd. for C₃₂H₃₇FN₄O₃, 544.3; m/z found, 545.2 [M+H]⁺. ¹H NMR (400 MHz, CDCl₃) δ7.29-7.43 (m, 7H), 7.11-7.24 (m, 3H), 6.84 (d, J=6.36 Hz, 1H), 4.61 (s, 2H), 4.52 (s, 2H), 4.02-4.17 (m, 1H), 3.86 (q, J=7.34 Hz, 2H), 3.29-3.48 (m, 3H), 2.37 (s, 3H), 1.75 (s, 1H), 1.50 (s, 3H), 1.36 (t, J=7.34 Hz, 3H), 1.20 (d, J=6.85 Hz, 3H), 1.10 (d, J=6.36 Hz, 3H) ppm.

Step B. Racemic 4-Ethyl-2-(6-fluoro-4-hydroxy-1-isopropyl-4-methyl-3-(o-tolyl)-1,2,3,4-tetrahydroquinolin-7-yl)-5-(hydroxymethyl)-2,4-dihydro-3H-1,2,4-triazol-3-one. To a solution of racemic 5-((benzyloxy)methyl)-4-ethyl-2-(6-fluoro-4-hydroxy-1-isopropyl-4-methyl-3-(o-tolyl)-1,2,3,4-tetrahydroquinolin-7-yl)-2,4-dihydro-3H-1,2,4-triazol-3-one (87 mg, 0.16 mmol) in DCM (20 mL) at −78° C. was added a toluene solution (1 M) of BCl₃ (0.48 mL, 0.48 mmol) under nitrogen. The reaction mixture was stirred at −78° C. for 1 h. MeOH (2 mL) was added to the reaction mixture at −78° C. and then the mixture was stirred for 0.5 h. The reaction mixture was diluted with DCM, washed with saturated aqueous NaHCO₃ solution. The organic layer was separated, dried with Na₂SO₄, and concentrated under reduced pressure. Purification (preparative reversed phase HPLC, Gemini C18 110A, 5 μM 100×30 mm, 10-90% CH₃CN in water with 10 mM NH₄OH, 60 mL/min) afforded the title compound as a white solid (7 mg, yield 10%); LCMS (ES-API): mass calcd. for C₂₅H₃₁FN₄O₃, 454.2; m/z found, 455.1 [M+H]⁺; ¹H NMR (400 MHz, CDCl₃) δ7.38 (br d, J=7.34 Hz, 1H), 7.32 (br d, J=11.25 Hz, 1H), 7.08-7.24 (m, 3H), 6.84 (br d, J=5.87 Hz, 1H), 4.56-4.68 (m, 2H), 4.01-4.15 (m, 1H), 3.89 (q, J=7.17 Hz, 2H), 3.25-3.51 (m, 3H), 2.37 (s, 3H), 2.27 (s, 1H), 1.83 (s, 1H), 1.50 (s, 3H), 1.41 (br t, J=7.34 Hz, 3H), 1.20 (br d, J=6.36 Hz, 3H), 1.10 (br d, J=6.36 Hz, 3H) ppm; and 4-ethyl-2-(6-fluoro-1-isopropyl-4-methyl-3-(o-tolyl)-1,2-dihydroquinolin-7-yl)-5-(hydroxymethyl)-2,4-dihydro-3H-1,2,4-triazol-3-one (Example 21, 20 mg, yield: 29%).

### Example 21: 4-Ethyl-2-(6-fluoro-1-isopropyl-4-methyl-3-(o-tolyl)-1,2-dihydroquinolin-7-yl)-5-(hydroxymethyl)-2,4-dihydro-3H-1,2,4-triazol-3-one

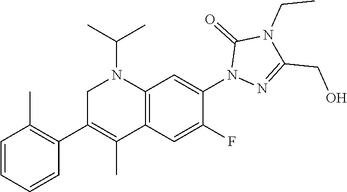 [SMILES: CCn1c(CO)nn(-c2cc3c(cc2F)C(C)=C(c2ccccc2C)CN3C(C)C)c1=O]

The title compound was obtained as the second product in Example 20 Step B: a white powder (20 mg, yield: 29%). LCMS (ES-API): mass calcd. for C₂₅H₂₉FN₄O₂, 436.2; m/z found, 437.1 [M+H]⁺; ¹H NMR (400 MHz, CDCl₃) δ7.44 (d, J=11.74 Hz, 1H), 7.07-7.25 (m, 3H), 7.02 (d, J=11.25 Hz, 1H), 6.74 (d, J=6.36 Hz, 1H), 4.67 (d, J=5.87 Hz, 2H), 3.78-4.09 (m, 5H), 2.28 (s, 3H), 2.08 (br t, J=6.36 Hz, 1H), 1.73 (s, 3H), 1.42 (t, J=7.09 Hz, 3H), 1.20 (d, J=6.85 Hz, 3H), 1.18 (d, J=6.36 Hz, 3H) ppm.

### Example 22: 4-Ethyl-2-((2R*,4S*)-7-fluoro-4-isopropyl-2-(0-tolyl)-1,2,3,4-tetrahydroquinolin-6-yl)-5-(hydroxymethyl)-2,4-dihydro-3H-1,2,4-triazol-3-one

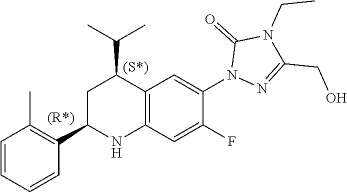 [SMILES: CCn1c(CO)nn(-c2cc3c(cc2F)N[C@@H](c2ccccc2C)C[C@H]3C(C)C)c1=O]

SFC chiral separation of Racemic 4-ethyl-2-(7-fluoro-4-isopropyl-2-(o-tolyl)-1,2,3,4-tetrahydroquinolin-6-yl)-5-(hydroxymethyl)-2,4-dihydro-3H-1,2,4-triazol-3-one (Example 13, 120 mg, 282 μmol) (Column: DAICEL CHIRALCEL OD-H 250×30 mm I.D., 5 μm; mobile phase: CO₂ (A) -Methanol (0.05% DEA); isocratic: 30% B in A; flow rate: 60 mL/min; column temp.: 40° C.; ABPR: 100 bar.) afforded the title compound (35 mg, 82.17 μmol, 29% yield) as white powder. MS (ESI): mass calcd. for C₂₄H₂₉FN₄O₂, 424.2; m/z found, 425.2 [M+H]⁺. ¹H NMR (400 MHz, CDCl₃) δ=7.45 (d, J=7.4 Hz, 1H), 7.21-7.09 (m, 4H), 6.25 (d, J=11.7 Hz, 1H), 4.57 (dd, J=2.4, 11.2 Hz, 1H), 4.52 (s, 2H), 4.02 (s, 1H), 3.79 (q, J=7.2 Hz, 2H), 2.96-2.88 (m, 1H), 2.56 (br s, 1H), 2.37 (qd, J=6.9, 10.5 Hz, 1H), 2.30 (s, 3H), 1.97-1.89 (m, 1H), 1.58-1.49 (m, 1H), 1.31 (t, J=7.2 Hz, 3H), 0.96 (d, J=6.9 Hz, 3H), 0.68 (d, J=6.8 Hz, 3H); ¹⁹F NMR (376 MHz, CDCl₃) δ=−125.16 (s, 1F).

### Example 23: 4-Ethyl-2-((2R*,4S*)-7-fluoro-4-isopropyl-2-(o-tolyl)-1,2,3,4-tetrahydroquinolin-6-yl)-5-(hydroxymethyl)-2,4-dihydro-3H-1,2,4-triazol-3-one

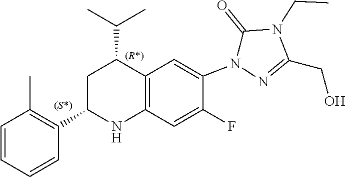 [SMILES: CCn1c(CO)nn(-c2cc3c(cc2F)N[C@H](c2ccccc2C)C[C@@H]3C(C)C)c1=O]

SFC chiral separation of Racemic 4-ethyl-2-(7-fluoro-4-isopropyl-2-(o-tolyl)-1,2,3,4-tetrahydroquinolin-6-yl)-5-(hydroxymethyl)-2,4-dihydro-3H-1,2,4-triazol-3-one (Example 13, 120 mg, 282 μmol) (Column: DAICEL CHIRALCEL OD-H 250×30 mm I.D., 5 μm; mobile phase: CO₂ (A) -Methanol (0.05% DEA); isocratic: 30% B in A; flow rate: 60 mL/min; column temp.: 40° C.; ABPR: 100 bar.) afforded the title compound as a white powder (35 mg, 82.1 μmol, 29% yield). MS (ESI): mass calcd. for C₂₄H₂₉FN₄O₂, 424.2; m/z found, 425.2 [M+H]⁺. ¹H NMR (400 MHz, CDCl₃) δ=7.45 (d, J=7.4 Hz, 1H), 7.21-7.09 (m, 4H), 6.25 (d, J=11.7 Hz, 1H), 4.57 (dd, J=2.4, 11.2 Hz, 1H), 4.52 (s, 2H), 4.02 (s, 1H), 3.79 (q, J=7.2 Hz, 2H), 2.96-2.88 (m, 1H), 2.56 (br s, 1H), 2.37 (qd, J=6.9, 10.5 Hz, 1H), 2.30 (s, 3H), 1.97-1.89 (m, 1H), 1.58-1.49 (m, 1H), 1.31 (t, J=7.2 Hz, 3H), 0.96 (d, J=6.9 Hz, 3H), 0.68 (d, J=6.8 Hz, 3H); ¹⁹F NMR (376 MHz, CDCl₃) δ=−125.16 (s, 1F).

### Example 24: 1-(1-Acetyl-7-fluoro-4-isopropyl-2-(o-tolyl)-1,2,3,4-tetrahydroquinolin-6-yl)-4-ethyl-3-(hydroxymethyl)-1H-1,2,4-triazol-5(4H)-one

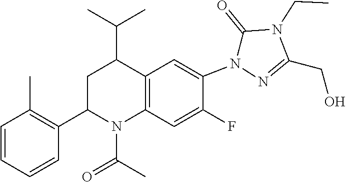 [SMILES: CCn1c(CO)nn(-c2cc3c(cc2F)N(C(C)=O)C(c2ccccc2C)CC3C(C)C)c1=O]

Step A. 1-(1-Acetyl-7-fluoro-4-isopropyl-2-(o-tolyl)-1,2,3,4-tetrahydroquinolin-6-yl)-3-((benzyloxy)methyl)-4-ethyl-1H-1,2,4-triazol-5(4H)-one. To a mixture of 3-((benzyloxy)methyl)-4-ethyl-1-(7-fluoro-4-isopropyl-2-(o-tolyl)-1,2,3,4-tetrahydroquinolin-6-yl)-1H-1,2,4-triazol-5(4H)-one (Example 13, product from Step F, 75 mg, 141 μmol) and AcCl (13.4 mg, 170 μmol) in DCM (1 mL) was added pyridine (33.7 mg, 425 μmol) at room temperature. The reaction mixture was stirred at room temperature overnight. The reaction mixture was diluted with H₂O (12 mL) and extracted with ethyl acetate (10 mL×3). The combined organic layers were dried over Na₂SO₄, filtered, and concentrated under reduced pressure. Purification (FCC, SiO₂, 0-70% ethyl acetate in petroleum ether) afforded the title compound (65 mg, 116 μmol, 82% yield) as white solid. MS (ESI): mass calcd. for C₃₃H₃₇FN₄O₃, 556.3; m/z found, 557.2 [M+H]⁺. ¹H NMR (400 MHz, CDCl₃) δ=7.45 (d, J=7.9 Hz, 1H), 7.43-7.32 (m, 6H), 7.16-7.08 (m, 3H), 6.91 (s, 1H), 5.62 (s, 1H), 4.63 (s, 2H), 4.53 (s, 2H), 3.89 (q, J=7.2 Hz, 2H), 2.54 (s, 3H), 2.53-2.40 (m, 4H), 2.16 (s, 3H), 1.39 (t, J=7.2 Hz, 3H), 1.16 (d, J=6.6 Hz, 3H), 0.93 (d, J=6.7 Hz, 3H); ¹⁹F NMR (376 MHz, CDCl₃) δ=−121.91 (br s, 1F).

Step B. 1-(1-Acetyl-7-fluoro-4-isopropyl-2-(o-tolyl)-1,2,3,4-tetrahydroquinolin-6-yl)-4-ethyl-3-(hydroxymethyl)-1H-1,2,4-triazol-5(4H)-one. BCl₃ (1 M solution in toluene, 0.90 mL, 0.90 mmol) was added to 1-(1-acetyl-7-fluoro-4-isopropyl-2-(o-tolyl)-1,2,3,4-tetrahydroquinolin-6-yl)-3-((benzyloxy)methyl)-4-ethyl-1H-1,2,4-triazol-5(4H)-one (100 mg, 179 μmol) in DCM (5 mL) at −78° C. under N₂. The reaction mixture was stirred at −78° C. for 1 hour. The reaction mixture was quenched with MeOH (2.5 mL) at −78° C., stirred at −78° C. for 0.5 hour. The reaction mixture was diluted with DCM (20 mL), washed with sat. aq. NaHCO₃ (18 mL). The organic phase was dried with anhydrous Na₂SO₄, filtered, and concentrated under reduced pressure. Purification (preparative reverse phase HPLC, Stationary phase: Boston Prime C18, 5 μm, 150×30 mm; Mobile phase: water (0.05% NH₃H₂O+10 mM NH₄HCO₃) (A) -MeCN (B), gradient elution: 50-80% B in A over 7 min, flow rate: 25 mL/min) afforded the title compound (58 mg, 124 μmol, 69% yield, 100% purity) as an off-white powder. MS (ESI): mass calcd. for C₂₆H₃₁FN₄O₃, 466.2; m/z found, 467.2 [M+H]⁺. ¹H NMR (400 MHz, CDCl₃) δ=7.45 (d, J=8.0 Hz, 1H), 7.27-7.19 (m, 1H), 7.16-7.05 (m, 3H), 6.91 (s, 1H), 5.60 (s, 1H), 4.68 (d, J=6.2 Hz, 2H), 3.93 (q, J=7.2 Hz, 2H), 2.54 (s, 3H), 2.53-2.40 (m, 3H), 2.21 (t, J=6.4 Hz, 1H), 2.15 (s, 3H), 1.44 (t, J=7.2 Hz, 3H), 1.16 (d, J=6.7 Hz, 3H), 0.92 (d, J=6.7 Hz, 3H); ¹⁹F NMR (376 MHz, CDCl₃) δ=−118.55-−127.35 (m, 1F).

### Biological Data

DHODH inhibitory activities of the compounds of Examples 1-24 were assessed using the following assays. The half maximal effective concentration values (IC₅₀) are summarized in Table 2.

### Biological Assays

### In Vitro Assay: DHODH Enzymatic Assay

To detect DHODH enzyme activities, dichloroindophenol (DCIP) is added as the final electron acceptor in the assay. DCIP can accept electrons from the reduced coenzyme Q generated in the assay, or from dihydroorotate (DHO) via FMN by binding presumably to the ubiquinone pocket. DCIP solutions are blue, with an intense absorbance around 600 nm, but becomes colorless upon reduction (J. Biol. Chem. (1986) 261, 11386). The assay buffer contained 50 nM HEPES, pH 7.5, 150 mM NaCl, 0.5 mM EDTA, and 0.1% Triton X-100 in MilliQ water. Substrate consisting of 20 mM DHO, 5 mM CoQ₆, and 1 mM DCIP in assay buffer, initiates the reaction. The assay is run in end-point mode by quenching the reaction with the potent DHODH inhibitor brequinar. Absorbance measurements were obtained using the BMG Phera Star plate-reading spectrophotomer. Purified human DHODH was purchased from Proteros (cat. No. PR-0044). Chemicals were purchased from Sigma-Aldrich, Teknova, and Avanti Polar Lipids. Liquid handling was performed using Labcyte Echo and Formulatrix Tempest.

### In Vitro Assay: MOLM-13 Cellular Assay

MOLM-13 cells were obtained from DSMZ and were maintained in RPMI 1640+Glutamax+25 mM HEPES (Invitrogen, catalog number 72400) supplemented with 10% heat inactivated fetal bovine serum (FBS; Invitrogen, catalog number 16140). The day prior to assay set-up, cells were pelleted, resuspended in fresh media, counted, and cells were plated at 0.4×10⁶ cell/mL in a T150 flask. On the day of the assay, cells were pelleted, resuspend in fresh media, counted, and seeded at 5,000 cells/well in white opaque 96-well tissue culture treated microplates (Perkin Elmer, catalog number 6005680). Cells were exposed to different concentrations of test compounds at 37° C., 5% CO₂ for 72 hours immediately after seeding. Cell viability was acquired on a Perkin Elmer Envision 2104 multilabel reader using the CellTiter-Glo assay (Promega) according to the manufacturer's instructions.

TABLE 2

| | DHODH Enzymatic | MOLM-13 Cellular |
|---|---|---|
| Example # | Assay IC₅₀ (nM) | Assay IC₅₀ (nM) |
| 1 | 528 | 400 |
| 2 | 588 | 100 |
| 3 | 293 | 100 |
| 4 | 2.42 | 3 |
| 5 | 42.3 | 50 |
| 6 | 0.504 | ~0.3 |
| 7 | 0.256 | 0.1 |
| 8 | 5.5 | 3 |
| 9 | 58.2 | >100 |
| 10 | 7.59 | 9 |
| 11 | 0.303 | 0.5 |
| 12 | 20 | 30 |
| 13 | 51.7 | 20 |
| 14 | 5.62 | 4 |
| 15 | 7.9 | 4 |
| 16 | 2.24 | 2 |
| 17 | 173 | ~100 |
| 18 | 6.91 | 3 |
| 19 | 7.06 | 4 |
| 20 | 0.607 | 0.5 |
| 21 | 3.71 | 2 |
| 22 | 113 | 30 |
| 23 | 49.2 | 10 |
| 24 | 5530 | >100 |

### Enumerated Embodiments

Exemplary numbered embodiments of the invention are shown below.
1. A compound having the structure of Formula (I):

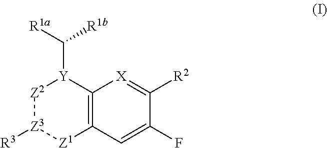

wherein
X is CH or, optionally, N;
Y is CH or N;
Z¹ is selected from the group consisting of: CH₂, C(CH₃), CH(OH), C(CH₃)(OH), O, C═O, and NR^{a};
   R^{a} is selected from the group consisting of: H, CH₂(C═O)NH₂, (C═O)CH₃, and (C═O)NHCH₃;
Z² is CH, CH₂, or C═O;
Z³ is C, CH or C(CH₃);
each [symbol] is independently a single bond or a double bond;
   wherein
   when Z³ is CH or C(CH₃), [symbol] between Z² and Z³ is a single bond, and [symbol] between Z³ and Z¹ is a single bond;
   when Z³ is C, Z² is CH, [symbol] between Z² and Z³ is a double bond, and [symbol] between Z³ and Z¹ is a single bond;
   or
   when Z¹ is C(CH₃), [symbol] between Z² and Z³ is a single bond, and [symbol] between Z³ and Z¹ is a double bond;
R^{1a} is selected from the group consisting of: C₁₋₆alkyl; C₁₋₆alkyl substituted with OH, or OCH₃; C₁₋₆haloalkyl; C₁₋₆haloalkyl substituted with OH, or OCH₃; and C₃₋₆cycloalkyl;
R^{1b} is CH₃ or CHF₂; or R^{1a} and R^{1b} come together to form C₃₋₆cycloalkyl; C₃₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl;
R² is

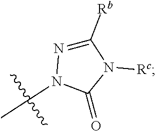

wherein
   R^{b} is C₁₋₆alkyl substituted with a member selected from the group consisting of OH, halo, CN, OC₁₋₆alkyl, OC₁₋₆haloalkyl and OC₃₋₆cycloalkyl; and
   R^{c} is selected from the group consisting of C₁₋₆alkyl, C₁₋₆haloalkyl, and C₃₋₆cycloalkyl;
R³ is

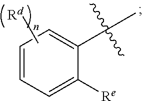

wherein
   R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of OH, and OCH₃; N(CH₃)₂; OH; CN and OC₁₋₆alkyl;
   R^{e} is selected from the group consisting of H, halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of OH, and OCH₃; OH; OC₁₋₆alkyl; and C₃₋₆cycloalkyl;
   n is 1, or 2; and
   R⁴ is H or CH₃;
   or a pharmaceutically acceptable salt, solvate, stereoisomer, tautomer, isotopic variant, or N-oxide thereof.
2. The compound according to embodiment 1, wherein X is CH; or a pharmaceutically acceptable salt, solvate, stereoisomer, tautomer, isotopic variant, or N-oxide thereof.
3. The compound according to any of embodiments 1-2, wherein Y is CH; or a pharmaceutically acceptable salt, solvate, stereoisomer, tautomer, isotopic variant, or N-oxide thereof.
4. The compound according to any of embodiments 1-2, wherein Y is N; or a pharmaceutically acceptable salt, solvate, stereoisomer, tautomer, isotopic variant, or N-oxide thereof.
5. The compound according to any of embodiments 1-4, wherein

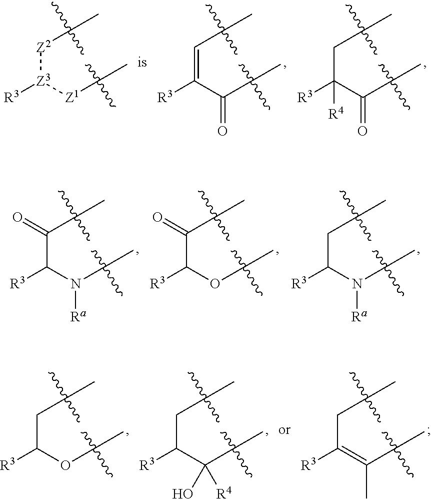

and R⁴ is H or CH₃; or a pharmaceutically acceptable salt, solvate, stereoisomer, tautomer, isotopic variant, or N-oxide thereof.
6. The compound according to any of embodiments 1-4, wherein

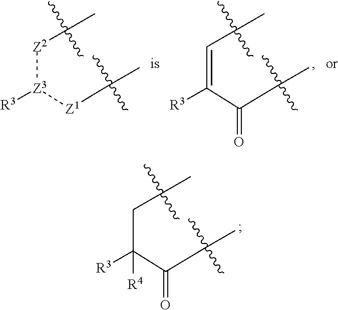

or a pharmaceutically acceptable salt, solvate, stereoisomer, tautomer, isotopic variant, or N-oxide thereof
7. The compound according to any of embodiments 1-4, wherein

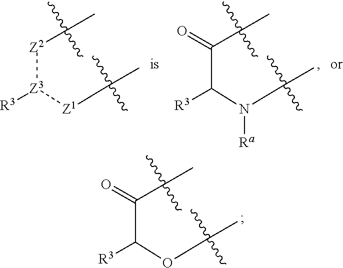

or a pharmaceutically acceptable salt, solvate, stereoisomer, tautomer, isotopic variant, or N-oxide thereof.
8. The compound according to any of embodiments 1-4, wherein

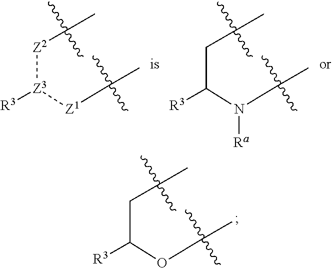

or a pharmaceutically acceptable salt, solvate, stereoisomer, tautomer, isotopic variant, or N-oxide thereof.
9. The compound according to any of embodiments 1-4, wherein

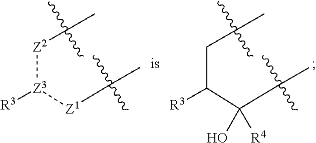

or a pharmaceutically acceptable salt, solvate, stereoisomer, tautomer, isotopic variant, or N-oxide thereof.
10. The compound according to any of embodiments 1-4, wherein

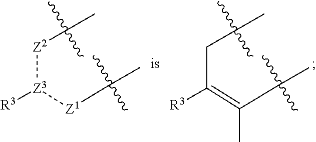

or a pharmaceutically acceptable salt, solvate, stereoisomer, tautomer, isotopic variant, or N-oxide thereof.
11. The compound according to any of embodiments 1-10, wherein R^{a} is C₁₋₄alkyl substituted with OH; CH₂(C═O)NH₂, (C═O)CH₃, and (C═O)NHCH₃; or a pharmaceutically acceptable salt, solvate, stereoisomer, tautomer, isotopic variant, or N-oxide thereof.
12. The compound according to any of embodiments 1-11, wherein R^{1a} is C₁₋₄alkyl; C₁₋₄alkyl substituted with OH, or OCH₃; C₁₋₄haloalkyl; C₁₋₄haloalkyl substituted with OH, or OCH₃; or C₃₋₆cycloalkyl; or a pharmaceutically acceptable salt, solvate, stereoisomer, tautomer, isotopic variant, or N-oxide thereof.
13. The compound according to any of embodiments 1-11, wherein R^{1a} is CH₃ or CF₃; or a pharmaceutically acceptable salt, solvate, stereoisomer, tautomer, isotopic variant, or N-oxide thereof.
14. The compound according to any of embodiments 1-13, wherein R^{1b} is CH₃ or CHF₂; or a pharmaceutically acceptable salt, solvate, stereoisomer, tautomer, isotopic variant, or N-oxide thereof.
15. The compound according to any of embodiments 1-13, wherein R^{1b} is CH₃; or a pharmaceutically acceptable salt, solvate, stereoisomer, tautomer, isotopic variant, or N-oxide thereof.
16. The compound according to any of embodiments 1-11, wherein R^{1a} and R^{1b} come together to form cyclopropyl, cyclobutyl, cyclopentyl, or cyclohexyl; cyclopropyl, cyclobutyl, cyclopentyl, or cyclohexyl each independently substituted with one, two, three or four members selected from the group consisting of: halo, OH, C₁₋₄alkyl, and C₁₋₄haloalkyl; oxetanyl; tetrahydrofuranyl; and tetrahydropyranyl; or a pharmaceutically acceptable salt, solvate, stereoisomer, tautomer, isotopic variant, or N-oxide thereof.
17. The compound according to any of embodiments 1-16, wherein R² is

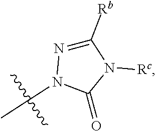

where
R^{b} is C₁₋₄alkyl substituted with OH, halo, CN, OC₁₋₄alkyl, OC₁₋₄haloalkyl or OC₃₋₆cycloalkyl; and
R^{c} is C₁₋₄alkyl, C₁₋₄haloalkyl, or C₃₋₆cycloalkyl; or a pharmaceutically acceptable salt, solvate, stereoisomer, tautomer, isotopic variant, or N-oxide thereof.
18. The compound according to any of embodiments 1-16, wherein R² is

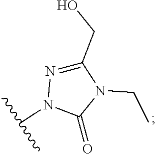 [SMILES: *n1nc(CO)n(CC)c1=O]

or a pharmaceutically acceptable salt, solvate, stereoisomer, tautomer, isotopic variant, or N-oxide thereof.
19. The compound according to any of embodiments 1-18, wherein R³ is

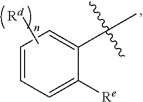

where
R^{d} is H; halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; CN; or OC₁₋₄alkyl;
R^{e} is H, halo; C₁₋₄alkyl; C₁₋₄alkyl substituted with OH, OCH₃, SCH₃, or OCF₃; C₁₋₄haloalkyl; or C₁₋₄haloalkyl substituted with OH, or OCH₃; and
n is 1 or 2;
or a pharmaceutically acceptable salt, solvate, stereoisomer, tautomer, isotopic variant, or N-oxide thereof.
20. The compound according to embodiment 19, wherein R^{e} is H, SCH₃, Cl, F, or CH₃; or a pharmaceutically acceptable salt, solvate, stereoisomer, tautomer, isotopic variant, or N-oxide thereof.
21. The compound according to embodiment 1, having the structure of Formula (IA):

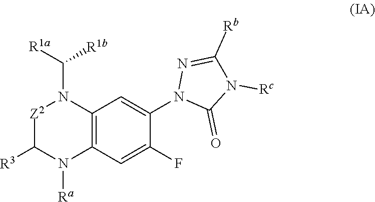

wherein
Z² is CH₂ or C═O;
R^{1a} is C₁₋₄alkyl;
R^{1b} is C₁₋₄alkyl or C₁₋₄haloalkyl;
R^{a} is H, C₁₋₆alkyl substituted with OH; CH₂(C═O)NH₂, (C═O)CH₃, and (C═O)NHCH₃;
R^{b} is C₁₋₄alkyl substituted with a member selected from the group consisting of: OH, halo, CN, OC₁₋₄alkyl, OC₁₋₄haloalkyl and OC₃₋₆cycloalkyl;
R^{c} is selected from the group consisting of: C₁₋₄alkyl, C₁₋₄haloalkyl, and C₃₋₆cycloalkyl; and
R³ is

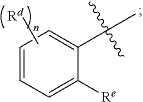

R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl; and
n is 1, or 2;
   or a pharmaceutically acceptable salt, solvate, stereoisomer, tautomer, isotopic variant, or N-oxide thereof.
22. The compound according to embodiment 1, having the structure of Formula (IB):

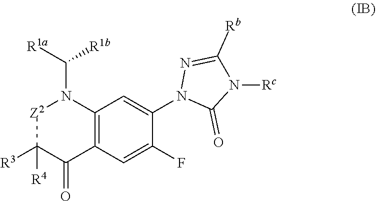

wherein
when Z² is CH, [symbol] is a double bond and R⁴ is absent; when Z² is CH₂, [symbol] is a single bond and R⁴ is H or CH₃;
R^{1a} is C₁₋₄alkyl;
R^{1b} is C₁₋₄alkyl or C₁₋₄haloalkyl;
R^{b} is C₁₋₄alkyl substituted with OH, halo, CN, OC₁₋₄alkyl, OC₁₋₄haloalkyl or OC₃₋₆cycloalkyl;
R^{c} is C₁₋₄alkyl, C₁₋₄haloalkyl, or C₃₋₆cycloalkyl; and
R³ is

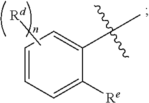

wherein
   R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
   R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of OH, and OCH₃; OH; OC₁₋₆alkyl; and C₃₋₆cycloalkyl; and
   n is 1, or 2;
or a pharmaceutically acceptable salt, solvate, stereoisomer, tautomer, isotopic variant, or N-oxide thereof.
23. The compound according to embodiment 1, having the structure of Formula (IC):

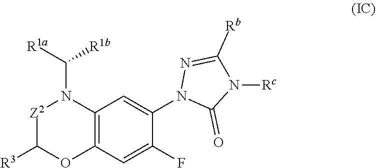

wherein
Z² is CH₂, or C═O;
R^{1a} is C₁₋₄alkyl;
R^{1b} is C₁₋₄alkyl or C₁₋₄haloalkyl;
R^{b} is C₁₋₄alkyl substituted with OH, halo, CN, OC₁₋₄alkyl, OC₁₋₄haloalkyl or OC₃₋₆cycloalkyl;
R^{c} is C₁₋₄alkyl, C₁₋₄haloalkyl, or C₃₋₆cycloalkyl;
R³ is

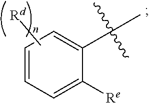

wherein
R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl; and
n is 1, or 2;
or a pharmaceutically acceptable salt, solvate, stereoisomer, tautomer, isotopic variant, or N-oxide thereof.
24. The compound according to embodiment 1, having the structure of Formula (ID):

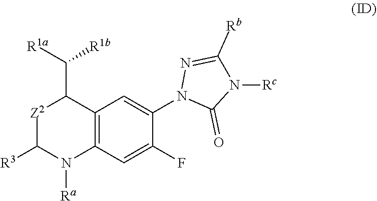

wherein
Z² is CH₂;
R^{1a} is C₁₋₄alkyl;
R^{1b} is C₁₋₄alkyl or C₁₋₄haloalkyl;
R^{a} is selected from the group consisting of: H, CH₂(C═O)NH₂, (C═O)CH₃, and (C═O)NHCH₃;
R^{b} is C₁₋₄alkyl substituted with OH, halo, CN, OC₁₋₄alkyl, OC₁₋₄haloalkyl or OC₃₋₆cycloalkyl;
R^{c} is C₁₋₄alkyl, C₁₋₄haloalkyl, or C₃₋₆cycloalkyl; and
R³ is

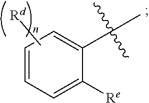

wherein
R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl; and
n is 1, or 2;
or a pharmaceutically acceptable salt, solvate, stereoisomer, tautomer, isotopic variant, or N-oxide thereof.
25. The compound according to embodiment 1, having the structure of Formula (IE):

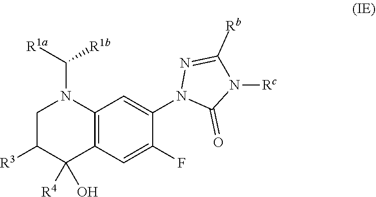

wherein
R^{1a} is C₁₋₄alkyl;
R^{1b} is C₁₋₄alkyl or C₁₋₄haloalkyl;
R^{b} is C₁₋₄alkyl substituted with OH, halo, CN, OC₁₋₄alkyl, OC₁₋₄haloalkyl or OC₃₋₆cycloalkyl;
R^{c} is C₁₋₄alkyl, C₁₋₄haloalkyl, or C₃₋₆cycloalkyl; and
R³ is

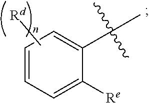

wherein
R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl;
n is 1, or 2; and
R⁴ is H or CH₃;
or a pharmaceutically acceptable salt, solvate, stereoisomer, tautomer, isotopic variant, or N-oxide thereof.
26. The compound according to embodiment 21, wherein R^{1b} is CF₃.
27. The compound according to embodiment 22, wherein R^{1a} is CH₃.
28. The compound according to embodiment 23, wherein R^{c} is C₁₋₄alkyl.
29. The compound according to embodiment 24, wherein R^{1a} and R^{1b} are CH₃.
30. The compound according to embodiment 25, wherein R^{1a} and R^{1b} are CH₃.
31. A compound selected from the group consisting of
7-(4-Ethyl-3-(hydroxymethyl)-5-oxo-4,5-dihydro-1H-1,2,4-triazol-1-yl)-6-fluoro-3-(3-fluorophenyl)-1-isopropylquinolin-4(1H)-one;
7-(4-Ethyl-3-(hydroxymethyl)-5-oxo-4,5-dihydro-1H-1,2,4-triazol-1-yl)-3-(2-(methylthio)phenyl)-1-((S)-1,1,1-trifluoropropan-2-yl)-3,4-dihydroquinoxalin-2(1H)-one;
3-(2-Chloro-6-fluorophenyl)-7-(4-ethyl-3-(hydroxymethyl)-5-oxo-4,5-dihydro-1H-1,2,4-triazol-1-yl)-1-((S)-1,1,1-trifluoropropan-2-yl)-3,4-dihydroquinoxalin-2(1H)-one;
2-(2-(2-Chloro-6-fluorophenyl)-4-((S)-1,1,1-trifluoropropan-2-yl)-1,2,3,4-tetrahydroquinoxalin-6-yl)-4-ethyl-5-(hydroxymethyl)-2,4-dihydro-3H-1,2,4-triazol-3-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-isopropyl-2H-benzo[b][1,4]oxazin-3(4H)-one;
1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one;
(S*)-1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one;
(R*)-1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one;
7-(4-Ethyl-3-(hydroxymethyl)-5-oxo-4,5-dihydro-TH-1,2,4-triazol-1-yl)-6-fluoro-3-(2-fluoro-5-methylphenyl)-1-isopropylquinolin-4(1H)-one;
7-(4-Ethyl-3-(hydroxymethyl)-5-oxo-4,5-dihydro-TH-1,2,4-triazol-1-yl)-6-fluoro-1-isopropyl-3-(o-tolyl)quinolin-4(1H)-one;
7-(4-Ethyl-3-(hydroxymethyl)-5-oxo-4,5-dihydro-1H-1,2,4-triazol-1-yl)-6-fluoro-1-isopropyl-3-(o-tolyl)-2,3-dihydroquinolin-4(1H)-one;
3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropylquinolin-4(1H)-one;
Racemic 4-Ethyl-2-(7-fluoro-4-isopropyl-2-(o-tolyl)-1,2,3,4-tetrahydroquinolin-6-yl)-5-(hydroxymethyl)-2,4-dihydro-3H-1,2,4-triazol-3-one;
Racemic 4-Ethyl-2-(6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3S*,4S*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3R*,4R*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3S*,4R*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3R*,4S*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
Racemic 7-(4-Ethyl-3-(hydroxymethyl)-5-oxo-4,5-dihydro-TH-1,2,4-triazol-1-yl)-6-fluoro-1-isopropyl-3-methyl-3-(o-tolyl)-2,3-dihydroquinolin-4(1H)-one;
Racemic 4-Ethyl-2-(6-fluoro-4-hydroxy-1-isopropyl-4-methyl-3-(o-tolyl)-1,2,3,4-tetrahydroquinolin-7-yl)-5-(hydroxymethyl)-2,4-dihydro-3H-1,2,4-triazol-3-one;
4-Ethyl-2-(6-fluoro-1-isopropyl-4-methyl-3-(o-tolyl)-1,2-dihydroquinolin-7-yl)-5-(hydroxymethyl)-2,4-dihydro-3H-1,2,4-triazol-3-one;
4-Ethyl-2-((2R*,4S*)-7-fluoro-4-isopropyl-2-(o-tolyl)-1,2,3,4-tetrahydroquinolin-6-yl)-5-(hydroxymethyl)-2,4-dihydro-3H-1,2,4-triazol-3-one;
4-Ethyl-2-((2R*,4S*)-7-fluoro-4-isopropyl-2-(o-tolyl)-1,2,3,4-tetrahydroquinolin-6-yl)-5-(hydroxymethyl)-2,4-dihydro-3H-1,2,4-triazol-3-one; and
1-(1-Acetyl-7-fluoro-4-isopropyl-2-(o-tolyl)-1,2,3,4-tetrahydroquinolin-6-yl)-4-ethyl-3-(hydroxymethyl)-1H-1,2,4-triazol-5(4H)-one;
   or a pharmaceutically acceptable salt, solvate, stereoisomer, tautomer, isotopic variant, or N-oxide thereof.
   32. A pharmaceutical composition comprising: (A) an effective amount of a compound according to any of embodiments 1-30, or a pharmaceutically acceptable salt, solvate, stereoisomer, tautomer, isotopic variant, or N-oxide thereof; and (B) at least one pharmaceutically acceptable excipient.
   33. A pharmaceutical composition comprising an effective amount of a compound of embodiment 31, or a pharmaceutically acceptable salt, solvate, stereoisomer, tautomer, isotopic variant, or N-oxide thereof; and at least one pharmaceutically acceptable excipient.
   34. A method of treating a subject suffering from or diagnosed with a disease, disorder, or medical condition comprising inhibiting or altering dihydroorotate oxygenase enzyme activity in the subject by administering to the subject an effective amount of at least one compound according to any of claims 1-30, or a pharmaceutically acceptable salt, solvate, stereoisomer, tautomer, isotopic variant, or N-oxide thereof.
   35. The method according to embodiment 34, wherein the disorder, disease or medical condition is selected from the group consisting of inflammatory disorders and autoimmune disorders.
   36. The method according to embodiment 34, wherein the disorder, disease or medical condition is cancer.
   37. The method according to embodiment 34, wherein the disorder, disease or medical condition is selected from the group consisting of: lymphomas, leukemias, carcinomas, and sarcomas.
   38. The method according to embodiment 34, wherein the disorder, disease or medical condition is selected from the group consisting of: acute lymphoblastic leukemia, acute myeloid leukemia, (acute) T-cell leukemia, acute lymphoblastic leukemia, acute lymphocytic leukemia, acute monocytic leukemia, acute promyelocytic leukemia, bisphenotypic B myelomonocytic leukemia, chronic lymphocytic leukemia, chronic myelogenous leukemia, chronic myeloid leukemia, chronic myelomonocytic leukemia, large granular lymphocytic leukemia, plasma cell leukemia, and also myelodysplastic syndrome, which can develop into an acute myeloid leukemia.
   39. The method according to embodiment 34, wherein the disorder, disease or medical condition is acute myeloid leukemia.
   40. The method according to any of embodiments 34-39, wherein the at least one compound comprises a compound selected from the group consisting of
7-(4-Ethyl-3-(hydroxymethyl)-5-oxo-4,5-dihydro-TH-1,2,4-triazol-1-yl)-6-fluoro-3-(3-fluorophenyl)-1-isopropylquinolin-4(1H)-one;
7-(4-Ethyl-3-(hydroxymethyl)-5-oxo-4,5-dihydro-1H-1,2,4-triazol-1-yl)-3-(2-(methylthio)phenyl)-1-((S)-1,1,1-trifluoropropan-2-yl)-3,4-dihydroquinoxalin-2(1H)-one;
3-(2-Chloro-6-fluorophenyl)-7-(4-ethyl-3-(hydroxymethyl)-5-oxo-4,5-dihydro-1H-1,2,4-triazol-1-yl)-1-((S)-1,1,1-trifluoropropan-2-yl)-3,4-dihydroquinoxalin-2(1H)-one;
2-(2-(2-Chloro-6-fluorophenyl)-4-((S)-1,1,1-trifluoropropan-2-yl)-1,2,3,4-tetrahydroquinoxalin-6-yl)-4-ethyl-5-(hydroxymethyl)-2,4-dihydro-3H-1,2,4-triazol-3-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-isopropyl-2H-benzo[b][1,4]oxazin-3(4H)-one;
1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one;
(S*)-1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one;
(R*)-1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one;
7-(4-Ethyl-3-(hydroxymethyl)-5-oxo-4,5-dihydro-TH-1,2,4-triazol-1-yl)-6-fluoro-3-(2-fluoro-5-methylphenyl)-1-isopropylquinolin-4(1H)-one;
7-(4-Ethyl-3-(hydroxymethyl)-5-oxo-4,5-dihydro-TH-1,2,4-triazol-1-yl)-6-fluoro-1-isopropyl-3-(o-tolyl)quinolin-4(1H)-one;
7-(4-Ethyl-3-(hydroxymethyl)-5-oxo-4,5-dihydro-TH-1,2,4-triazol-1-yl)-6-fluoro-1-isopropyl-3-(o-tolyl)-2,3-dihydroquinolin-4(1H)-one;
3-(2-Chloro-6-fluorophenyl)-7-(4-ethyl-3-(hydroxymethyl)-5-oxo-4,5-dihydro-TH-1,2,4-triazol-1-yl)-6-fluoro-1-isopropylquinolin-4(1H)-one;
Racemic 4-Ethyl-2-(7-fluoro-4-isopropyl-2-(o-tolyl)-1,2,3,4-tetrahydroquinolin-6-yl)-5-(hydroxymethyl)-2,4-dihydro-3H-1,2,4-triazol-3-one;
Racemic 4-Ethyl-2-(6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3S*,4S*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3R*,4R*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3S*,4R*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3R*,4S*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
Racemic 7-(4-Ethyl-3-(hydroxymethyl)-5-oxo-4,5-dihydro-1H-1,2,4-triazol-1-yl)-6-fluoro-1-isopropyl-3-methyl-3-(o-tolyl)-2,3-dihydroquinolin-4(1H)-one;
Racemic 4-Ethyl-2-(6-fluoro-4-hydroxy-1-isopropyl-4-methyl-3-(o-tolyl)-1,2,3,4-tetrahydroquinolin-7-yl)-5-(hydroxymethyl)-2,4-dihydro-3H-1,2,4-triazol-3-one;
4-Ethyl-2-(6-fluoro-1-isopropyl-4-methyl-3-(o-tolyl)-1,2-dihydroquinolin-7-yl)-5-(hydroxymethyl)-2,4-dihydro-3H-1,2,4-triazol-3-one;
4-Ethyl-2-((2R*,4S*)-7-fluoro-4-isopropyl-2-(0-tolyl)-1,2,3,4-tetrahydroquinolin-6-yl)-5-(hydroxymethyl)-2,4-dihydro-3H-1,2,4-triazol-3-one;
4-Ethyl-2-((2R*,4S*)-7-fluoro-4-isopropyl-2-(0-tolyl)-1,2,3,4-tetrahydroquinolin-6-yl)-5-(hydroxymethyl)-2,4-dihydro-3H-1,2,4-triazol-3-one; and
1-(1-Acetyl-7-fluoro-4-isopropyl-2-(o-tolyl)-1,2,3,4-tetrahydroquinolin-6-yl)-4-ethyl-3-(hydroxymethyl)-1H-1,2,4-triazol-5(4H)-one;
   or a pharmaceutically acceptable salt, solvate, stereoisomer, tautomer, isotopic variant, or N-oxide thereof.

1. A compound having the structure of Formula (I): 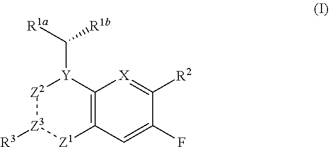
wherein
X is CH;
Y is CH or N;
Z¹ is selected from the group consisting of: CH₂, C(CH₃), CH(OH), C(CH₃)(OH), O, C═O, and NR^{a};
R^{a} is selected from the group consisting of: H, CH₂(C═O)NH₂, (C═O)CH₃, and (C═O)NHCH₃;
Z² is CH, CH₂, or C═O;
Z³ is C, CH or C(CH₃);
each [symbol] is independently a single bond or a double bond;
wherein
when Z³ is CH or C(CH₃), [symbol] between Z² and Z³ is a single bond, and [symbol] between Z³ and Z¹ is a single bond;
when Z³ is C, Z² is CH, [symbol] between Z² and Z³ is a double bond, and [symbol] between Z³ and Z¹ is a single bond;
or
when Z¹ is C(CH₃) [symbol] between Z² and Z³ is a single bond, and [symbol] between Z³ and Z¹ is a double bond;
R^{1a} is selected from the group consisting of: C₁₋₆alkyl; C₁₋₆alkyl substituted with OH, or OCH₃; C₁₋₆haloalkyl; C₁₋₆haloalkyl substituted with OH, or OCH₃; and C₃₋₆cycloalkyl;
R^{1b} is CH₃ or CHF₂; or R^{1a} and R^{1b} come together to form C₃₋₆cycloalkyl; C₃₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl;
R² is 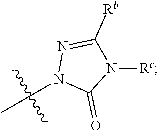
wherein
R^{b} is C₁₋₆alkyl substituted with a member selected from the group consisting of: OH, halo, CN, OC₁₋₆alkyl, OC₁₋₆haloalkyl and OC₃₋₆cycloalkyl; and
R^{c} is selected from the group consisting of: C₁₋₆alkyl, C₁₋₆haloalkyl, and C₃₋₆cycloalkyl;
R³ is 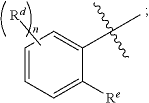
wherein
R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: H, halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl;
n is 1, or 2; and
R⁴ is H or CH₃;
or a pharmaceutically acceptable salt, solvate, stereoisomer, tautomer, isotopic variant, or N-oxide thereof.

2. The compound according to claim 1, wherein Y is CH; or a pharmaceutically acceptable salt, solvate, stereoisomer, tautomer, isotopic variant, or N-oxide thereof.

3. The compound according to claim 1, wherein Y is N; or a pharmaceutically acceptable salt, solvate, stereoisomer, tautomer, isotopic variant, or N-oxide thereof.

4. The compound according to claim 1, wherein 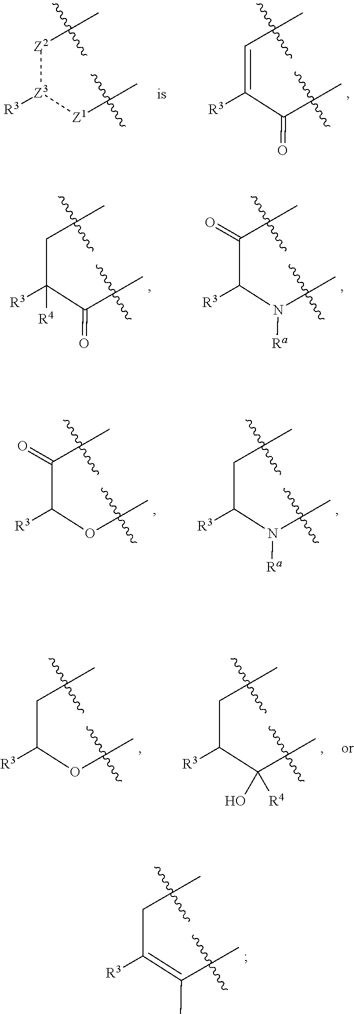
and R⁴ is H or CH₃; or a pharmaceutically acceptable salt, solvate, stereoisomer, tautomer, isotopic variant, or N-oxide thereof.

5. The compound according to claim 1, wherein 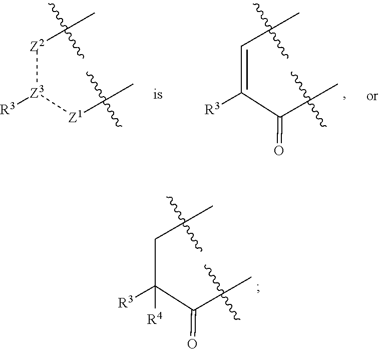
or a pharmaceutically acceptable salt, solvate, stereoisomer, tautomer, isotopic variant, or N-oxide thereof.

6. The compound according to claim 1, wherein 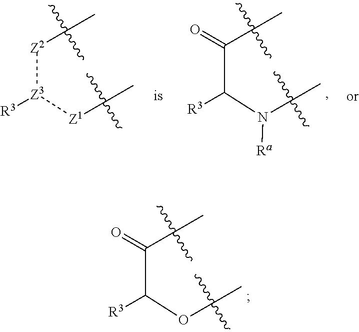
or a pharmaceutically acceptable salt, solvate, stereoisomer, tautomer, isotopic variant, or N-oxide thereof.

7. The compound according to claim 1, wherein 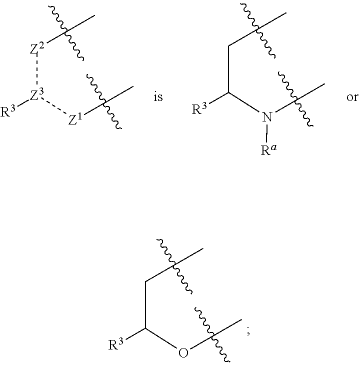
or a pharmaceutically acceptable salt, solvate, stereoisomer, tautomer, isotopic variant, or N-oxide thereof.

8. The compound according to claim 1, wherein 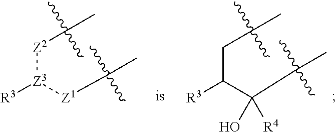
or a pharmaceutically acceptable salt, solvate, stereoisomer, tautomer, isotopic variant, or N-oxide thereof.

9. The compound according to claim 1, wherein 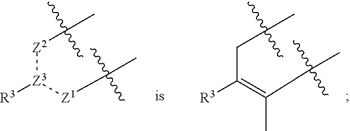
or a pharmaceutically acceptable salt, solvate, stereoisomer, tautomer, isotopic variant, or N-oxide thereof.

10. The compound according to claim 1, wherein R^{a} is C₁₋₄alkyl substituted with OH; CH₂(C═O)NH₂, (C═O)CH₃, and (C═O)NHCH₃; or a pharmaceutically acceptable salt, solvate, stereoisomer, tautomer, isotopic variant, or N-oxide thereof.

11. The compound according to claim 1, wherein R^{1a} is C₁₋₄alkyl; C₁₋₄alkyl substituted with OH, or OCH₃; C₁₋₄haloalkyl; C₁₋₄haloalkyl substituted with OH, or OCH₃; or C₃₋₆cycloalkyl; or a pharmaceutically acceptable salt, solvate, stereoisomer, tautomer, isotopic variant, or N-oxide thereof.

12. The compound according to claim 1, wherein R^{1a} is CH₃ or CF₃; or a pharmaceutically acceptable salt, solvate, stereoisomer, tautomer, isotopic variant, or N-oxide thereof.

13. The compound according to claim 1, wherein R^{1b} is CH₃ or CHF₂; or a pharmaceutically acceptable salt, solvate, stereoisomer, tautomer, isotopic variant, or N-oxide thereof.

14. The compound according to claim 1, wherein R^{1b} is CH₃; or a pharmaceutically acceptable salt, solvate, stereoisomer, tautomer, isotopic variant, or N-oxide thereof.

15. The compound according to claim 1, wherein R^{1a} and R^{1b} come together to form cyclopropyl, cyclobutyl, cyclopentyl, or cyclohexyl; cyclopropyl, cyclobutyl, cyclopentyl, or cyclohexyl each independently substituted with one, two, three or four members selected from the group consisting of: halo, OH, C₁₋₄alkyl, and C₁₋₄haloalkyl; oxetanyl; tetrahydrofuranyl; and tetrahydropyranyl; or a pharmaceutically acceptable salt, solvate, stereoisomer, tautomer, isotopic variant, or N-oxide thereof.

16. The compound according to claim 1, wherein R² is 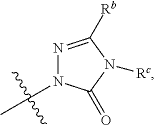
where
R^{b} is C₁₋₄alkyl substituted with OH, halo, CN, OC₁₋₄alkyl, OC₁₋₄haloalkyl or OC₃₋₆cycloalkyl; and
R^{c} is C₁₋₄alkyl, C₁₋₄haloalkyl, or C₃₋₆cycloalkyl; or a pharmaceutically acceptable salt, solvate, stereoisomer, tautomer, isotopic variant, or N-oxide thereof.

17. The compound according to claim 1, wherein R² is 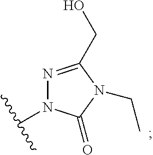 [SMILES: *n1nc(CO)n(CC)c1=O]
or a pharmaceutically acceptable salt, solvate, stereoisomer, tautomer, isotopic variant, or N-oxide thereof.

18. The compound according to claim 1, wherein R³ is 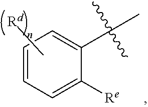
where
R^{d} is H; halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; CN; or OC₁₋₄alkyl;
R^{e} is H, halo; C₁₋₄alkyl; C₁₋₄alkyl substituted with OH, OCH₃, SCH₃, or OCF₃;
C₁₋₄haloalkyl; or C₁₋₄haloalkyl substituted with OH, or OCH₃; and
n is 1 or 2;
or a pharmaceutically acceptable salt, solvate, stereoisomer, tautomer, isotopic variant, or N-oxide thereof.

19. The compound according to claim 18, wherein R^{e} is H, SCH₃, Cl, F, or CH₃; or a pharmaceutically acceptable salt, solvate, stereoisomer, tautomer, isotopic variant, or N-oxide thereof.

20. The compound according to claim 1, having the structure of Formula (IA): 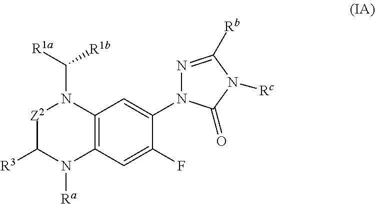
wherein
Z² is CH₂ or C═O;
R^{1a} is C₁₋₄alkyl;
R^{1b} is C₁₋₄alkyl or C₁₋₄haloalkyl;
R^{a} is H, C₁₋₆alkyl substituted with OH; CH₂(C═O)NH₂, (C═O)CH₃, and (C═O)NHCH₃;
R^{b} is C₁₋₄alkyl substituted with a member selected from the group consisting of: OH, halo, CN, OC₁₋₄alkyl, OC₁₋₄haloalkyl and OC₃₋₆cycloalkyl;
R^{c} is selected from the group consisting of: C₁₋₄alkyl, C₁₋₄haloalkyl, and C₃₋₆cycloalkyl; and
R³ is 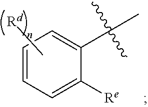
R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl; and
n is 1, or 2;
or a pharmaceutically acceptable salt, solvate, stereoisomer, tautomer, isotopic variant, or N-oxide thereof.

21. The compound according to claim 1, having the structure of Formula (IB): 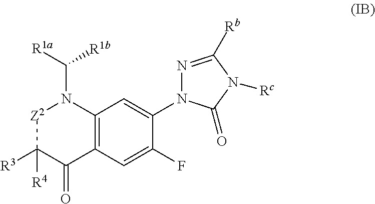
wherein
when Z² is CH, [symbol] is a double bond and R⁴ is absent; when Z² is CH₂, [symbol] is a single bond and R⁴ is H or CH₃;
R^{1a} is C₁₋₄alkyl;
R^{1b} is C₁₋₄alkyl or C₁₋₄haloalkyl;
R^{b} is C₁₋₄alkyl substituted with OH, halo, CN, OC₁₋₄alkyl, OC₁₋₄haloalkyl or OC₃₋₆cycloalkyl;
R^{c} is C₁₋₄alkyl, C₁₋₄haloalkyl, or C₃₋₆cycloalkyl; and
R³ is 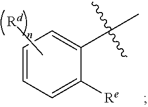
wherein
R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl; and
n is 1, or 2;
or a pharmaceutically acceptable salt, solvate, stereoisomer, tautomer, isotopic variant, or N-oxide thereof.

22. The compound according to claim 1, having the structure of Formula (IC): 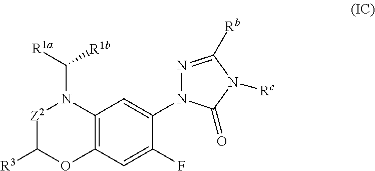
wherein
Z² is CH₂, or C═O;
R^{1a} is C₁₋₄alkyl;
R^{1b} is C₁₋₄alkyl or C₁₋₄haloalkyl;
R^{b} is C₁₋₄alkyl substituted with OH, halo, CN, OC₁₋₄alkyl, OC₁₋₄haloalkyl or OC₃₋₆cycloalkyl;
R^{c} is C₁₋₄alkyl, C₁₋₄haloalkyl, or C₃₋₆cycloalkyl;
R³ is 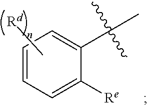
wherein
R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl; and
n is 1, or 2;
or a pharmaceutically acceptable salt, solvate, stereoisomer, tautomer, isotopic variant, or N-oxide thereof.

23. The compound according to claim 1, having the structure of Formula (ID): 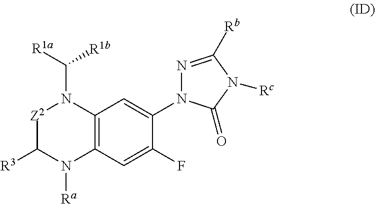
wherein
Z² is CH₂;
R^{1a} is C₁₋₄alkyl;
R^{1b} is C₁₋₄alkyl or C₁₋₄haloalkyl;
R^{a} is selected from the group consisting of H, CH₂(C═O)NH₂, (C═O)CH₃, and (C═O)NHCH₃;
R^{b} is C₁₋₄alkyl substituted with OH, halo, CN, OC₁₋₄alkyl, OC₁₋₄haloalkyl or OC₃₋₆cycloalkyl;
R^{c} is C₁₋₄alkyl, C₁₋₄haloalkyl, or C₃₋₆cycloalkyl; and
R³ is 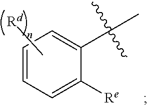
wherein
R^{d} is selected from the group consisting of H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl; and
n is 1, or 2;
or a pharmaceutically acceptable salt, solvate, stereoisomer, tautomer, isotopic variant, or N-oxide thereof.

24. The compound according to claim 1, having the structure of Formula (IE): 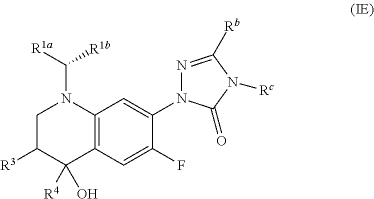
wherein
R^{1a} is C₁₋₄alkyl;
R^{1b} is C₁₋₄alkyl or C₁₋₄haloalkyl;
R^{b} is C₁₋₄alkyl substituted with OH, halo, CN, OC₁₋₄alkyl, OC₁₋₄haloalkyl or OC₃₋₆cycloalkyl;
R^{c} is C₁₋₄alkyl, C₁₋₄haloalkyl, or C₃₋₆cycloalkyl; and
R³ is 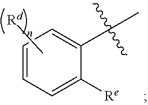
wherein
R^{d} is selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl;
n is 1, or 2; and
R⁴ is H or CH₃;
or a pharmaceutically acceptable salt, solvate, stereoisomer, tautomer, isotopic variant, or N-oxide thereof.

25. The compound according to claim 20, wherein R^{1b} is CF₃.

26. The compound according to claim 21, wherein R^{1a} is CH₃.

27. The compound according to claim 22, wherein R^{c} is C₁₋₄alkyl.

28. The compound according to claim 23, wherein R^{1a} and R^{1b} are CH₃.

29. The compound according to claim 24, wherein R^{1a} and R^{1b} are CH₃.

30. A compound selected from the group consisting of
7-(4-Ethyl-3-(hydroxymethyl)-5-oxo-4,5-dihydro-1H-1,2,4-triazol-1-yl)-6-fluoro-3-(3-fluorophenyl)-1-isopropylquinolin-4(1H)-one;
7-(4-Ethyl-3-(hydroxymethyl)-5-oxo-4,5-dihydro-1H-1,2,4-triazol-1-yl)-3-(2-(methylthio)phenyl)-1-((S)-1,1,1-trifluoropropan-2-yl)-3,4-dihydroquinoxalin-2(1H)-one;
3-(2-Chloro-6-fluorophenyl)-7-(4-ethyl-3-(hydroxymethyl)-5-oxo-4,5-dihydro-1H-1,2,4-triazol-1-yl)-1-((S)-1,1,1-trifluoropropan-2-yl)-3,4-dihydroquinoxalin-2(1H)-one;
2-(2-(2-Chloro-6-fluorophenyl)-4-((S)-1,1,1-trifluoropropan-2-yl)-1,2,3,4-tetrahydroquinoxalin-6-yl)-4-ethyl-5-(hydroxymethyl)-2,4-dihydro-3H-1,2,4-triazol-3-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-isopropyl-2H-benzo[b][1,4]oxazin-3(4H)-one;
1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one;
(S*)-1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one;
(R*)-1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one;
7-(4-Ethyl-3-(hydroxymethyl)-5-oxo-4,5-dihydro-1H-1,2,4-triazol-1-yl)-6-fluoro-3-(2-fluoro-5-methylphenyl)-1-isopropylquinolin-4(1H)-one;
7-(4-Ethyl-3-(hydroxymethyl)-5-oxo-4,5-dihydro-1H-1,2,4-triazol-1-yl)-6-fluoro-1-isopropyl-3-(o-tolyl)quinolin-4(1H)-one;
7-(4-Ethyl-3-(hydroxymethyl)-5-oxo-4,5-dihydro-TH-1,2,4-triazol-1-yl)-6-fluoro-1-isopropyl-3-(o-tolyl)-2,3-dihydroquinolin-4(1H)-one;
3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropylquinolin-4(1H)-one;
Racemic 4-Ethyl-2-(7-fluoro-4-isopropyl-2-(o-tolyl)-1,2,3,4-tetrahydroquinolin-6-yl)-5-(hydroxymethyl)-2,4-dihydro-3H-1,2,4-triazol-3-one;
Racemic 4-Ethyl-2-(6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3S*,4S*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3R*,4R*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3S*,4R*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3R*,4S*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
Racemic 7-(4-Ethyl-3-(hydroxymethyl)-5-oxo-4,5-dihydro-TH-1,2,4-triazol-1-yl)-6-fluoro-1-isopropyl-3-methyl-3-(o-tolyl)-2,3-dihydroquinolin-4(1H)-one;
Racemic 4-Ethyl-2-(6-fluoro-4-hydroxy-1-isopropyl-4-methyl-3-(o-tolyl)-1,2,3,4-tetrahydroquinolin-7-yl)-5-(hydroxymethyl)-2,4-dihydro-3H-1,2,4-triazol-3-one;
4-Ethyl-2-(6-fluoro-1-isopropyl-4-methyl-3-(o-tolyl)-1,2-dihydroquinolin-7-yl)-5-(hydroxymethyl)-2,4-dihydro-3H-1,2,4-triazol-3-one;
4-Ethyl-2-((2R*,4S*)-7-fluoro-4-isopropyl-2-(0-tolyl)-1,2,3,4-tetrahydroquinolin-6-yl)-5-(hydroxymethyl)-2,4-dihydro-3H-1,2,4-triazol-3-one;
4-Ethyl-2-((2R*,4S*)-7-fluoro-4-isopropyl-2-(0-tolyl)-1,2,3,4-tetrahydroquinolin-6-yl)-5-(hydroxymethyl)-2,4-dihydro-3H-1,2,4-triazol-3-one; and
1-(1-Acetyl-7-fluoro-4-isopropyl-2-(o-tolyl)-1,2,3,4-tetrahydroquinolin-6-yl)-4-ethyl-3-(hydroxymethyl)-1H-1,2,4-triazol-5(4H)-one;
or a pharmaceutically acceptable salt, solvate, stereoisomer, tautomer, isotopic variant, or N-oxide thereof.

31. A pharmaceutical composition comprising: (A) an effective amount of a compound according to claim 1, or a pharmaceutically acceptable salt, solvate, stereoisomer, tautomer, isotopic variant, or N-oxide thereof; and (B) at least one pharmaceutically acceptable excipient.

32. A pharmaceutical composition comprising an effective amount of a compound of claim 30, or a pharmaceutically acceptable salt, solvate, stereoisomer, tautomer, isotopic variant, or N-oxide thereof, and at least one pharmaceutically acceptable excipient.

33. A method of treating a subject suffering from or diagnosed with a disease, disorder, or medical condition comprising inhibiting or altering dihydroorotate oxygenase enzyme activity in the subject by administering to the subject an effective amount of at least one compound according to claim 1, or a pharmaceutically acceptable salt, solvate, stereoisomer, tautomer, isotopic variant, or N-oxide thereof.

34. The method according to claim 33, wherein the disorder, disease or medical condition is selected from the group consisting of: inflammatory disorders and autoimmune disorders.

35. The method according to claim 33, wherein the disorder, disease or medical condition is cancer.

36. The method according to claim 33, wherein the disorder, disease or medical condition is selected from the group consisting of: lymphomas, leukemias, carcinomas, and sarcomas.

37. The method according to claim 33, wherein the disorder, disease or medical condition is selected from the group consisting of: acute lymphoblastic leukemia, acute myeloid leukemia, (acute) T-cell leukemia, acute lymphoblastic leukemia, acute lymphocytic leukemia, acute monocytic leukemia, acute promyelocytic leukemia, bisphenotypic B myelomonocytic leukemia, chronic lymphocytic leukemia, chronic myelogenous leukemia, chronic myeloid leukemia, chronic myelomonocytic leukemia, large granular lymphocytic leukemia, plasma cell leukemia, and also myelodysplastic syndrome, which can develop into an acute myeloid leukemia.

38. The method according to claim 33, wherein the disorder, disease or medical condition is acute myeloid leukemia.

39. The method according to claim 33, wherein the at least one compound comprises a compound selected from the group consisting of:
7-(4-Ethyl-3-(hydroxymethyl)-5-oxo-4,5-dihydro-1H-1,2,4-triazol-1-yl)-6-fluoro-3-(3-fluorophenyl)-1-isopropylquinolin-4(1H)-one;
7-(4-Ethyl-3-(hydroxymethyl)-5-oxo-4,5-dihydro-1H-1,2,4-triazol-1-yl)-3-(2-(methylthio)phenyl)-1-((S)-1,1,1-trifluoropropan-2-yl)-3,4-dihydroquinoxalin-2(1H)-one;
3-(2-Chloro-6-fluorophenyl)-7-(4-ethyl-3-(hydroxymethyl)-5-oxo-4,5-dihydro-1H-1,2,4-triazol-1-yl)-1-((S)-1,1,1-trifluoropropan-2-yl)-3,4-dihydroquinoxalin-2(1H)-one;
2-(2-(2-Chloro-6-fluorophenyl)-4-((S)-1,1,1-trifluoropropan-2-yl)-1,2,3,4-tetrahydroquinoxalin-6-yl)-4-ethyl-5-(hydroxymethyl)-2,4-dihydro-3H-1,2,4-triazol-3-one;
2-(2-Chloro-6-fluorophenyl)-6-(4-ethyl-3-(hydroxymethyl)-5-oxo-4,5-dihydro-1H-1,2,4-triazol-1-yl)-7-fluoro-4-isopropyl-2H-benzo[b][1,4]oxazin-3(4H)-one;
1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one;
(S*)-1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one;
(R*)-1-(2-(2-Chloro-6-fluorophenyl)-7-fluoro-4-isopropyl-3,4-dihydro-2H-benzo[b][1,4]oxazin-6-yl)-4-ethyl-3-(hydroxymethyl)-1H-1,2,4-triazol-5(4H)-one;
7-(4-Ethyl-3-(hydroxymethyl)-5-oxo-4,5-dihydro-TH-1,2,4-triazol-1-yl)-6-fluoro-3-(2-fluoro-5-methylphenyl)-1-isopropylquinolin-4(1H)-one;
7-(4-Ethyl-3-(hydroxymethyl)-5-oxo-4,5-dihydro-1H-1,2,4-triazol-1-yl)-6-fluoro-1-isopropyl-3-(o-tolyl)quinolin-4(1H)-one;
7-(4-Ethyl-3-(hydroxymethyl)-5-oxo-4,5-dihydro-1H-1,2,4-triazol-1-yl)-6-fluoro-1-isopropyl-3-(o-tolyl)-2,3-dihydroquinolin-4(1H)-one;
3-(2-Chloro-6-fluorophenyl)-7-(4-ethyl-3-(hydroxymethyl)-5-oxo-4,5-dihydro-1H-1,2,4-triazol-1-yl)-6-fluoro-1-isopropylquinolin-4(1H)-one;
Racemic 4-Ethyl-2-(7-fluoro-4-isopropyl-2-(o-tolyl)-1,2,3,4-tetrahydroquinolin-6-yl)-5-(hydroxymethyl)-2,4-dihydro-3H-1,2,4-triazol-3-one;
Racemic 4-Ethyl-2-(6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3S*,4S*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3R*,4R*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3S*,4R*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
4-Ethyl-2-((3R*,4S*)-6-fluoro-4-hydroxy-1-isopropyl-3-(o-tolyl)-1,2,3,4-tetrahydroquinolin-7-yl)-5-(hydroxymethyl)-2,4-dihydro-3H-1,2,4-triazol-3-one;
Racemic 7-(4-Ethyl-3-(hydroxymethyl)-5-oxo-4,5-dihydro-TH-1,2,4-triazol-1-yl)-6-fluoro-1-isopropyl-3-methyl-3-(o-tolyl)-2,3-dihydroquinolin-4(1H)-one;
Racemic 4-Ethyl-2-(6-fluoro-4-hydroxy-1-isopropyl-4-methyl-3-(o-tolyl)-1,2,3,4-tetrahydroquinolin-7-yl)-5-(hydroxymethyl)-2,4-dihydro-3H-1,2,4-triazol-3-one;
4-Ethyl-2-(6-fluoro-1-isopropyl-4-methyl-3-(o-tolyl)-1,2-dihydroquinolin-7-yl)-5-(hydroxymethyl)-2,4-dihydro-3H-1,2,4-triazol-3-one;
4-Ethyl-2-((2R*,4S*)-7-fluoro-4-isopropyl-2-(o-tolyl)-1,2,3,4-tetrahydroquinolin-6-yl)-5-(hydroxymethyl)-2,4-dihydro-3H-1,2,4-triazol-3-one;
4-Ethyl-2-((2R*,4S*)-7-fluoro-4-isopropyl-2-(o-tolyl)-1,2,3,4-tetrahydroquinolin-6-yl)-5-(hydroxymethyl)-2,4-dihydro-3H-1,2,4-triazol-3-one; and
1-(1-Acetyl-7-fluoro-4-isopropyl-2-(o-tolyl)-1,2,3,4-tetrahydroquinolin-6-yl)-4-ethyl-3-(hydroxymethyl)-1H-1,2,4-triazol-5(4H)-one;
or a pharmaceutically acceptable salt, solvate, stereoisomer, tautomer, isotopic variant, or N-oxide thereof.
